SEQUENCE LISTING (48 sequences)

SEQ ID NO: 1 - protein, 873 aa, Human
MGSLLALLALLPLWGAVAEGPAKKVLTLEGDLVLGGLFPVHQKGGPAEDCGPVNEHRGIQRLEAMLFALDRINRDPHLLPGVRLGAHILDSCSKDTHALEQALDFVRASLSRGADGSRHICPDGSYATHGDAPTAITGVIGGSYSDVSIQVANLLRLFQIPQISYASTSAKLSDKSRYDYFARTVPPDFFQAKAMAEILRFFNWTYVSTVASEGDYGETGIEAFELEARARNICVATSEKVGRAMSRAAFEGVVRALLQKPSARVAVLFTRSEDARELLAASQRLNASFTWVASDGWGALESVVAGSEGAAEGAITIELASYPISDFASYFQSLDPWNNSRNPWFREFWEQRFRCSFRQRDCAAHSLRAVPFEQESKIMFVVNAVYAMAHALHNMHRALCPNTTRLCDAMRPVNGRRLYKDFVLNVKFDAPFRPADTHNEVRFDRFGDGIGRYNIFTYLRAGSGRYRYQKVGYWAEGLTLDTSLIPWASPSAGPLPASRCSEPCLQNEVKSVQPGEVCCWLCIPCQPYEYRLDEFTCADCGLGYWPNASLTGCFELPQEYIRWGDAWAVGPVTIACLGALATLFVLGVFVRHNATPVVKASGRELCYILLGGVFLCYCMTFIFIAKPSTAVCTLRRLGLGTAFSVCYSALLTKTNRIARIFGGAREGAQRPRFISPASQVAICLALISGQLLIVVAWLVVEAPGTGKETAPERREVVTLRCNHRDASMLGSLAYDVLLIALCTLYAFKTRKCPENFNEAKFIGFTMYTTCIIWLAFLPIFYVTSSDYRVQTTTMCVSVSLSGSVVLGCLFAPKLHIILFQPQKNVVSHRAPTSRFGSAAARASSSLGQGSGSQFVPTVCNGREVVDSTTSSLT

SEQ ID NO: 2 - protein, 873 aa, Human
MGSLLALLALLPLWGAVAEGPAKKVLTLEGDLVLGGLFPVHQKGGPAEDCGPVNEHRGIQRLEAMLFALDRINRDPHLLPGVRLGAHILDSCSKDTHALEQALDFVRASLSRGADGSRHICPDGSYATHGDAPTAITGVIGGSYSDVSIQVANLLRLFQIPQISYASTSAKLSDKSRYDYFARTVPPDFFQAKAMAEILRFFNWTYVSTVASEGDYGETGIEAFELEARARNICVATSEKVGRAMSRAAFEGVVRALLQKPSARVAVLFTRSEDARELLAASQRLNASFTWVASDGWGALESVVAGSEGAAEGAITIELASYPISDFASYFQSLDPWNNSRNPWFREFWEQRFRCSFRQRDCAAHSLRAVPFEQESKIMFVVNAVYAMAHALHNMHRALCPNTTRLCDAMRPVNGRRLYKDFVLNVKFDAPFRPADTHNEVRFDRFGDGIGRYNIFTYLRAGSGRYRYQKVGYWAEGLTLDTSLIPWASPSAGPLPASRCSEPCLQNEVKSVQPGEVCCWLCIPCQPYEYRLDEFTCADCGLGYWPNASLTGCFELPQEYIRWGDAWAVGPVTIACLGALATLFVLGVFVRHNATPVVKASGRELCYILLGGVFLCYCMTFIFIAKPSTAVCTLRRLGLGTAFSVCYSALLTKTNRIARIFGGAREGAQRPRFISPASQVAICLALISVQLLIVVAWLVVEAPGTGKETAPERREVVTLRCNHRDASMLGSLAYDVLLIALCTLYAFKTRKCPENFNEAKFIGFTMYTTCIIWLAFLPIFYVTSSDYRVQTTTMCVSVSLSGSVVLGCLFAPKLHIILFQPQKNVVSHRAPTSRFGSAAARASSSLGQGSGSQFVPTVCNGREVVDSTTSSLT

SEQ ID NO: 3 - protein, 873 aa, Human
MGSLLALLALLPLWGAVAEGPAKKVLTLEGDLVLGGLFPVHQKGGPAEDCGPVNEHRGIQRLEAMLFALDRINRDPHLLPGVRLGAHILDSCSKDTHALEQALDFVRASLSRGADGSRHICPDGSYATHGDAPTAITGVIGGSYSDVSIQVANLLRLFQIPQISYASTSAKLSDKSRYDYFARTVPPDFFQAKAMAEILRFFNWTYVSTVASEGDYGETGIEAFELEARARNICVATSEKVGRAMSRAAFEGVVRALLQKPSARVAVLFTRSEDARELLAASQRLNASFTWVASDGWGALESVVAGSEGAAEGAITIELASYPISDFASYFQSLDPWNNSRNPWFREFWEQRFRCSFRQRDCAAHSLRAVPFEQESKIMFVVNAVYAMAHALHNMHRALCPNTTRLCDAMRPVNGRRLYKDFVLNVKFDAPFRPADTHNEVRFDRFGDGIGRYNIFTYLRAGSGRYRYQKVGYWAEGLTLDTSLIPWASPSAGPLPASRCSEPCLQNEVKSVQPGEVCCWLCIPCQPYEYRLDEFTCADCGLGYWPNASLTGCFELPQEYIRWGDAWAVGPVTIACLGALATLFVLGVFVRHNATPVVKASGRELCYILLGGVFLCYCMTFIFIAKPSTAVCTLRRLGLGTAFSVCYSALLTKTNRIARIFGGAREGAQRPRFISPASQVAICLALILVQLLIVVAWLVVEAPGTGKETAPERREVVTLRCNHRDASMLGSLAYDVLLIALCTLYAFKTRKCPENFNEAKFIGFTMYTTCIIWLAFLPIFYVTSSDYRVQTTTMCVSVSLSGSVVLGCLFAPKLHIILFQPQKNVVSHRAPTSRFGSAAARASSSLGQGSGSQFVPTVCNGREVVDSTTSSLT

SEQ ID NO: 4 - protein, 873 aa, Human
MGSLLALLALLPLWGAVAEGPAKKVLTLEGDLVLGGLFPVHQKGGPAEDCGPVNEHRGIQRLEAMLFALDRINRDPHLLPGVRLGAHILDSCSKDTHALEQALDFVRASLSRGADGSRHICPDGSYATHGDAPTAITGVIGGSYSDVSIQVANLLRLFQIPQISYASTSAKLSDKSRYDYFARTVPPDFFQAKAMAEILRFFNWTYVSTVASEGDYGETGIEAFELEARARNICVATSEKVGRAMSRAAFEGVVRALLQKPSARVAVLFTRSEDARELLAASQRLNASFTWVASDGWGALESVVAGSEGAAEGAITIELASYPISDFASYFQSLDPWNNSRNPWFREFWEQRFRCSFRQRDCAAHSLRAVPFEQESKIMFVVNAVYAMAHALHNMHRALCPNTTRLCDAMRPVNGRRLYKDFVLNVKFDAPFRPADTHNEVRFDRFGDGIGRYNIFTYLRAGSGRYRYQKVGYWAEGLTLDTSLIPWASPSAGPLPASRCSEPCLQNEVKSVQPGEVCCWLCIPCQPYEYRLDEFTCADCGLGYWPNASLTGCFELPQEYIRWGDAWAVGPVTIACLGALATLFVLGVFVRHNATPVVKASGRELCYILLGGVFLCYCMTFIFIAKPSTAVCTLRRLGLGTAFSVCYSALLTKTNRIARIFGGAREGAQRPRFISPASQVAICLALILGQLLIVVAWLVVEAPGTGKETAPERREVVTLRCNHRDASMLGSLAYNVLLIALCTLYAFKTRKCPENFNEAKFIGFTMYTTCIIWLAFLPIFYVTSSDYRVQTTTMCVSVSLSGSVVLGCLFAPKLHIILFQPQKNVVSHRAPTSRFGSAAARASSSLGQGSGSQFVPTVCNGREVVDSTTSSLT

SEQ ID NO: 5 - protein, 873 aa, Human
MGSLLALLALLPLWGAVAEGPAKKVLTLEGDLVLGGLFPVHQKGGPAEDCGPVNEHRGIQRLEAMLFALDRINRDPHLLPGVRLGAHILDSCSKDTHALEQALDFVRASLSRGADGSRHICPDGSYATHGDAPTAITGVIGGSYSDVSIQVANLLRLFQIPQISYASTSAKLSDKSRYDYFARTVPPDFFQAKAMAEILRFFNWTYVSTVASEGDYGETGIEAFELEARARNICVATSEKVGRAMSRAAFEGVVRALLQKPSARVAVLFTRSEDARELLAASQRLNASFTWVASDGWGALESVVAGSEGAAEGAITIELASYPISDFASYFQSLDPWNNSRNPWFREFWEQRFRCSFRQRDCAAHSLRAVPFEQESKIMFVVNAVYAMAHALHNMHRALCPNTTRLCDAMRPVNGRRLYKDFVLNVKFDAPFRPADTHNEVRFDRFGDGIGRYNIFTYLRAGSGRYRYQKVGYWAEGLTLDTSLIPWASPSAGPLPASRCSEPCLQNEVKSVQPGEVCCWLCIPCQPYEYRLDEFTCADCGLGYWPNASLTGCFELPQEYIRWGDAWAVGPVTIACLGALATLFVLGVFVRHNATPVVKASGRELCYILLGGVFLCYCMTFIFIAKPSTAVCTLRRLGLGTAFSVCYSALLTKTNRIARIFGGAREGAQRPRFISPASQVAICLALILVQLLIVVAWLVVEAPGTGKETAPERREVVTLRCNHRDASMLGSLAYNVLLIALCTLYAFKTRKCPENFNEAKFIGFTMYTTCIIWLAFLPIFYVTSSDYRVQTTTMCVSVSLSGSVVLGCLFAPKLHIILFQPQKNVVSHRAPTSRFGSAAARASSSLGQGSGSQFVPTVCNGREVVDSTTSSLT

SEQ ID NO: 6 - protein, 873 aa, Human
MGSLLALLALLPLWGAVAEGPAKKVLTLEGDLVLGGLFPVHQKGGPAEDCGPVNEHRGIQRLEAMLFALDRINRDPHLLPGVRLGAHILDSCSKDTHALEQALDFVRASLSRGADGSRHICPDGSYATHGDAPTAITGVIGGSYSDVSIQVANLLRLFQIPQISYASTSAKLSDKSRYDYFARTVPPDFFQAKAMAEILRFFNWTYVSTVASEGDYGETGIEAFELEARARNICVATSEKVGRAMSRAAFEGVVRALLQKPSARVAVLFTRSEDARELLAASQRLNASFTWVASDGWGALESVVAGSEGAAEGAITIELASYPISDFASYFQSLDPWNNSRNPWFREFWEQRFRCSFRQRDCAAHSLRAVPFEQESKIMFVVNAVYAMAHALHNMHRALCPNTTRLCDAMRPVNGRRLYKDFVLNVKFDAPFRPADTHNEVRFDRFGDGIGRYNIFTYLRAGSGRYRYQKVGYWAEGLTLDTSLIPWASPSAGPLPASRCSEPCLQNEVKSVQPGEVCCWLCIPCQPYEYRLDEFTCADCGLGYWPNASLTGCFELPQEYIRWGDAWAVGPVTIACLGALATLFVLGVFVRHNATPVVKASGRELCYILLGGVFLCYCMTFIFIAKPSTAVCTLRRLGLGTAFSVCYSALLTKTNRIARIFGGAREGAQRPRFISPASQVAICLALILGQLLIVVAWLVVEAPGTGKETAPERREVVTLRCNHRDASMLGSLAYDVLLIALCTLYAFKTRKCPENFNEAKFIGFTMYTTCIIWLAFLPIFYVTSSDYRVQTTTMCVSVSLSGSVVLGCLFAPKLHIILFQPQKNVVSHRAPTSRFGSAAARASSSLGQGSGSQFVPTVCNGREVVDSTTSSLT

SEQ ID NO: 7 - protein, 873 aa, Human
MGSLLALLALLPLWGAVAEGPAKKVLTLEGDLVLGGLFPVHQKGGPAEDCGPVNEHRGIQRLEAMLFALDRINRDPHLLPGVRLGAHILDSCSKDTHALEQALDFVRASLSRGADGSRHICPDGSYATHGDAPTAITGVIGGSYSDVSIQVANLLRLFQIPQISYASTSAKLSDKSRYDYFARTVPPDFFQAKAMAEILRFFNWTYVSTVASEGDYGETGIEAFELEARARNICVATSEKVGRAMSRAAFEGVVRALLQKPSARVAVLFTRSEDARELLAASQRLNASFTWVASDGWGALESVVAGSEGAAEGAITIELASYPISDFASYFQSLDPWNNSRNPWFREFWEQRFRCSFRQRDCAAHSLRAVPFEQESKIMFVVNAVYAMAHALHNMHRALCPNTTRLCDAMRPVNGRRLYKDFVLNVKFDAPFRPADTHNEVRFDRFGDGIGRYNIFTYLRAGSGRYRYQKVGYWAEGLTLDTSLIPWASPSAGPLPASRCSEPCLQNEVKSVQPGEVCCWLCIPCQPYEYRLDEFTCADCGLGYWPNASLTGCFELPQEYIRWGDAWAVGPVTIACLGALATLFVLGVFVRHNATPVVKASGRELCYILLGGVFLCYCMTFIFIAKPSTAVCTLRRLGLGTAFSVCYSALLTKTNRIARIFGGAREGAQRPRFISPASQVAICLALISVQLLIVVAWLVVEAPGTGKETAPERREVVTLRCNHRDASMLGSLAYNVLLIALCTLYAFKTRKCPENFNEAKFIGFTMYTTCIIWLAFLPIFYVTSSDYRVQTTTMCVSVSLSGSVVLGCLFAPKLHIILFQPQKNVVSHRAPTSRFGSAAARASSSLGQGSGSQFVPTVCNGREVVDSTTSSLT

SEQ ID NO: 8 - protein, 873 aa, Human
MGSLLALLALLPLWGAVAEGPAKKVLTLEGDLVLGGLFPVHQKGGPAEDCGPVNEHRGIQRLEAMLFALDRINRDPHLLPGVRLGAHILDSCSKDTHALEQALDFVRASLSRGADGSRHICPDGSYATHGDAPTAITGVIGGSYSDVSIQVANLLRLFQIPQISYASTSAKLSDKSRYDYFARTVPPDFFQAKAMAEILRFFNWTYVSTVASEGDYGETGIEAFELEARARNICVATSEKVGRAMSRAAFEGVVRALLQKPSARVAVLFTRSEDARELLAASQRLNASFTWVASDGWGALESVVAGSEGAAEGAITIELASYPISDFASYFQSLDPWNNSRNPWFREFWEQRFRCSFRQRDCAAHSLRAVPFEQESKIMFVVNAVYAMAHALHNMHRALCPNTTRLCDAMRPVNGRRLYKDFVLNVKFDAPFRPADTHNEVRFDRFGDGIGRYNIFTYLRAGSGRYRYQKVGYWAEGLTLDTSLIPWASPSAGPLPASRCSEPCLQNEVKSVQPGEVCCWLCIPCQPYEYRLDEFTCADCGLGYWPNASLTGCFELPQEYIRWGDAWAVGPVTIACLGALATLFVLGVFVRHNATPVVKASGRELCYILLGGVFLCYCMTFIFIAKPSTAVCTLRRLGLGTAFSVCYSALLTKTNRIARIFGGAREGAQRPRFISPASQVAICLALILVQLLIVVAWLVVEAPGTGKETAPERREVVTLRCNHRDASMLGSLTYDVLLIALCTLYAFKTRKCPENFNEAKFIGFTMYTTCIIWLAFLPIFYVTSSDYRVQTTTMCVSVSLSGSVVLGCLFAPKLHIILFQPQKNVVSHRAPTSRFGSAAARASSSLGQGSGSQFVPTVCNGREVVDSTTSSLT

SEQ ID NO: 9 - DNA, 2619 bp, Human
atgggatcgctgcttgcgctcctggcactgctgccgctgtggggtgctgtggctgagggcccagccaagaaggtgctgaccctggagggagacttggtgctgggtgggctgttcccagtgcaccagaagggcggcccagcagaggactgtggtcctgtcaatgagcaccgtggcatccagcgcctggaggccatgctttttgcactggaccgcatcaaccgtgacccgcacctgctgcctggcgtgcgcctgggtgcacacatcctcgacagttgctccaaggacacacatgcgctggagcaggcactggactttgtgcgtgcctcactcagccgtggtgctgatggctcacgccacatctgccccgacggctcttatgcgacccatggtgatgctcccactgccatcactggtgttattggcggttcctacagtgatgtctccatccaggtggccaacctcttgaggctatttcagatcccacagattagctacgcctctaccagtgccaagctgagtgacaagtcccgctatgactactttgcccgcacagtgcctcctgacttcttccaagccaaggccatggctgagattctccgcttcttcaactggacctatgtgtccactgtggcgtctgagggcgactatggcgagacaggcattgaggcctttgagctagaggctcgtgcccgcaacatctgtgtggccacctcggagaaagtgggccgtgccatgagccgcgcggcctttgagggtgtggtgcgagccctgctgcagaagcccagtgcccgcgtggctgtcctgttcacccgttctgaggatgcccgggagctgcttgctgccagccagcgcctcaatgccagcttcacctgggtggccagtgatggttggggggccctggagagtgtggtggcaggcagtgagggggctgctgagggtgctatcaccatcgagctggcctcctaccccatcagtgactttgcctcctacttccagagcctggacccttggaacaacagccggaacccctggttccgtgaattctgggagcagaggttccgctgcagcttccggcagcgagactgcgcagcccactctctccgggctgtgccctttgagcaggagtccaagatcatgtttgtggtcaatgcagtgtacgccatggcccatgcgctccacaacatgcaccgtgccctctgccccaacaccacccggctctgtgacgcgatgcggccagttaacgggcgccgcctctacaaggactttgtgctcaacgtcaagtttgatgccccctttcgcccagctgacacccacaatgaggtccgctttgaccgctttggtgatggtattggccgctacaacatcttcacctatctgcgtgcaggcagtgggcgctatcgctaccagaaggtgggctactgggcagaaggcttgactctggacaccagcctcatcccatgggcctcaccctcagccggccccctgcccgcctctcgctgcagtgagccctgcctccagaatgaggtgaagagtgtgcagccgggcgaagtctgctgctggctctgcattccgtgccagccctatgagtaccgattggacgaattcacttgcgctgattgtggcctgggctactggcccaatgccagcctgactggctgcttcgaactgccccaggagtacatccgctggggcgatgcctgggctgtgggacctgtcaccatcgcctgcctcggtgccctggccaccctctttgtgctgggtgtctttgtgcggcacaatgccacaccagtggtcaaggcctcaggtcgggagctctgctacatcctgctgggtggtgtcttcctctgctactgcatgaccttcatcttcattgccaagccatccacggcagtgtgtaccttacggcgtcttggtttgggcactgccttctctgtctgctactcagccctgctcaccaagaccaaccgcattgcacgcatcttcggtggggcccgggagggtgcccagcggccacgcttcatcagtcctgcctcacaggtggccatctgcctggcacttatctcgggccagctgctcatcgtggtcgcctggctggtggtggaggcaccgggcacaggcaaggagacagcccccgaacggcgggaggtggtgacactgcgctgcaaccaccgcgatgcaagtatgttgggctcgctggcctacgatgtgctcctcatcgcgctctgcacgctttatgccttcaagactcgcaagtgccccgaaaacttcaacgaggccaagttcattggcttcaccatgtacaccacctgcatcatctggctggcattcttgcccatcttctatgtcacctccagtgactaccgggtacagaccaccaccatgtgcgtgtcagtcagcctcagcggctccgtggtgcttggctgcctctttgcgcccaagctgcacatcatcctcttccagccgcagaagaacgtggttagccaccgggcacccaccagccgctttggcagtgctgctgccagggccagctccagccttggccaagggtctggctcccagtttgtccccactgtttgcaatggccgtgaggtggtggactcgacaacgtcatcgctttga

SEQ ID NO: 10 - DNA, 2619 bp, Human
atgggatcgctgcttgcgctcctggcactgctgccgctgtggggtgctgtggctgagggcccagccaagaaggtgctgaccctggagggagacttggtgctgggtgggctgttcccagtgcaccagaagggcggcccagcagaggactgtggtcctgtcaatgagcaccgtggcatccagcgcctggaggccatgctttttgcactggaccgcatcaaccgtgacccgcacctgctgcctggcgtgcgcctgggtgcacacatcctcgacagttgctccaaggacacacatgcgctggagcaggcactggactttgtgcgtgcctcactcagccgtggtgctgatggctcacgccacatctgccccgacggctcttatgcgacccatggtgatgctcccactgccatcactggtgttattggcggttcctacagtgatgtctccatccaggtggccaacctcttgaggctatttcagatcccacagattagctacgcctctaccagtgccaagctgagtgacaagtcccgctatgactactttgcccgcacagtgcctcctgacttcttccaagccaaggccatggctgagattctccgcttcttcaactggacctatgtgtccactgtggcgtctgagggcgactatggcgagacaggcattgaggcctttgagctagaggctcgtgcccgcaacatctgtgtggccacctcggagaaagtgggccgtgccatgagccgcgcggcctttgagggtgtggtgcgagccctgctgcagaagcccagtgcccgcgtggctgtcctgttcacccgttctgaggatgcccgggagctgcttgctgccagccagcgcctcaatgccagcttcacctgggtggccagtgatggttggggggccctggagagtgtggtggcaggcagtgagggggctgctgagggtgctatcaccatcgagctggcctcctaccccatcagtgactttgcctcctacttccagagcctggacccttggaacaacagccggaacccctggttccgtgaattctgggagcagaggttccgctgcagcttccggcagcgagactgcgcagcccactctctccgggctgtgccctttgagcaggagtccaagatcatgtttgtggtcaatgcagtgtacgccatggcccatgcgctccacaacatgcaccgtgccctctgccccaacaccacccggctctgtgacgcgatgcggccagttaacgggcgccgcctctacaaggactttgtgctcaacgtcaagtttgatgccccctttcgcccagctgacacccacaatgaggtccgctttgaccgctttggtgatggtattggccgctacaacatcttcacctatctgcgtgcaggcagtgggcgctatcgctaccagaaggtgggctactgggcagaaggcttgactctggacaccagcctcatcccatgggcctcaccctcagccggccccctgcccgcctctcgctgcagtgagccctgcctccagaatgaggtgaagagtgtgcagccgggcgaagtctgctgctggctctgcattccgtgccagccctatgagtaccgattggacgaattcacttgcgctgattgtggcctgggctactggcccaatgccagcctgactggctgcttcgaactgccccaggagtacatccgctggggcgatgcctgggctgtgggacctgtcaccatcgcctgcctcggtgccctggccaccctctttgtgctgggtgtctttgtgcggcacaatgccacaccagtggtcaaggcctcaggtcgggagctctgctacatcctgctgggtggtgtcttcctctgctactgcatgaccttcatcttcattgccaagccatccacggcagtgtgtaccttacggcgtcttggtttgggcactgccttctctgtctgctactcagccctgctcaccaagaccaaccgcattgcacgcatcttcggtggggcccgggagggtgcccagcggccacgcttcatcagtcctgcctcacaggtggccatctgcctggcacttatctcggtccagctgctcatcgtggtcgcctggctggtggtggaggcaccgggcacaggcaaggagacagcccccgaacggcgggaggtggtgacactgcgctgcaaccaccgcgatgcaagtatgttgggctcgctggcctacgatgtgctcctcatcgcgctctgcacgctttatgccttcaagactcgcaagtgccccgaaaacttcaacgaggccaagttcattggcttcaccatgtacaccacctgcatcatctggctggcattcttgcccatcttctatgtcacctccagtgactaccgggtacagaccaccaccatgtgcgtgtcagtcagcctcagcggctccgtggtgcttggctgcctctttgcgcccaagctgcacatcatcctcttccagccgcagaagaacgtggttagccaccgggcacccaccagccgctttggcagtgctgctgccagggccagctccagccttggccaagggtctggctcccagtttgtccccactgtttgcaatggccgtgaggtggtggactcgacaacgtcatcgctttga

SEQ ID NO: 11 - DNA, 2619 bp, Human
atgggatcgctgcttgcgctcctggcactgctgccgctgtggggtgctgtggctgagggcccagccaagaaggtgctgaccctggagggagacttggtgctgggtgggctgttcccagtgcaccagaagggcggcccagcagaggactgtggtcctgtcaatgagcaccgtggcatccagcgcctggaggccatgctttttgcactggaccgcatcaaccgtgacccgcacctgctgcctggcgtgcgcctgggtgcacacatcctcgacagttgctccaaggacacacatgcgctggagcaggcactggactttgtgcgtgcctcactcagccgtggtgctgatggctcacgccacatctgccccgacggctcttatgcgacccatggtgatgctcccactgccatcactggtgttattggcggttcctacagtgatgtctccatccaggtggccaacctcttgaggctatttcagatcccacagattagctacgcctctaccagtgccaagctgagtgacaagtcccgctatgactactttgcccgcacagtgcctcctgacttcttccaagccaaggccatggctgagattctccgcttcttcaactggacctatgtgtccactgtggcgtctgagggcgactatggcgagacaggcattgaggcctttgagctagaggctcgtgcccgcaacatctgtgtggccacctcggagaaagtgggccgtgccatgagccgcgcggcctttgagggtgtggtgcgagccctgctgcagaagcccagtgcccgcgtggctgtcctgttcacccgttctgaggatgcccgggagctgcttgctgccagccagcgcctcaatgccagcttcacctgggtggccagtgatggttggggggccctggagagtgtggtggcaggcagtgagggggctgctgagggtgctatcaccatcgagctggcctcctaccccatcagtgactttgcctcctacttccagagcctggacccttggaacaacagccggaacccctggttccgtgaattctgggagcagaggttccgctgcagcttccggcagcgagactgcgcagcccactctctccgggctgtgccctttgagcaggagtccaagatcatgtttgtggtcaatgcagtgtacgccatggcccatgcgctccacaacatgcaccgtgccctctgccccaacaccacccggctctgtgacgcgatgcggccagttaacgggcgccgcctctacaaggactttgtgctcaacgtcaagtttgatgccccctttcgcccagctgacacccacaatgaggtccgctttgaccgctttggtgatggtattggccgctacaacatcttcacctatctgcgtgcaggcagtgggcgctatcgctaccagaaggtgggctactgggcagaaggcttgactctggacaccagcctcatcccatgggcctcaccctcagccggccccctgcccgcctctcgctgcagtgagccctgcctccagaatgaggtgaagagtgtgcagccgggcgaagtctgctgctggctctgcattccgtgccagccctatgagtaccgattggacgaattcacttgcgctgattgtggcctgggctactggcccaatgccagcctgactggctgcttcgaactgccccaggagtacatccgctggggcgatgcctgggctgtgggacctgtcaccatcgcctgcctcggtgccctggccaccctctttgtgctgggtgtctttgtgcggcacaatgccacaccagtggtcaaggcctcaggtcgggagctctgctacatcctgctgggtggtgtcttcctctgctactgcatgaccttcatcttcattgccaagccatccacggcagtgtgtaccttacggcgtcttggtttgggcactgccttctctgtctgctactcagccctgctcaccaagaccaaccgcattgcacgcatcttcggtggggcccgggagggtgcccagcggccacgcttcatcagtcctgcctcacaggtggccatctgcctggcacttatcttggtccagctgctcatcgtggtcgcctggctggtggtggaggcaccgggcacaggcaaggagacagcccccgaacggcgggaggtggtgacactgcgctgcaaccaccgcgatgcaagtatgttgggctcgctggcctacgatgtgctcctcatcgcgctctgcacgctttatgccttcaagactcgcaagtgccccgaaaacttcaacgaggccaagttcattggcttcaccatgtacaccacctgcatcatctggctggcattcttgcccatcttctatgtcacctccagtgactaccgggtacagaccaccaccatgtgcgtgtcagtcagcctcagcggctccgtggtgcttggctgcctctttgcgcccaagctgcacatcatcctcttccagccgcagaagaacgtggttagccaccgggcacccaccagccgctttggcagtgctgctgccagggccagctccagccttggccaagggtctggctcccagtttgtccccactgtttgcaatggccgtgaggtggtggactcgacaacgtcatcgctttga

SEQ ID NO: 12 - DNA, 2619 bp, Human
atgggatcgctgcttgcgctcctggcactgctgccgctgtggggtgctgtggctgagggcccagccaagaaggtgctgaccctggagggagacttggtgctgggtgggctgttcccagtgcaccagaagggcggcccagcagaggactgtggtcctgtcaatgagcaccgtggcatccagcgcctggaggccatgctttttgcactggaccgcatcaaccgtgacccgcacctgctgcctggcgtgcgcctgggtgcacacatcctcgacagttgctccaaggacacacatgcgctggagcaggcactggactttgtgcgtgcctcactcagccgtggtgctgatggctcacgccacatctgccccgacggctcttatgcgacccatggtgatgctcccactgccatcactggtgttattggcggttcctacagtgatgtctccatccaggtggccaacctcttgaggctatttcagatcccacagattagctacgcctctaccagtgccaagctgagtgacaagtcccgctatgactactttgcccgcacagtgcctcctgacttcttccaagccaaggccatggctgagattctccgcttcttcaactggacctatgtgtccactgtggcgtctgagggcgactatggcgagacaggcattgaggcctttgagctagaggctcgtgcccgcaacatctgtgtggccacctcggagaaagtgggccgtgccatgagccgcgcggcctttgagggtgtggtgcgagccctgctgcagaagcccagtgcccgcgtggctgtcctgttcacccgttctgaggatgcccgggagctgcttgctgccagccagcgcctcaatgccagcttcacctgggtggccagtgatggttggggggccctggagagtgtggtggcaggcagtgagggggctgctgagggtgctatcaccatcgagctggcctcctaccccatcagtgactttgcctcctacttccagagcctggacccttggaacaacagccggaacccctggttccgtgaattctgggagcagaggttccgctgcagcttccggcagcgagactgcgcagcccactctctccgggctgtgccctttgagcaggagtccaagatcatgtttgtggtcaatgcagtgtacgccatggcccatgcgctccacaacatgcaccgtgccctctgccccaacaccacccggctctgtgacgcgatgcggccagttaacgggcgccgcctctacaaggactttgtgctcaacgtcaagtttgatgccccctttcgcccagctgacacccacaatgaggtccgctttgaccgctttggtgatggtattggccgctacaacatcttcacctatctgcgtgcaggcagtgggcgctatcgctaccagaaggtgggctactgggcagaaggcttgactctggacaccagcctcatcccatgggcctcaccctcagccggccccctgcccgcctctcgctgcagtgagccctgcctccagaatgaggtgaagagtgtgcagccgggcgaagtctgctgctggctctgcattccgtgccagccctatgagtaccgattggacgaattcacttgcgctgattgtggcctgggctactggcccaatgccagcctgactggctgcttcgaactgccccaggagtacatccgctggggcgatgcctgggctgtgggacctgtcaccatcgcctgcctcggtgccctggccaccctctttgtgctgggtgtctttgtgcggcacaatgccacaccagtggtcaaggcctcaggtcgggagctctgctacatcctgctgggtggtgtcttcctctgctactgcatgaccttcatcttcattgccaagccatccacggcagtgtgtaccttacggcgtcttggtttgggcactgccttctctgtctgctactcagccctgctcaccaagaccaaccgcattgcacgcatcttcggtggggcccgggagggtgcccagcggccacgcttcatcagtcctgcctcacaggtggccatctgcctggcacttatcttgggccagctgctcatcgtggtcgcctggctggtggtggaggcaccgggcacaggcaaggagacagcccccgaacggcgggaggtggtgacactgcgctgcaaccaccgcgatgcaagtatgttgggctcgctggcctacaatgtgctcctcatcgcgctctgcacgctttatgccttcaagactcgcaagtgccccgaaaacttcaacgaggccaagttcattggcttcaccatgtacaccacctgcatcatctggctggcattcttgcccatcttctatgtcacctccagtgactaccgggtacagaccaccaccatgtgcgtgtcagtcagcctcagcggctccgtggtgcttggctgcctctttgcgcccaagctgcacatcatcctcttccagccgcagaagaacgtggttagccaccgggcacccaccagccgctttggcagtgctgctgccagggccagctccagccttggccaagggtctggctcccagtttgtccccactgtttgcaatggccgtgaggtggtggactcgacaacgtcatcgctttga

SEQ ID NO: 13 - DNA, 2619 bp, Human
atgggatcgctgcttgcgctcctggcactgctgccgctgtggggtgctgtggctgagggcccagccaagaaggtgctgaccctggagggagacttggtgctgggtgggctgttcccagtgcaccagaagggcggcccagcagaggactgtggtcctgtcaatgagcaccgtggcatccagcgcctggaggccatgctttttgcactggaccgcatcaaccgtgacccgcacctgctgcctggcgtgcgcctgggtgcacacatcctcgacagttgctccaaggacacacatgcgctggagcaggcactggactttgtgcgtgcctcactcagccgtggtgctgatggctcacgccacatctgccccgacggctcttatgcgacccatggtgatgctcccactgccatcactggtgttattggcggttcctacagtgatgtctccatccaggtggccaacctcttgaggctatttcagatcccacagattagctacgcctctaccagtgccaagctgagtgacaagtcccgctatgactactttgcccgcacagtgcctcctgacttcttccaagccaaggccatggctgagattctccgcttcttcaactggacctatgtgtccactgtggcgtctgagggcgactatggcgagacaggcattgaggcctttgagctagaggctcgtgcccgcaacatctgtgtggccacctcggagaaagtgggccgtgccatgagccgcgcggcctttgagggtgtggtgcgagccctgctgcagaagcccagtgcccgcgtggctgtcctgttcacccgttctgaggatgcccgggagctgcttgctgccagccagcgcctcaatgccagcttcacctgggtggccagtgatggttggggggccctggagagtgtggtggcaggcagtgagggggctgctgagggtgctatcaccatcgagctggcctcctaccccatcagtgactttgcctcctacttccagagcctggacccttggaacaacagccggaacccctggttccgtgaattctgggagcagaggttccgctgcagcttccggcagcgagactgcgcagcccactctctccgggctgtgccctttgagcaggagtccaagatcatgtttgtggtcaatgcagtgtacgccatggcccatgcgctccacaacatgcaccgtgccctctgccccaacaccacccggctctgtgacgcgatgcggccagttaacgggcgccgcctctacaaggactttgtgctcaacgtcaagtttgatgccccctttcgcccagctgacacccacaatgaggtccgctttgaccgctttggtgatggtattggccgctacaacatcttcacctatctgcgtgcaggcagtgggcgctatcgctaccagaaggtgggctactgggcagaaggcttgactctggacaccagcctcatcccatgggcctcaccctcagccggccccctgcccgcctctcgctgcagtgagccctgcctccagaatgaggtgaagagtgtgcagccgggcgaagtctgctgctggctctgcattccgtgccagccctatgagtaccgattggacgaattcacttgcgctgattgtggcctgggctactggcccaatgccagcctgactggctgcttcgaactgccccaggagtacatccgctggggcgatgcctgggctgtgggacctgtcaccatcgcctgcctcggtgccctggccaccctctttgtgctgggtgtctttgtgcggcacaatgccacaccagtggtcaaggcctcaggtcgggagctctgctacatcctgctgggtggtgtcttcctctgctactgcatgaccttcatcttcattgccaagccatccacggcagtgtgtaccttacggcgtcttggtttgggcactgccttctctgtctgctactcagccctgctcaccaagaccaaccgcattgcacgcatcttcggtggggcccgggagggtgcccagcggccacgcttcatcagtcctgcctcacaggtggccatctgcctggcacttatcttggtccagctgctcatcgtggtcgcctggctggtggtggaggcaccgggcacaggcaaggagacagcccccgaacggcgggaggtggtgacactgcgctgcaaccaccgcgatgcaagtatgttgggctcgctggcctacaatgtgctcctcatcgcgctctgcacgctttatgccttcaagactcgcaagtgccccgaaaacttcaacgaggccaagttcattggcttcaccatgtacaccacctgcatcatctggctggcattcttgcccatcttctatgtcacctccagtgactaccgggtacagaccaccaccatgtgcgtgtcagtcagcctcagcggctccgtggtgcttggctgcctctttgcgcccaagctgcacatcatcctcttccagccgcagaagaacgtggttagccaccgggcacccaccagccgctttggcagtgctgctgccagggccagctccagccttggccaagggtctggctcccagtttgtccccactgtttgcaatggccgtgaggtggtggactcgacaacgtcatcgctttga

SEQ ID NO: 14 - DNA, 2619 bp, Human
atgggatcgctgcttgcgctcctggcactgctgccgctgtggggtgctgtggctgagggcccagccaagaaggtgctgaccctggagggagacttggtgctgggtgggctgttcccagtgcaccagaagggcggcccagcagaggactgtggtcctgtcaatgagcaccgtggcatccagcgcctggaggccatgctttttgcactggaccgcatcaaccgtgacccgcacctgctgcctggcgtgcgcctgggtgcacacatcctcgacagttgctccaaggacacacatgcgctggagcaggcactggactttgtgcgtgcctcactcagccgtggtgctgatggctcacgccacatctgccccgacggctcttatgcgacccatggtgatgctcccactgccatcactggtgttattggcggttcctacagtgatgtctccatccaggtggccaacctcttgaggctatttcagatcccacagattagctacgcctctaccagtgccaagctgagtgacaagtcccgctatgactactttgcccgcacagtgcctcctgacttcttccaagccaaggccatggctgagattctccgcttcttcaactggacctatgtgtccactgtggcgtctgagggcgactatggcgagacaggcattgaggcctttgagctagaggctcgtgcccgcaacatctgtgtggccacctcggagaaagtgggccgtgccatgagccgcgcggcctttgagggtgtggtgcgagccctgctgcagaagcccagtgcccgcgtggctgtcctgttcacccgttctgaggatgcccgggagctgcttgctgccagccagcgcctcaatgccagcttcacctgggtggccagtgatggttggggggccctggagagtgtggtggcaggcagtgagggggctgctgagggtgctatcaccatcgagctggcctcctaccccatcagtgactttgcctcctacttccagagcctggacccttggaacaacagccggaacccctggttccgtgaattctgggagcagaggttccgctgcagcttccggcagcgagactgcgcagcccactctctccgggctgtgccctttgagcaggagtccaagatcatgtttgtggtcaatgcagtgtacgccatggcccatgcgctccacaacatgcaccgtgccctctgccccaacaccacccggctctgtgacgcgatgcggccagttaacgggcgccgcctctacaaggactttgtgctcaacgtcaagtttgatgccccctttcgcccagctgacacccacaatgaggtccgctttgaccgctttggtgatggtattggccgctacaacatcttcacctatctgcgtgcaggcagtgggcgctatcgctaccagaaggtgggctactgggcagaaggcttgactctggacaccagcctcatcccatgggcctcaccctcagccggccccctgcccgcctctcgctgcagtgagccctgcctccagaatgaggtgaagagtgtgcagccgggcgaagtctgctgctggctctgcattccgtgccagccctatgagtaccgattggacgaattcacttgcgctgattgtggcctgggctactggcccaatgccagcctgactggctgcttcgaactgccccaggagtacatccgctggggcgatgcctgggctgtgggacctgtcaccatcgcctgcctcggtgccctggccaccctctttgtgctgggtgtctttgtgcggcacaatgccacaccagtggtcaaggcctcaggtcgggagctctgctacatcctgctgggtggtgtcttcctctgctactgcatgaccttcatcttcattgccaagccatccacggcagtgtgtaccttacggcgtcttggtttgggcactgccttctctgtctgctactcagccctgctcaccaagaccaaccgcattgcacgcatcttcggtggggcccgggagggtgcccagcggccacgcttcatcagtcctgcctcacaggtggccatctgcctggcacttatcttgggccagctgctcatcgtggtcgcctggctggtggtggaggcaccgggcacaggcaaggagacagcccccgaacggcgggaggtggtgacactgcgctgcaaccaccgcgatgcaagtatgttgggctcgctggcctacgatgtgctcctcatcgcgctctgcacgctttatgccttcaagactcgcaagtgccccgaaaacttcaacgaggccaagttcattggcttcaccatgtacaccacctgcatcatctggctggcattcttgcccatcttctatgtcacctccagtgactaccgggtacagaccaccaccatgtgcgtgtcagtcagcctcagcggctccgtggtgcttggctgcctctttgcgcccaagctgcacatcatcctcttccagccgcagaagaacgtggttagccaccgggcacccaccagccgctttggcagtgctgctgccagggccagctccagccttggccaagggtctggctcccagtttgtccccactgtttgcaatggccgtgaggtggtggactcgacaacgtcatcgctttga

SEQ ID NO: 15 - DNA, 2619 bp, Human
atgggatcgctgcttgcgctcctggcactgctgccgctgtggggtgctgtggctgagggcccagccaagaaggtgctgaccctggagggagacttggtgctgggtgggctgttcccagtgcaccagaagggcggcccagcagaggactgtggtcctgtcaatgagcaccgtggcatccagcgcctggaggccatgctttttgcactggaccgcatcaaccgtgacccgcacctgctgcctggcgtgcgcctgggtgcacacatcctcgacagttgctccaaggacacacatgcgctggagcaggcactggactttgtgcgtgcctcactcagccgtggtgctgatggctcacgccacatctgccccgacggctcttatgcgacccatggtgatgctcccactgccatcactggtgttattggcggttcctacagtgatgtctccatccaggtggccaacctcttgaggctatttcagatcccacagattagctacgcctctaccagtgccaagctgagtgacaagtcccgctatgactactttgcccgcacagtgcctcctgacttcttccaagccaaggccatggctgagattctccgcttcttcaactggacctatgtgtccactgtggcgtctgagggcgactatggcgagacaggcattgaggcctttgagctagaggctcgtgcccgcaacatctgtgtggccacctcggagaaagtgggccgtgccatgagccgcgcggcctttgagggtgtggtgcgagccctgctgcagaagcccagtgcccgcgtggctgtcctgttcacccgttctgaggatgcccgggagctgcttgctgccagccagcgcctcaatgccagcttcacctgggtggccagtgatggttggggggccctggagagtgtggtggcaggcagtgagggggctgctgagggtgctatcaccatcgagctggcctcctaccccatcagtgactttgcctcctacttccagagcctggacccttggaacaacagccggaacccctggttccgtgaattctgggagcagaggttccgctgcagcttccggcagcgagactgcgcagcccactctctccgggctgtgccctttgagcaggagtccaagatcatgtttgtggtcaatgcagtgtacgccatggcccatgcgctccacaacatgcaccgtgccctctgccccaacaccacccggctctgtgacgcgatgcggccagttaacgggcgccgcctctacaaggactttgtgctcaacgtcaagtttgatgccccctttcgcccagctgacacccacaatgaggtccgctttgaccgctttggtgatggtattggccgctacaacatcttcacctatctgcgtgcaggcagtgggcgctatcgctaccagaaggtgggctactgggcagaaggcttgactctggacaccagcctcatcccatgggcctcaccctcagccggccccctgcccgcctctcgctgcagtgagccctgcctccagaatgaggtgaagagtgtgcagccgggcgaagtctgctgctggctctgcattccgtgccagccctatgagtaccgattggacgaattcacttgcgctgattgtggcctgggctactggcccaatgccagcctgactggctgcttcgaactgccccaggagtacatccgctggggcgatgcctgggctgtgggacctgtcaccatcgcctgcctcggtgccctggccaccctctttgtgctgggtgtctttgtgcggcacaatgccacaccagtggtcaaggcctcaggtcgggagctctgctacatcctgctgggtggtgtcttcctctgctactgcatgaccttcatcttcattgccaagccatccacggcagtgtgtaccttacggcgtcttggtttgggcactgccttctctgtctgctactcagccctgctcaccaagaccaaccgcattgcacgcatcttcggtggggcccgggagggtgcccagcggccacgcttcatcagtcctgcctcacaggtggccatctgcctggcacttatctcggtccagctgctcatcgtggtcgcctggctggtggtggaggcaccgggcacaggcaaggagacagcccccgaacggcgggaggtggtgacactgcgctgcaaccaccgcgatgcaagtatgttgggctcgctggcctacaatgtgctcctcatcgcgctctgcacgctttatgccttcaagactcgcaagtgccccgaaaacttcaacgaggccaagttcattggcttcaccatgtacaccacctgcatcatctggctggcattcttgcccatcttctatgtcacctccagtgactaccgggtacagaccaccaccatgtgcgtgtcagtcagcctcagcggctccgtggtgcttggctgcctctttgcgcccaagctgcacatcatcctcttccagccgcagaagaacgtggttagccaccgggcacccaccagccgctttggcagtgctgctgccagggccagctccagccttggccaagggtctggctcccagtttgtccccactgtttgcaatggccgtgaggtggtggactcgacaacgtcatcgctttga

SEQ ID NO: 16 - DNA, 2619 bp, Human
atgggatcgctgcttgcgctcctggcactgctgccgctgtggggtgctgtggctgagggcccagccaagaaggtgctgaccctggagggagacttggtgctgggtgggctgttcccagtgcaccagaagggcggcccagcagaggactgtggtcctgtcaatgagcaccgtggcatccagcgcctggaggccatgctttttgcactggaccgcatcaaccgtgacccgcacctgctgcctggcgtgcgcctgggtgcacacatcctcgacagttgctccaaggacacacatgcgctggagcaggcactggactttgtgcgtgcctcactcagccgtggtgctgatggctcacgccacatctgccccgacggctcttatgcgacccatggtgatgctcccactgccatcactggtgttattggcggttcctacagtgatgtctccatccaggtggccaacctcttgaggctatttcagatcccacagattagctacgcctctaccagtgccaagctgagtgacaagtcccgctatgactactttgcccgcacagtgcctcctgacttcttccaagccaaggccatggctgagattctccgcttcttcaactggacctatgtgtccactgtggcgtctgagggcgactatggcgagacaggcattgaggcctttgagctagaggctcgtgcccgcaacatctgtgtggccacctcggagaaagtgggccgtgccatgagccgcgcggcctttgagggtgtggtgcgagccctgctgcagaagcccagtgcccgcgtggctgtcctgttcacccgttctgaggatgcccgggagctgcttgctgccagccagcgcctcaatgccagcttcacctgggtggccagtgatggttggggggccctggagagtgtggtggcaggcagtgagggggctgctgagggtgctatcaccatcgagctggcctcctaccccatcagtgactttgcctcctacttccagagcctggacccttggaacaacagccggaacccctggttccgtgaattctgggagcagaggttccgctgcagcttccggcagcgagactgcgcagcccactctctccgggctgtgccctttgagcaggagtccaagatcatgtttgtggtcaatgcagtgtacgccatggcccatgcgctccacaacatgcaccgtgccctctgccccaacaccacccggctctgtgacgcgatgcggccagttaacgggcgccgcctctacaaggactttgtgctcaacgtcaagtttgatgccccctttcgcccagctgacacccacaatgaggtccgctttgaccgctttggtgatggtattggccgctacaacatcttcacctatctgcgtgcaggcagtgggcgctatcgctaccagaaggtgggctactgggcagaaggcttgactctggacaccagcctcatcccatgggcctcaccctcagccggccccctgcccgcctctcgctgcagtgagccctgcctccagaatgaggtgaagagtgtgcagccgggcgaagtctgctgctggctctgcattccgtgccagccctatgagtaccgattggacgaattcacttgcgctgattgtggcctgggctactggcccaatgccagcctgactggctgcttcgaactgccccaggagtacatccgctggggcgatgcctgggctgtgggacctgtcaccatcgcctgcctcggtgccctggccaccctctttgtgctgggtgtctttgtgcggcacaatgccacaccagtggtcaaggcctcaggtcgggagctctgctacatcctgctgggtggtgtcttcctctgctactgcatgaccttcatcttcattgccaagccatccacggcagtgtgtaccttacggcgtcttggtttgggcactgccttctctgtctgctactcagccctgctcaccaagaccaaccgcattgcacgcatcttcggtggggcccgggagggtgcccagcggccacgcttcatcagtcctgcctcacaggtggccatctgcctggcacttatcttggtccagctgctcatcgtggtcgcctggctggtggtggaggcaccgggcacaggcaaggagacagcccccgaacggcgggaggtggtgacactgcgctgcaaccaccgcgatgcaagtatgttgggctcgctgacctacgatgtgctcctcatcgcgctctgcacgctttatgccttcaagactcgcaagtgccccgaaaacttcaacgaggccaagttcattggcttcaccatgtacaccacctgcatcatctggctggcattcttgcccatcttctatgtcacctccagtgactaccgggtacagaccaccaccatgtgcgtgtcagtcagcctcagcggctccgtggtgcttggctgcctctttgcgcccaagctgcacatcatcctcttccagccgcagaagaacgtggttagccaccgggcacccaccagccgctttggcagtgctgctgccagggccagctccagccttggccaagggtctggctcccagtttgtccccactgtttgcaatggccgtgaggtggtggactcgacaacgtcatcgctttga

SEQ ID NO: 17 - protein, 873 aa, Rat
MESLLGFLALLLLWGAVAEGPAKKVLTLEGDLVLGGLFPVHQKGGPAEECGPVNEHRGIQRLEAMLFALDRINRDPHLLPGVRLGAHILDSCSKDTHALEQALDFVRASLSRGADGSRHICPDGSYATHSDAPTAVTGVIGGSYSDVSIQVANLLRLFQIPQISYASTSAKLSDKSRYDYFARTVPPDFFQAKAMAEILRFFNWTYVSTVASEGDYGETGIEAFELEARARNICVATSEKVGRAMSRAAFEGVVRALLQKPSARVAVLFTRSEDARELLAATQRLNASFTWVASDGWGALESVVAGSERAAEGAITIELASYPISDFASYFQSLDPWNNSRNPWFREFWEERFHCSFRQRDCAAHSLRAVPFEQESKIMFVVNAVYAMAHALHNMHRALCPNTTHLCDAMRPVNGRRLYKDFVLNVKFDAPFRPADTDDEVRFDRFGDGIGRYNIFTYLRAGSGRYRYQKVGYWAEGLTLDTSFIPWASPSAGPLPASRCSEPCLQNEVKSVQPGEVCCWLCIPCQPYEYRLDEFTCADCGLGYWPNASLTGCFELPQEYIRWGDAWAVGPVTIACLGALATLFVLGVFVRHNATPVVKASGRELCYILLGGVFLCYCMTFVFIAKPSTAVCTLRRLGLGTAFSVCYSALLTKTNRIARIFGGAREGAQRPRFISPASQVAICLALISGQLLIVAAWLVVEAPGTGKETAPERREVVTLRCNHRDASMLGSLAYDVLLIALCTLYAFKTRKCPENFNEAKFIGFTMYTTCIIWLAFLPIFYVTSSDYRVQTTTMCVSVSLSGSVVLGCLFAPKLHIILFQPQKNVVSHRAPTSRFGSAAPRASANLGQGSGSQFVPTVCNGREVVDSTTSSLT

SEQ ID NO: 18 - protein, 873 aa, Rat
MESLLGFLALLLLWGAVAEGPAKKVLTLEGDLVLGGLFPVHQKGGPAEECGPVNEHRGIQRLEAMLFALDRINRDPHLLPGVRLGAHILDSCSKDTHALEQALDFVRASLSRGADGSRHICPDGSYATHSDAPTAVTGVIGGSYSDVSIQVANLLRLFQIPQISYASTSAKLSDKSRYDYFARTVPPDFFQAKAMAEILRFFNWTYVSTVASEGDYGETGIEAFELEARARNICVATSEKVGRAMSRAAFEGVVRALLQKPSARVAVLFTRSEDARELLAATQRLNASFTWVASDGWGALESVVAGSERAAEGAITIELASYPISDFASYFQSLDPWNNSRNPWFREFWEERFHCSFRQRDCAAHSLRAVPFEQESKIMFVVNAVYAMAHALHNMHRALCPNTTHLCDAMRPVNGRRLYKDFVLNVKFDAPFRPADTDDEVRFDRFGDGIGRYNIFTYLRAGSGRYRYQKVGYWAEGLTLDTSFIPWASPSAGPLPASRCSEPCLQNEVKSVQPGEVCCWLCIPCQPYEYRLDEFTCADCGLGYWPNASLTGCFELPQEYIRWGDAWAVGPVTIACLGALATLFVLGVFVRHNATPVVKASGRELCYILLGGVFLCYCMTFVFIAKPSTAVCTLRRLGLGTAFSVCYSALLTKTNRIARIFGGAREGAQRPRFISPASQVAICLALISVQLLIVAAWLVVEAPGTGKETAPERREVVTLRCNHRDASMLGSLAYDVLLIALCTLYAFKTRKCPENFNEAKFIGFTMYTTCIIWLAFLPIFYVTSSDYRVQTTTMCVSVSLSGSVVLGCLFAPKLHIILFQPQKNVVSHRAPTSRFGSAAPRASANLGQGSGSQFVPTVCNGREVVDSTTSSLT

SEQ ID NO: 19 - protein, 873 aa, Rat
MESLLGFLALLLLWGAVAEGPAKKVLTLEGDLVLGGLFPVHQKGGPAEECGPVNEHRGIQRLEAMLFALDRINRDPHLLPGVRLGAHILDSCSKDTHALEQALDFVRASLSRGADGSRHICPDGSYATHSDAPTAVTGVIGGSYSDVSIQVANLLRLFQIPQISYASTSAKLSDKSRYDYFARTVPPDFFQAKAMAEILRFFNWTYVSTVASEGDYGETGIEAFELEARARNICVATSEKVGRAMSRAAFEGVVRALLQKPSARVAVLFTRSEDARELLAATQRLNASFTWVASDGWGALESVVAGSERAAEGAITIELASYPISDFASYFQSLDPWNNSRNPWFREFWEERFHCSFRQRDCAAHSLRAVPFEQESKIMFVVNAVYAMAHALHNMHRALCPNTTHLCDAMRPVNGRRLYKDFVLNVKFDAPFRPADTDDEVRFDRFGDGIGRYNIFTYLRAGSGRYRYQKVGYWAEGLTLDTSFIPWASPSAGPLPASRCSEPCLQNEVKSVQPGEVCCWLCIPCQPYEYRLDEFTCADCGLGYWPNASLTGCFELPQEYIRWGDAWAVGPVTIACLGALATLFVLGVFVRHNATPVVKASGRELCYILLGGVFLCYCMTFVFIAKPSTAVCTLRRLGLGTAFSVCYSALLTKTNRIARIFGGAREGAQRPRFISPASQVAICLALILVQLLIVAAWLVVEAPGTGKETAPERREVVTLRCNHRDASMLGSLAYDVLLIALCTLYAFKTRKCPENFNEAKFIGFTMYTTCIIWLAFLPIFYVTSSDYRVQTTTMCVSVSLSGSVVLGCLFAPKLHIILFQPQKNVVSHRAPTSRFGSAAPRASANLGQGSGSQFVPTVCNGREVVDSTTSSLT

SEQ ID NO: 20 - protein, 873 aa, Rat
MESLLGFLALLLLWGAVAEGPAKKVLTLEGDLVLGGLFPVHQKGGPAEECGPVNEHRGIQRLEAMLFALDRINRDPHLLPGVRLGAHILDSCSKDTHALEQALDFVRASLSRGADGSRHICPDGSYATHSDAPTAVTGVIGGSYSDVSIQVANLLRLFQIPQISYASTSAKLSDKSRYDYFARTVPPDFFQAKAMAEILRFFNWTYVSTVASEGDYGETGIEAFELEARARNICVATSEKVGRAMSRAAFEGVVRALLQKPSARVAVLFTRSEDARELLAATQRLNASFTWVASDGWGALESVVAGSERAAEGAITIELASYPISDFASYFQSLDPWNNSRNPWFREFWEERFHCSFRQRDCAAHSLRAVPFEQESKIMFVVNAVYAMAHALHNMHRALCPNTTHLCDAMRPVNGRRLYKDFVLNVKFDAPFRPADTDDEVRFDRFGDGIGRYNIFTYLRAGSGRYRYQKVGYWAEGLTLDTSFIPWASPSAGPLPASRCSEPCLQNEVKSVQPGEVCCWLCIPCQPYEYRLDEFTCADCGLGYWPNASLTGCFELPQEYIRWGDAWAVGPVTIACLGALATLFVLGVFVRHNATPVVKASGRELCYILLGGVFLCYCMTFVFIAKPSTAVCTLRRLGLGTAFSVCYSALLTKTNRIARIFGGAREGAQRPRFISPASQVAICLALILGQLLIVAAWLVVEAPGTGKETAPERREVVTLRCNHRDASMLGSLAYNVLLIALCTLYAFKTRKCPENFNEAKFIGFTMYTTCIIWLAFLPIFYVTSSDYRVQTTTMCVSVSLSGSVVLGCLFAPKLHIILFQPQKNVVSHRAPTSRFGSAAPRASANLGQGSGSQFVPTVCNGREVVDSTTSSLT

SEQ ID NO: 21 - protein, 873 aa, Rat
MESLLGFLALLLLWGAVAEGPAKKVLTLEGDLVLGGLFPVHQKGGPAEECGPVNEHRGIQRLEAMLFALDRINRDPHLLPGVRLGAHILDSCSKDTHALEQALDFVRASLSRGADGSRHICPDGSYATHSDAPTAVTGVIGGSYSDVSIQVANLLRLFQIPQISYASTSAKLSDKSRYDYFARTVPPDFFQAKAMAEILRFFNWTYVSTVASEGDYGETGIEAFELEARARNICVATSEKVGRAMSRAAFEGVVRALLQKPSARVAVLFTRSEDARELLAATQRLNASFTWVASDGWGALESVVAGSERAAEGAITIELASYPISDFASYFQSLDPWNNSRNPWFREFWEERFHCSFRQRDCAAHSLRAVPFEQESKIMFVVNAVYAMAHALHNMHRALCPNTTHLCDAMRPVNGRRLYKDFVLNVKFDAPFRPADTDDEVRFDRFGDGIGRYNIFTYLRAGSGRYRYQKVGYWAEGLTLDTSFIPWASPSAGPLPASRCSEPCLQNEVKSVQPGEVCCWLCIPCQPYEYRLDEFTCADCGLGYWPNASLTGCFELPQEYIRWGDAWAVGPVTIACLGALATLFVLGVFVRHNATPVVKASGRELCYILLGGVFLCYCMTFVFIAKPSTAVCTLRRLGLGTAFSVCYSALLTKTNRIARIFGGAREGAQRPRFISPASQVAICLALILVQLLIVAAWLVVEAPGTGKETAPERREVVTLRCNHRDASMLGSLAYNVLLIALCTLYAFKTRKCPENFNEAKFIGFTMYTTCIIWLAFLPIFYVTSSDYRVQTTTMCVSVSLSGSVVLGCLFAPKLHIILFQPQKNVVSHRAPTSRFGSAAPRASANLGQGSGSQFVPTVCNGREVVDSTTSSLT

SEQ ID NO: 22 - protein, 873 aa, Rat
MESLLGFLALLLLWGAVAEGPAKKVLTLEGDLVLGGLFPVHQKGGPAEECGPVNEHRGIQRLEAMLFALDRINRDPHLLPGVRLGAHILDSCSKDTHALEQALDFVRASLSRGADGSRHICPDGSYATHSDAPTAVTGVIGGSYSDVSIQVANLLRLFQIPQISYASTSAKLSDKSRYDYFARTVPPDFFQAKAMAEILRFFNWTYVSTVASEGDYGETGIEAFELEARARNICVATSEKVGRAMSRAAFEGVVRALLQKPSARVAVLFTRSEDARELLAATQRLNASFTWVASDGWGALESVVAGSERAAEGAITIELASYPISDFASYFQSLDPWNNSRNPWFREFWEERFHCSFRQRDCAAHSLRAVPFEQESKIMFVVNAVYAMAHALHNMHRALCPNTTHLCDAMRPVNGRRLYKDFVLNVKFDAPFRPADTDDEVRFDRFGDGIGRYNIFTYLRAGSGRYRYQKVGYWAEGLTLDTSFIPWASPSAGPLPASRCSEPCLQNEVKSVQPGEVCCWLCIPCQPYEYRLDEFTCADCGLGYWPNASLTGCFELPQEYIRWGDAWAVGPVTIACLGALATLFVLGVFVRHNATPVVKASGRELCYILLGGVFLCYCMTFVFIAKPSTAVCTLRRLGLGTAFSVCYSALLTKTNRIARIFGGAREGAQRPRFISPASQVAICLALILGQLLIVAAWLVVEAPGTGKETAPERREVVTLRCNHRDASMLGSLAYDVLLIALCTLYAFKTRKCPENFNEAKFIGFTMYTTCIIWLAFLPIFYVTSSDYRVQTTTMCVSVSLSGSVVLGCLFAPKLHIILFQPQKNVVSHRAPTSRFGSAAPRASANLGQGSGSQFVPTVCNGREVVDSTTSSLT

SEQ ID NO: 23 - protein, 873 aa, Rat
MESLLGFLALLLLWGAVAEGPAKKVLTLEGDLVLGGLFPVHQKGGPAEECGPVNEHRGIQRLEAMLFALDRINRDPHLLPGVRLGAHILDSCSKDTHALEQALDFVRASLSRGADGSRHICPDGSYATHSDAPTAVTGVIGGSYSDVSIQVANLLRLFQIPQISYASTSAKLSDKSRYDYFARTVPPDFFQAKAMAEILRFFNWTYVSTVASEGDYGETGIEAFELEARARNICVATSEKVGRAMSRAAFEGVVRALLQKPSARVAVLFTRSEDARELLAATQRLNASFTWVASDGWGALESVVAGSERAAEGAITIELASYPISDFASYFQSLDPWNNSRNPWFREFWEERFHCSFRQRDCAAHSLRAVPFEQESKIMFVVNAVYAMAHALHNMHRALCPNTTHLCDAMRPVNGRRLYKDFVLNVKFDAPFRPADTDDEVRFDRFGDGIGRYNIFTYLRAGSGRYRYQKVGYWAEGLTLDTSFIPWASPSAGPLPASRCSEPCLQNEVKSVQPGEVCCWLCIPCQPYEYRLDEFTCADCGLGYWPNASLTGCFELPQEYIRWGDAWAVGPVTIACLGALATLFVLGVFVRHNATPVVKASGRELCYILLGGVFLCYCMTFVFIAKPSTAVCTLRRLGLGTAFSVCYSALLTKTNRIARIFGGAREGAQRPRFISPASQVAICLALISVQLLIVAAWLVVEAPGTGKETAPERREVVTLRCNHRDASMLGSLAYNVLLIALCTLYAFKTRKCPENFNEAKFIGFTMYTTCIIWLAFLPIFYVTSSDYRVQTTTMCVSVSLSGSVVLGCLFAPKLHIILFQPQKNVVSHRAPTSRFGSAAPRASANLGQGSGSQFVPTVCNGREVVDSTTSSLT

SEQ ID NO: 24 - protein, 873 aa, Rat
MESLLGFLALLLLWGAVAEGPAKKVLTLEGDLVLGGLFPVHQKGGPAEECGPVNEHRGIQRLEAMLFALDRINRDPHLLPGVRLGAHILDSCSKDTHALEQALDFVRASLSRGADGSRHICPDGSYATHSDAPTAVTGVIGGSYSDVSIQVANLLRLFQIPQISYASTSAKLSDKSRYDYFARTVPPDFFQAKAMAEILRFFNWTYVSTVASEGDYGETGIEAFELEARARNICVATSEKVGRAMSRAAFEGVVRALLQKPSARVAVLFTRSEDARELLAATQRLNASFTWVASDGWGALESVVAGSERAAEGAITIELASYPISDFASYFQSLDPWNNSRNPWFREFWEERFHCSFRQRDCAAHSLRAVPFEQESKIMFVVNAVYAMAHALHNMHRALCPNTTHLCDAMRPVNGRRLYKDFVLNVKFDAPFRPADTDDEVRFDRFGDGIGRYNIFTYLRAGSGRYRYQKVGYWAEGLTLDTSFIPWASPSAGPLPASRCSEPCLQNEVKSVQPGEVCCWLCIPCQPYEYRLDEFTCADCGLGYWPNASLTGCFELPQEYIRWGDAWAVGPVTIACLGALATLFVLGVFVRHNATPVVKASGRELCYILLGGVFLCYCMTFVFIAKPSTAVCTLRRLGLGTAFSVCYSALLTKTNRIARIFGGAREGAQRPRFISPASQVAICLALILVQLLIVAAWLVVEAPGTGKETAPERREVVTLRCNHRDASMLGSLTYDVLLIALCTLYAFKTRKCPENFNEAKFIGFTMYTTCIIWLAFLPIFYVTSSDYRVQTTTMCVSVSLSGSVVLGCLFAPKLHIILFQPQKNVVSHRAPTSRFGSAAPRASANLGQGSGSQFVPTVCNGREVVDSTTSSLT

SEQ ID NO: 25 - DNA, 2619 bp, Rat
atggaatcactgcttgggtttctggcactgctgctgctgtggggtgccgtggccgagggcccggccaagaaggtgctgaccctggagggggacctggtgctgggtgggctgttcccagtgcaccagaagggtggcccagccgaggagtgtggacctgttaatgagcaccgaggcatacagcgcctagaggctatgctttttgcactggaccgcatcaaccgcgacccccacctgctgcctggtgtgcgcttgggtgcgcacatcctcgacagctgctccaaggatacacacgccctggagcaggcgctggactttgtgcgtgcctcactcagtcgtggcgctgacggctcacgccacatctgtcctgatggctcctatgccacccacagtgatgctcctacagctgtcaccggtgtcattggtggctcctacagtgatgtctccatccaggtggccaatctcctgcggctgttccagatcccacagatcagctatgcctccaccagtgccaagctgagtgacaagtcccgttacgattactttgctcgcactgtgcccccagacttcttccaagccaaggccatggctgagattctccgctttttcaactggacatatgtgtctacggtggcatctgagggtgactatggtgagacaggcattgaggctttcgagctcgaggctcgggcacgcaacatctgcgtggccacttctgagaaggtgggccgtgccatgagccgcgctgccttcgagggcgtggtgcgagccctgttgcagaaacccagtgcccgtgtggctgtgctcttcacccggtccgaggatgcccgtgagctgcttgcagccacccagcgcctcaacgccagcttcacatgggtggccagcgacggctggggggccctggagagcgtggtggcaggcagtgaaagggctgctgagggcgccatcaccattgaactggcctcctaccccatcagtgactttgcttcctacttccagagcttggatccctggaacaacagcagaaacccttggttccgtgagttctgggaggagaggttccattgcagcttccggcagcgagactgtgccgcccactctctgcgggccgtgccctttgaacaggagtcaaagatcatgtttgtggttaatgccgtctatgccatggcccacgctctacacaacatgcaccgtgccctctgtcccaacaccacccacctctgcgatgctatgaggcctgtcaatgggcgccgcctctacaaagacttcgtgctcaatgtcaagtttgacgccccctttcgcccagcagatactgacgatgaggtccgcttcgaccgctttggtgacggtattggccgctacaacatcttcacctatctgcgggcaggcagtgggcgctatcgctaccagaaggtaggctactgggcagaaggtctgactctggacactagcttcattccatgggcctccccatcagccggacctcttcctgcctctcgctgtagcgagccctgccttcagaacgaggtgaagagcgtgcagccgggcgaggtctgctgttggctctgcattccctgtcagccctatgagtacaggctggatgagttcacctgcgctgactgtggcctgggctactggcctaatgccagtctgactggctgctttgagctgccccaggagtacatccgctggggtgatgcctgggcggtgggacctgtcaccatcgcctgcctgggtgccctggcgacactctttgtgttgggtgtctttgtgaggcataatgccacacccgtggtcaaggcttccggtcgggagctttgctacattctgctgggcggtgtcttcctttgctattgtatgaccttcgtcttcattgctaagccttccacggccgtctgtaccttgaggcgcctcggtttgggtaccgccttctctgtctgctactcagccctcctcaccaagaccaatcgcattgctcgcatatttggcggggcccgggagggtgcccagcggccacgcttcatcagtcccgcctcacaggtggccatctgcttggcacttatctcgggccagctgctcattgtcgctgcctggctggtggtggaggcacctggcacaggcaaggagacagcccctgaacggcgggaagtggtgacattgcgctgtaaccaccgtgacgcgagcatgctcggctctctggcctacgatgtgctcctcatcgcgctctgcacgctctatgccttcaagacccgcaagtgcccggagaacttcaacgaagccaagttcatcggcttcaccatgtacaccacctgcatcatctggctggctttccttcctatcttctatgtcacctccagtgattatcgggtgcagaccaccacgatgtgcgtgtccgtcagcctcagtggctctgtggtgcttggctgcctctttgcacccaagttgcacatcatccttttccagccacagaagaatgtggtgagccaccgggcacctaccagccgctttggcagcgctgcccccagggccagcgccaaccttggtcaagggtctggatcccagtttgttcccactgtttgcaacggccgtgaggtggtggactcaacaacgtcgtcgctttga

SEQ ID NO: 26 - DNA, 2619 bp, Rat
atggaatcactgcttgggtttctggcactgctgctgctgtggggtgccgtggccgagggcccggccaagaaggtgctgaccctggagggggacctggtgctgggtgggctgttcccagtgcaccagaagggtggcccagccgaggagtgtggacctgttaatgagcaccgaggcatacagcgcctagaggctatgctttttgcactggaccgcatcaaccgcgacccccacctgctgcctggtgtgcgcttgggtgcgcacatcctcgacagctgctccaaggatacacacgccctggagcaggcgctggactttgtgcgtgcctcactcagtcgtggcgctgacggctcacgccacatctgtcctgatggctcctatgccacccacagtgatgctcctacagctgtcaccggtgtcattggtggctcctacagtgatgtctccatccaggtggccaatctcctgcggctgttccagatcccacagatcagctatgcctccaccagtgccaagctgagtgacaagtcccgttacgattactttgctcgcactgtgcccccagacttcttccaagccaaggccatggctgagattctccgctttttcaactggacatatgtgtctacggtggcatctgagggtgactatggtgagacaggcattgaggctttcgagctcgaggctcgggcacgcaacatctgcgtggccacttctgagaaggtgggccgtgccatgagccgcgctgccttcgagggcgtggtgcgagccctgttgcagaaacccagtgcccgtgtggctgtgctcttcacccggtccgaggatgcccgtgagctgcttgcagccacccagcgcctcaacgccagcttcacatgggtggccagcgacggctggggggccctggagagcgtggtggcaggcagtgaaagggctgctgagggcgccatcaccattgaactggcctcctaccccatcagtgactttgcttcctacttccagagcttggatccctggaacaacagcagaaacccttggttccgtgagttctgggaggagaggttccattgcagcttccggcagcgagactgtgccgcccactctctgcgggccgtgccctttgaacaggagtcaaagatcatgtttgtggttaatgccgtctatgccatggcccacgctctacacaacatgcaccgtgccctctgtcccaacaccacccacctctgcgatgctatgaggcctgtcaatgggcgccgcctctacaaagacttcgtgctcaatgtcaagtttgacgccccctttcgcccagcagatactgacgatgaggtccgcttcgaccgctttggtgacggtattggccgctacaacatcttcacctatctgcgggcaggcagtgggcgctatcgctaccagaaggtaggctactgggcagaaggtctgactctggacactagcttcattccatgggcctccccatcagccggacctcttcctgcctctcgctgtagcgagccctgccttcagaacgaggtgaagagcgtgcagccgggcgaggtctgctgttggctctgcattccctgtcagccctatgagtacaggctggatgagttcacctgcgctgactgtggcctgggctactggcctaatgccagtctgactggctgctttgagctgccccaggagtacatccgctggggtgatgcctgggcggtgggacctgtcaccatcgcctgcctgggtgccctggcgacactctttgtgttgggtgtctttgtgaggcataatgccacacccgtggtcaaggcttccggtcgggagctttgctacattctgctgggcggtgtcttcctttgctattgtatgaccttcgtcttcattgctaagccttccacggccgtctgtaccttgaggcgcctcggtttgggtaccgccttctctgtctgctactcagccctcctcaccaagaccaatcgcattgctcgcatatttggcggggcccgggagggtgcccagcggccacgcttcatcagtcccgcctcacaggtggccatctgcttggcacttatctcggtccagctgctcattgtcgctgcctggctggtggtggaggcacctggcacaggcaaggagacagcccctgaacggcgggaagtggtgacattgcgctgtaaccaccgtgacgcgagcatgctcggctctctggcctacgatgtgctcctcatcgcgctctgcacgctctatgccttcaagacccgcaagtgcccggagaacttcaacgaagccaagttcatcggcttcaccatgtacaccacctgcatcatctggctggctttccttcctatcttctatgtcacctccagtgattatcgggtgcagaccaccacgatgtgcgtgtccgtcagcctcagtggctctgtggtgcttggctgcctctttgcacccaagttgcacatcatccttttccagccacagaagaatgtggtgagccaccgggcacctaccagccgctttggcagcgctgcccccagggccagcgccaaccttggtcaagggtctggatcccagtttgttcccactgtttgcaacggccgtgaggtggtggactcaacaacgtcgtcgctttga

SEQ ID NO: 27 - DNA, 2619 bp, Rat
atggaatcactgcttgggtttctggcactgctgctgctgtggggtgccgtggccgagggcccggccaagaaggtgctgaccctggagggggacctggtgctgggtgggctgttcccagtgcaccagaagggtggcccagccgaggagtgtggacctgttaatgagcaccgaggcatacagcgcctagaggctatgctttttgcactggaccgcatcaaccgcgacccccacctgctgcctggtgtgcgcttgggtgcgcacatcctcgacagctgctccaaggatacacacgccctggagcaggcgctggactttgtgcgtgcctcactcagtcgtggcgctgacggctcacgccacatctgtcctgatggctcctatgccacccacagtgatgctcctacagctgtcaccggtgtcattggtggctcctacagtgatgtctccatccaggtggccaatctcctgcggctgttccagatcccacagatcagctatgcctccaccagtgccaagctgagtgacaagtcccgttacgattactttgctcgcactgtgcccccagacttcttccaagccaaggccatggctgagattctccgctttttcaactggacatatgtgtctacggtggcatctgagggtgactatggtgagacaggcattgaggctttcgagctcgaggctcgggcacgcaacatctgcgtggccacttctgagaaggtgggccgtgccatgagccgcgctgccttcgagggcgtggtgcgagccctgttgcagaaacccagtgcccgtgtggctgtgctcttcacccggtccgaggatgcccgtgagctgcttgcagccacccagcgcctcaacgccagcttcacatgggtggccagcgacggctggggggccctggagagcgtggtggcaggcagtgaaagggctgctgagggcgccatcaccattgaactggcctcctaccccatcagtgactttgcttcctacttccagagcttggatccctggaacaacagcagaaacccttggttccgtgagttctgggaggagaggttccattgcagcttccggcagcgagactgtgccgcccactctctgcgggccgtgccctttgaacaggagtcaaagatcatgtttgtggttaatgccgtctatgccatggcccacgctctacacaacatgcaccgtgccctctgtcccaacaccacccacctctgcgatgctatgaggcctgtcaatgggcgccgcctctacaaagacttcgtgctcaatgtcaagtttgacgccccctttcgcccagcagatactgacgatgaggtccgcttcgaccgctttggtgacggtattggccgctacaacatcttcacctatctgcgggcaggcagtgggcgctatcgctaccagaaggtaggctactgggcagaaggtctgactctggacactagcttcattccatgggcctccccatcagccggacctcttcctgcctctcgctgtagcgagccctgccttcagaacgaggtgaagagcgtgcagccgggcgaggtctgctgttggctctgcattccctgtcagccctatgagtacaggctggatgagttcacctgcgctgactgtggcctgggctactggcctaatgccagtctgactggctgctttgagctgccccaggagtacatccgctggggtgatgcctgggcggtgggacctgtcaccatcgcctgcctgggtgccctggcgacactctttgtgttgggtgtctttgtgaggcataatgccacacccgtggtcaaggcttccggtcgggagctttgctacattctgctgggcggtgtcttcctttgctattgtatgaccttcgtcttcattgctaagccttccacggccgtctgtaccttgaggcgcctcggtttgggtaccgccttctctgtctgctactcagccctcctcaccaagaccaatcgcattgctcgcatatttggcggggcccgggagggtgcccagcggccacgcttcatcagtcccgcctcacaggtggccatctgcttggcacttatcttggtccagctgctcattgtcgctgcctggctggtggtggaggcacctggcacaggcaaggagacagcccctgaacggcgggaagtggtgacattgcgctgtaaccaccgtgacgcgagcatgctcggctctctggcctacgatgtgctcctcatcgcgctctgcacgctctatgccttcaagacccgcaagtgcccggagaacttcaacgaagccaagttcatcggcttcaccatgtacaccacctgcatcatctggctggctttccttcctatcttctatgtcacctccagtgattatcgggtgcagaccaccacgatgtgcgtgtccgtcagcctcagtggctctgtggtgcttggctgcctctttgcacccaagttgcacatcatccttttccagccacagaagaatgtggtgagccaccgggcacctaccagccgctttggcagcgctgcccccagggccagcgccaaccttggtcaagggtctggatcccagtttgttcccactgtttgcaacggccgtgaggtggtggactcaacaacgtcgtcgctttga

SEQ ID NO: 28 - DNA, 2619 bp, Rat
atggaatcactgcttgggtttctggcactgctgctgctgtggggtgccgtggccgagggcccggccaagaaggtgctgaccctggagggggacctggtgctgggtgggctgttcccagtgcaccagaagggtggcccagccgaggagtgtggacctgttaatgagcaccgaggcatacagcgcctagaggctatgctttttgcactggaccgcatcaaccgcgacccccacctgctgcctggtgtgcgcttgggtgcgcacatcctcgacagctgctccaaggatacacacgccctggagcaggcgctggactttgtgcgtgcctcactcagtcgtggcgctgacggctcacgccacatctgtcctgatggctcctatgccacccacagtgatgctcctacagctgtcaccggtgtcattggtggctcctacagtgatgtctccatccaggtggccaatctcctgcggctgttccagatcccacagatcagctatgcctccaccagtgccaagctgagtgacaagtcccgttacgattactttgctcgcactgtgcccccagacttcttccaagccaaggccatggctgagattctccgctttttcaactggacatatgtgtctacggtggcatctgagggtgactatggtgagacaggcattgaggctttcgagctcgaggctcgggcacgcaacatctgcgtggccacttctgagaaggtgggccgtgccatgagccgcgctgccttcgagggcgtggtgcgagccctgttgcagaaacccagtgcccgtgtggctgtgctcttcacccggtccgaggatgcccgtgagctgcttgcagccacccagcgcctcaacgccagcttcacatgggtggccagcgacggctggggggccctggagagcgtggtggcaggcagtgaaagggctgctgagggcgccatcaccattgaactggcctcctaccccatcagtgactttgcttcctacttccagagcttggatccctggaacaacagcagaaacccttggttccgtgagttctgggaggagaggttccattgcagcttccggcagcgagactgtgccgcccactctctgcgggccgtgccctttgaacaggagtcaaagatcatgtttgtggttaatgccgtctatgccatggcccacgctctacacaacatgcaccgtgccctctgtcccaacaccacccacctctgcgatgctatgaggcctgtcaatgggcgccgcctctacaaagacttcgtgctcaatgtcaagtttgacgccccctttcgcccagcagatactgacgatgaggtccgcttcgaccgctttggtgacggtattggccgctacaacatcttcacctatctgcgggcaggcagtgggcgctatcgctaccagaaggtaggctactgggcagaaggtctgactctggacactagcttcattccatgggcctccccatcagccggacctcttcctgcctctcgctgtagcgagccctgccttcagaacgaggtgaagagcgtgcagccgggcgaggtctgctgttggctctgcattccctgtcagccctatgagtacaggctggatgagttcacctgcgctgactgtggcctgggctactggcctaatgccagtctgactggctgctttgagctgccccaggagtacatccgctggggtgatgcctgggcggtgggacctgtcaccatcgcctgcctgggtgccctggcgacactctttgtgttgggtgtctttgtgaggcataatgccacacccgtggtcaaggcttccggtcgggagctttgctacattctgctgggcggtgtcttcctttgctattgtatgaccttcgtcttcattgctaagccttccacggccgtctgtaccttgaggcgcctcggtttgggtaccgccttctctgtctgctactcagccctcctcaccaagaccaatcgcattgctcgcatatttggcggggcccgggagggtgcccagcggccacgcttcatcagtcccgcctcacaggtggccatctgcttggcacttatcttgggccagctgctcattgtcgctgcctggctggtggtggaggcacctggcacaggcaaggagacagcccctgaacggcgggaagtggtgacattgcgctgtaaccaccgtgacgcgagcatgctcggctctctggcctacaatgtgctcctcatcgcgctctgcacgctctatgccttcaagacccgcaagtgcccggagaacttcaacgaagccaagttcatcggcttcaccatgtacaccacctgcatcatctggctggctttccttcctatcttctatgtcacctccagtgattatcgggtgcagaccaccacgatgtgcgtgtccgtcagcctcagtggctctgtggtgcttggctgcctctttgcacccaagttgcacatcatccttttccagccacagaagaatgtggtgagccaccgggcacctaccagccgctttggcagcgctgcccccagggccagcgccaaccttggtcaagggtctggatcccagtttgttcccactgtttgcaacggccgtgaggtggtggactcaacaacgtcgtcgctttga

SEQ ID NO: 29 - DNA, 2619 bp, Rat
atggaatcactgcttgggtttctggcactgctgctgctgtggggtgccgtggccgagggcccggccaagaaggtgctgaccctggagggggacctggtgctgggtgggctgttcccagtgcaccagaagggtggcccagccgaggagtgtggacctgttaatgagcaccgaggcatacagcgcctagaggctatgctttttgcactggaccgcatcaaccgcgacccccacctgctgcctggtgtgcgcttgggtgcgcacatcctcgacagctgctccaaggatacacacgccctggagcaggcgctggactttgtgcgtgcctcactcagtcgtggcgctgacggctcacgccacatctgtcctgatggctcctatgccacccacagtgatgctcctacagctgtcaccggtgtcattggtggctcctacagtgatgtctccatccaggtggccaatctcctgcggctgttccagatcccacagatcagctatgcctccaccagtgccaagctgagtgacaagtcccgttacgattactttgctcgcactgtgcccccagacttcttccaagccaaggccatggctgagattctccgctttttcaactggacatatgtgtctacggtggcatctgagggtgactatggtgagacaggcattgaggctttcgagctcgaggctcgggcacgcaacatctgcgtggccacttctgagaaggtgggccgtgccatgagccgcgctgccttcgagggcgtggtgcgagccctgttgcagaaacccagtgcccgtgtggctgtgctcttcacccggtccgaggatgcccgtgagctgcttgcagccacccagcgcctcaacgccagcttcacatgggtggccagcgacggctggggggccctggagagcgtggtggcaggcagtgaaagggctgctgagggcgccatcaccattgaactggcctcctaccccatcagtgactttgcttcctacttccagagcttggatccctggaacaacagcagaaacccttggttccgtgagttctgggaggagaggttccattgcagcttccggcagcgagactgtgccgcccactctctgcgggccgtgccctttgaacaggagtcaaagatcatgtttgtggttaatgccgtctatgccatggcccacgctctacacaacatgcaccgtgccctctgtcccaacaccacccacctctgcgatgctatgaggcctgtcaatgggcgccgcctctacaaagacttcgtgctcaatgtcaagtttgacgccccctttcgcccagcagatactgacgatgaggtccgcttcgaccgctttggtgacggtattggccgctacaacatcttcacctatctgcgggcaggcagtgggcgctatcgctaccagaaggtaggctactgggcagaaggtctgactctggacactagcttcattccatgggcctccccatcagccggacctcttcctgcctctcgctgtagcgagccctgccttcagaacgaggtgaagagcgtgcagccgggcgaggtctgctgttggctctgcattccctgtcagccctatgagtacaggctggatgagttcacctgcgctgactgtggcctgggctactggcctaatgccagtctgactggctgctttgagctgccccaggagtacatccgctggggtgatgcctgggcggtgggacctgtcaccatcgcctgcctgggtgccctggcgacactctttgtgttgggtgtctttgtgaggcataatgccacacccgtggtcaaggcttccggtcgggagctttgctacattctgctgggcggtgtcttcctttgctattgtatgaccttcgtcttcattgctaagccttccacggccgtctgtaccttgaggcgcctcggtttgggtaccgccttctctgtctgctactcagccctcctcaccaagaccaatcgcattgctcgcatatttggcggggcccgggagggtgcccagcggccacgcttcatcagtcccgcctcacaggtggccatctgcttggcacttatcttggtccagctgctcattgtcgctgcctggctggtggtggaggcacctggcacaggcaaggagacagcccctgaacggcgggaagtggtgacattgcgctgtaaccaccgtgacgcgagcatgctcggctctctggcctacaatgtgctcctcatcgcgctctgcacgctctatgccttcaagacccgcaagtgcccggagaacttcaacgaagccaagttcatcggcttcaccatgtacaccacctgcatcatctggctggctttccttcctatcttctatgtcacctccagtgattatcgggtgcagaccaccacgatgtgcgtgtccgtcagcctcagtggctctgtggtgcttggctgcctctttgcacccaagttgcacatcatccttttccagccacagaagaatgtggtgagccaccgggcacctaccagccgctttggcagcgctgcccccagggccagcgccaaccttggtcaagggtctggatcccagtttgttcccactgtttgcaacggccgtgaggtggtggactcaacaacgtcgtcgctttga

SEQ ID NO: 30 - DNA, 2619 bp, Rat
atggaatcactgcttgggtttctggcactgctgctgctgtggggtgccgtggccgagggcccggccaagaaggtgctgaccctggagggggacctggtgctgggtgggctgttcccagtgcaccagaagggtggcccagccgaggagtgtggacctgttaatgagcaccgaggcatacagcgcctagaggctatgctttttgcactggaccgcatcaaccgcgacccccacctgctgcctggtgtgcgcttgggtgcgcacatcctcgacagctgctccaaggatacacacgccctggagcaggcgctggactttgtgcgtgcctcactcagtcgtggcgctgacggctcacgccacatctgtcctgatggctcctatgccacccacagtgatgctcctacagctgtcaccggtgtcattggtggctcctacagtgatgtctccatccaggtggccaatctcctgcggctgttccagatcccacagatcagctatgcctccaccagtgccaagctgagtgacaagtcccgttacgattactttgctcgcactgtgcccccagacttcttccaagccaaggccatggctgagattctccgctttttcaactggacatatgtgtctacggtggcatctgagggtgactatggtgagacaggcattgaggctttcgagctcgaggctcgggcacgcaacatctgcgtggccacttctgagaaggtgggccgtgccatgagccgcgctgccttcgagggcgtggtgcgagccctgttgcagaaacccagtgcccgtgtggctgtgctcttcacccggtccgaggatgcccgtgagctgcttgcagccacccagcgcctcaacgccagcttcacatgggtggccagcgacggctggggggccctggagagcgtggtggcaggcagtgaaagggctgctgagggcgccatcaccattgaactggcctcctaccccatcagtgactttgcttcctacttccagagcttggatccctggaacaacagcagaaacccttggttccgtgagttctgggaggagaggttccattgcagcttccggcagcgagactgtgccgcccactctctgcgggccgtgccctttgaacaggagtcaaagatcatgtttgtggttaatgccgtctatgccatggcccacgctctacacaacatgcaccgtgccctctgtcccaacaccacccacctctgcgatgctatgaggcctgtcaatgggcgccgcctctacaaagacttcgtgctcaatgtcaagtttgacgccccctttcgcccagcagatactgacgatgaggtccgcttcgaccgctttggtgacggtattggccgctacaacatcttcacctatctgcgggcaggcagtgggcgctatcgctaccagaaggtaggctactgggcagaaggtctgactctggacactagcttcattccatgggcctccccatcagccggacctcttcctgcctctcgctgtagcgagccctgccttcagaacgaggtgaagagcgtgcagccgggcgaggtctgctgttggctctgcattccctgtcagccctatgagtacaggctggatgagttcacctgcgctgactgtggcctgggctactggcctaatgccagtctgactggctgctttgagctgccccaggagtacatccgctggggtgatgcctgggcggtgggacctgtcaccatcgcctgcctgggtgccctggcgacactctttgtgttgggtgtctttgtgaggcataatgccacacccgtggtcaaggcttccggtcgggagctttgctacattctgctgggcggtgtcttcctttgctattgtatgaccttcgtcttcattgctaagccttccacggccgtctgtaccttgaggcgcctcggtttgggtaccgccttctctgtctgctactcagccctcctcaccaagaccaatcgcattgctcgcatatttggcggggcccgggagggtgcccagcggccacgcttcatcagtcccgcctcacaggtggccatctgcttggcacttatcttgggccagctgctcattgtcgctgcctggctggtggtggaggcacctggcacaggcaaggagacagcccctgaacggcgggaagtggtgacattgcgctgtaaccaccgtgacgcgagcatgctcggctctctggcctacgatgtgctcctcatcgcgctctgcacgctctatgccttcaagacccgcaagtgcccggagaacttcaacgaagccaagttcatcggcttcaccatgtacaccacctgcatcatctggctggctttccttcctatcttctatgtcacctccagtgattatcgggtgcagaccaccacgatgtgcgtgtccgtcagcctcagtggctctgtggtgcttggctgcctctttgcacccaagttgcacatcatccttttccagccacagaagaatgtggtgagccaccgggcacctaccagccgctttggcagcgctgcccccagggccagcgccaaccttggtcaagggtctggatcccagtttgttcccactgtttgcaacggccgtgaggtggtggactcaacaacgtcgtcgctttga

SEQ ID NO: 31 - DNA, 2619 bp, Rat
atggaatcactgcttgggtttctggcactgctgctgctgtggggtgccgtggccgagggcccggccaagaaggtgctgaccctggagggggacctggtgctgggtgggctgttcccagtgcaccagaagggtggcccagccgaggagtgtggacctgttaatgagcaccgaggcatacagcgcctagaggctatgctttttgcactggaccgcatcaaccgcgacccccacctgctgcctggtgtgcgcttgggtgcgcacatcctcgacagctgctccaaggatacacacgccctggagcaggcgctggactttgtgcgtgcctcactcagtcgtggcgctgacggctcacgccacatctgtcctgatggctcctatgccacccacagtgatgctcctacagctgtcaccggtgtcattggtggctcctacagtgatgtctccatccaggtggccaatctcctgcggctgttccagatcccacagatcagctatgcctccaccagtgccaagctgagtgacaagtcccgttacgattactttgctcgcactgtgcccccagacttcttccaagccaaggccatggctgagattctccgctttttcaactggacatatgtgtctacggtggcatctgagggtgactatggtgagacaggcattgaggctttcgagctcgaggctcgggcacgcaacatctgcgtggccacttctgagaaggtgggccgtgccatgagccgcgctgccttcgagggcgtggtgcgagccctgttgcagaaacccagtgcccgtgtggctgtgctcttcacccggtccgaggatgcccgtgagctgcttgcagccacccagcgcctcaacgccagcttcacatgggtggccagcgacggctggggggccctggagagcgtggtggcaggcagtgaaagggctgctgagggcgccatcaccattgaactggcctcctaccccatcagtgactttgcttcctacttccagagcttggatccctggaacaacagcagaaacccttggttccgtgagttctgggaggagaggttccattgcagcttccggcagcgagactgtgccgcccactctctgcgggccgtgccctttgaacaggagtcaaagatcatgtttgtggttaatgccgtctatgccatggcccacgctctacacaacatgcaccgtgccctctgtcccaacaccacccacctctgcgatgctatgaggcctgtcaatgggcgccgcctctacaaagacttcgtgctcaatgtcaagtttgacgccccctttcgcccagcagatactgacgatgaggtccgcttcgaccgctttggtgacggtattggccgctacaacatcttcacctatctgcgggcaggcagtgggcgctatcgctaccagaaggtaggctactgggcagaaggtctgactctggacactagcttcattccatgggcctccccatcagccggacctcttcctgcctctcgctgtagcgagccctgccttcagaacgaggtgaagagcgtgcagccgggcgaggtctgctgttggctctgcattccctgtcagccctatgagtacaggctggatgagttcacctgcgctgactgtggcctgggctactggcctaatgccagtctgactggctgctttgagctgccccaggagtacatccgctggggtgatgcctgggcggtgggacctgtcaccatcgcctgcctgggtgccctggcgacactctttgtgttgggtgtctttgtgaggcataatgccacacccgtggtcaaggcttccggtcgggagctttgctacattctgctgggcggtgtcttcctttgctattgtatgaccttcgtcttcattgctaagccttccacggccgtctgtaccttgaggcgcctcggtttgggtaccgccttctctgtctgctactcagccctcctcaccaagaccaatcgcattgctcgcatatttggcggggcccgggagggtgcccagcggccacgcttcatcagtcccgcctcacaggtggccatctgcttggcacttatctcggtccagctgctcattgtcgctgcctggctggtggtggaggcacctggcacaggcaaggagacagcccctgaacggcgggaagtggtgacattgcgctgtaaccaccgtgacgcgagcatgctcggctctctggcctacaatgtgctcctcatcgcgctctgcacgctctatgccttcaagacccgcaagtgcccggagaacttcaacgaagccaagttcatcggcttcaccatgtacaccacctgcatcatctggctggctttccttcctatcttctatgtcacctccagtgattatcgggtgcagaccaccacgatgtgcgtgtccgtcagcctcagtggctctgtggtgcttggctgcctctttgcacccaagttgcacatcatccttttccagccacagaagaatgtggtgagccaccgggcacctaccagccgctttggcagcgctgcccccagggccagcgccaaccttggtcaagggtctggatcccagtttgttcccactgtttgcaacggccgtgaggtggtggactcaacaacgtcgtcgctttga

SEQ ID NO: 32 - DNA, 2619 bp, Rat
atggaatcactgcttgggtttctggcactgctgctgctgtggggtgccgtggccgagggcccggccaagaaggtgctgaccctggagggggacctggtgctgggtgggctgttcccagtgcaccagaagggtggcccagccgaggagtgtggacctgttaatgagcaccgaggcatacagcgcctagaggctatgctttttgcactggaccgcatcaaccgcgacccccacctgctgcctggtgtgcgcttgggtgcgcacatcctcgacagctgctccaaggatacacacgccctggagcaggcgctggactttgtgcgtgcctcactcagtcgtggcgctgacggctcacgccacatctgtcctgatggctcctatgccacccacagtgatgctcctacagctgtcaccggtgtcattggtggctcctacagtgatgtctccatccaggtggccaatctcctgcggctgttccagatcccacagatcagctatgcctccaccagtgccaagctgagtgacaagtcccgttacgattactttgctcgcactgtgcccccagacttcttccaagccaaggccatggctgagattctccgctttttcaactggacatatgtgtctacggtggcatctgagggtgactatggtgagacaggcattgaggctttcgagctcgaggctcgggcacgcaacatctgcgtggccacttctgagaaggtgggccgtgccatgagccgcgctgccttcgagggcgtggtgcgagccctgttgcagaaacccagtgcccgtgtggctgtgctcttcacccggtccgaggatgcccgtgagctgcttgcagccacccagcgcctcaacgccagcttcacatgggtggccagcgacggctggggggccctggagagcgtggtggcaggcagtgaaagggctgctgagggcgccatcaccattgaactggcctcctaccccatcagtgactttgcttcctacttccagagcttggatccctggaacaacagcagaaacccttggttccgtgagttctgggaggagaggttccattgcagcttccggcagcgagactgtgccgcccactctctgcgggccgtgccctttgaacaggagtcaaagatcatgtttgtggttaatgccgtctatgccatggcccacgctctacacaacatgcaccgtgccctctgtcccaacaccacccacctctgcgatgctatgaggcctgtcaatgggcgccgcctctacaaagacttcgtgctcaatgtcaagtttgacgccccctttcgcccagcagatactgacgatgaggtccgcttcgaccgctttggtgacggtattggccgctacaacatcttcacctatctgcgggcaggcagtgggcgctatcgctaccagaaggtaggctactgggcagaaggtctgactctggacactagcttcattccatgggcctccccatcagccggacctcttcctgcctctcgctgtagcgagccctgccttcagaacgaggtgaagagcgtgcagccgggcgaggtctgctgttggctctgcattccctgtcagccctatgagtacaggctggatgagttcacctgcgctgactgtggcctgggctactggcctaatgccagtctgactggctgctttgagctgccccaggagtacatccgctggggtgatgcctgggcggtgggacctgtcaccatcgcctgcctgggtgccctggcgacactctttgtgttgggtgtctttgtgaggcataatgccacacccgtggtcaaggcttccggtcgggagctttgctacattctgctgggcggtgtcttcctttgctattgtatgaccttcgtcttcattgctaagccttccacggccgtctgtaccttgaggcgcctcggtttgggtaccgccttctctgtctgctactcagccctcctcaccaagaccaatcgcattgctcgcatatttggcggggcccgggagggtgcccagcggccacgcttcatcagtcccgcctcacaggtggccatctgcttggcacttatcttggtccagctgctcattgtcgctgcctggctggtggtggaggcacctggcacaggcaaggagacagcccctgaacggcgggaagtggtgacattgcgctgtaaccaccgtgacgcgagcatgctcggctctctgacctacgatgtgctcctcatcgcgctctgcacgctctatgccttcaagacccgcaagtgcccggagaacttcaacgaagccaagttcatcggcttcaccatgtacaccacctgcatcatctggctggctttccttcctatcttctatgtcacctccagtgattatcgggtgcagaccaccacgatgtgcgtgtccgtcagcctcagtggctctgtggtgcttggctgcctctttgcacccaagttgcacatcatccttttccagccacagaagaatgtggtgagccaccgggcacctaccagccgctttggcagcgctgcccccagggccagcgccaaccttggtcaagggtctggatcccagtttgttcccactgtttgcaacggccgtgaggtggtggactcaacaacgtcgtcgctttga

SEQ ID NO: 33 - protein, 880 aa, Human
MKMLTRLQVLTLALFSKGFLLSLGDHNFLRREIKIEGDLVLGGLFPINEKGTGTEECGRINEDRGIQRLEAMLFAIDEINKDDYLLPGVKLGVHILDTCSRDTYALEQSLEFVRASLTKVDEAEYMCPDGSYAIQENIPLLIAGVIGGSYSSVSIQVANLLRLFQIPQISYASTSAKLSDKSRYDYFARTVPPDFYQAKAMAEILRFFNWTYVSTVASEGDYGETGIEAFEQEARLRNICIATAEKVGRSNIRKSYDSVIRELLQKPNARVVVLFMRSDDSRELIAAASRANASFTWVASDGWGAQESIIKGSEHVAYGDITLELASQPVRQFGRYFQSLNPYNNHRNPWFRDFWEQKFQCSLQNKRNHRRVCEKHLAIDSSNYEQESKIMFVVNAVYAMAHALHKMQRTLCPNTTKLCDAMKILDGKKLYKDYLLKINFTAPFNPNKDADSIVKFDTFGDGMGRYNVFNFQNVGGKYSYLKVGHWAETLSLDVNSIHWSRNSVPTSQCSDPCAPNEMKNMQPGDVCCWICIPCEPYEYLADEFTCMDCGSGQWPTADLTGCYDLPEDYIRWEDAWAIGPVTIACLGFMCTCMVVTVFIKHNNTPLVKASGRELCYILLFGVGLSYCMTFFFIAKPSPVICALRRLGLGSSFAICYSALLTKTNCIARIFDGVKNGAQRPKFISPSSQVFICLGLILVQIVMVSVWLILEAPGTRRYTLAEKRETVILKCNVKDSSMLISLTYNVILVILCTVYAFKTRKCPENFNEAKFIGFTMYTTCIIWLAFLPIFYVTSSDYRVQTTTMCISVSLSGFVVLGCLFAPKVHIILFQPQKNVVTHRLHLNRFSVSGTGTTYSQSSASTYVPTVCNGREVLDSTTSSLT

SEQ ID NO: 34 - protein, 880 aa, Human
MKMLTRLQVLTLALFSKGFLLSLGDHNFLRREIKIEGDLVLGGLFPINEKGTGTEECGRINEDRGIQRLEAMLFAIDEINKDDYLLPGVKLGVHILDTCSRDTYALEQSLEFVRASLTKVDEAEYMCPDGSYAIQENIPLLIAGVIGGSYSSVSIQVANLLRLFQIPQISYASTSAKLSDKSRYDYFARTVPPDFYQAKAMAEILRFFNWTYVSTVASEGDYGETGIEAFEQEARLRNICIATAEKVGRSNIRKSYDSVIRELLQKPNARVVVLFMRSDDSRELIAAASRANASFTWVASDGWGAQESIIKGSEHVAYGDITLELASQPVRQFGRYFQSLNPYNNHRNPWFRDFWEQKFQCSLQNKRNHRRVCEKHLAIDSSNYEQESKIMFVVNAVYAMAHALHKMQRTLCPNTTKLCDAMKILDGKKLYKDYLLKINFTAPFNPNKDADSIVKFDTFGDGMGRYNVFNFQNVGGKYSYLKVGHWAETLSLDVNSIHWSRNSVPTSQCSDPCAPNEMKNMQPGDVCCWICIPCEPYEYLADEFTCMDCGSGQWPTADLTGCYDLPEDYIRWEDAWAIGPVTIACLGFMCTCMVVTVFIKHNNTPLVKASGRELCYILLFGVGLSYCMTFFFIAKPSPVICALRRLGLGSSFAICYSALLTKTNCIARIFDGVKNGAQRPKFISPSSQVFICLGLILGQIVMVSVWLILEAPGTRRYTLAEKRETVILKCNVKDSSMLISLTYNVILVILCTVYAFKTRKCPENFNEAKFIGFTMYTTCIIWLAFLPIFYVTSSDYRVQTTTMCISVSLSGFVVLGCLFAPKVHIILFQPQKNVVTHRLHLNRFSVSGTGTTYSQSSASTYVPTVCNGREVLDSTTSSLT

SEQ ID NO: 35 - protein, 880 aa, Human
MKMLTRLQVLTLALFSKGFLLSLGDHNFLRREIKIEGDLVLGGLFPINEKGTGTEECGRINEDRGIQRLEAMLFAIDEINKDDYLLPGVKLGVHILDTCSRDTYALEQSLEFVRASLTKVDEAEYMCPDGSYAIQENIPLLIAGVIGGSYSSVSIQVANLLRLFQIPQISYASTSAKLSDKSRYDYFARTVPPDFYQAKAMAEILRFFNWTYVSTVASEGDYGETGIEAFEQEARLRNICIATAEKVGRSNIRKSYDSVIRELLQKPNARVVVLFMRSDDSRELIAAASRANASFTWVASDGWGAQESIIKGSEHVAYGDITLELASQPVRQFGRYFQSLNPYNNHRNPWFRDFWEQKFQCSLQNKRNHRRVCEKHLAIDSSNYEQESKIMFVVNAVYAMAHALHKMQRTLCPNTTKLCDAMKILDGKKLYKDYLLKINFTAPFNPNKDADSIVKFDTFGDGMGRYNVFNFQNVGGKYSYLKVGHWAETLSLDVNSIHWSRNSVPTSQCSDPCAPNEMKNMQPGDVCCWICIPCEPYEYLADEFTCMDCGSGQWPTADLTGCYDLPEDYIRWEDAWAIGPVTIACLGFMCTCMVVTVFIKHNNTPLVKASGRELCYILLFGVGLSYCMTFFFIAKPSPVICALRRLGLGSSFAICYSALLTKTNCIARIFDGVKNGAQRPKFISPSSQVFICLGLISGQIVMVSVWLILEAPGTRRYTLAEKRETVILKCNVKDSSMLISLTYNVILVILCTVYAFKTRKCPENFNEAKFIGFTMYTTCIIWLAFLPIFYVTSSDYRVQTTTMCISVSLSGFVVLGCLFAPKVHIILFQPQKNVVTHRLHLNRFSVSGTGTTYSQSSASTYVPTVCNGREVLDSTTSSLT

SEQ ID NO: 36 - protein, 880 aa, Human
MKMLTRLQVLTLALFSKGFLLSLGDHNFLRREIKIEGDLVLGGLFPINEKGTGTEECGRINEDRGIQRLEAMLFAIDEINKDDYLLPGVKLGVHILDTCSRDTYALEQSLEFVRASLTKVDEAEYMCPDGSYAIQENIPLLIAGVIGGSYSSVSIQVANLLRLFQIPQISYASTSAKLSDKSRYDYFARTVPPDFYQAKAMAEILRFFNWTYVSTVASEGDYGETGIEAFEQEARLRNICIATAEKVGRSNIRKSYDSVIRELLQKPNARVVVLFMRSDDSRELIAAASRANASFTWVASDGWGAQESIIKGSEHVAYGDITLELASQPVRQFGRYFQSLNPYNNHRNPWFRDFWEQKFQCSLQNKRNHRRVCEKHLAIDSSNYEQESKIMFVVNAVYAMAHALHKMQRTLCPNTTKLCDAMKILDGKKLYKDYLLKINFTAPFNPNKDADSIVKFDTFGDGMGRYNVFNFQNVGGKYSYLKVGHWAETLSLDVNSIHWSRNSVPTSQCSDPCAPNEMKNMQPGDVCCWICIPCEPYEYLADEFTCMDCGSGQWPTADLTGCYDLPEDYIRWEDAWAIGPVTIACLGFMCTCMVVTVFIKHNNTPLVKASGRELCYILLFGVGLSYCMTFFFIAKPSPVICALRRLGLGSSFAICYSALLTKTNCIARIFDGVKNGAQRPKFISPSSQVFICLGLISVQIVMVSVWLILEAPGTRRYTLAEKRETVILKCNVKDSSMLISLTYDVILVILCTVYAFKTRKCPENFNEAKFIGFTMYTTCIIWLAFLPIFYVTSSDYRVQTTTMCISVSLSGFVVLGCLFAPKVHIILFQPQKNVVTHRLHLNRFSVSGTGTTYSQSSASTYVPTVCNGREVLDSTTSSLT

SEQ ID NO: 37 - protein, 880 aa, Human
MKMLTRLQVLTLALFSKGFLLSLGDHNFLRREIKIEGDLVLGGLFPINEKGTGTEECGRINEDRGIQRLEAMLFAIDEINKDDYLLPGVKLGVHILDTCSRDTYALEQSLEFVRASLTKVDEAEYMCPDGSYAIQENIPLLIAGVIGGSYSSVSIQVANLLRLFQIPQISYASTSAKLSDKSRYDYFARTVPPDFYQAKAMAEILRFFNWTYVSTVASEGDYGETGIEAFEQEARLRNICIATAEKVGRSNIRKSYDSVIRELLQKPNARVVVLFMRSDDSRELIAAASRANASFTWVASDGWGAQESIIKGSEHVAYGDITLELASQPVRQFGRYFQSLNPYNNHRNPWFRDFWEQKFQCSLQNKRNHRRVCEKHLAIDSSNYEQESKIMFVVNAVYAMAHALHKMQRTLCPNTTKLCDAMKILDGKKLYKDYLLKINFTAPFNPNKDADSIVKFDTFGDGMGRYNVFNFQNVGGKYSYLKVGHWAETLSLDVNSIHWSRNSVPTSQCSDPCAPNEMKNMQPGDVCCWICIPCEPYEYLADEFTCMDCGSGQWPTADLTGCYDLPEDYIRWEDAWAIGPVTIACLGFMCTCMVVTVFIKHNNTPLVKASGRELCYILLFGVGLSYCMTFFFIAKPSPVICALRRLGLGSSFAICYSALLTKTNCIARIFDGVKNGAQRPKFISPSSQVFICLGLISGQIVMVSVWLILEAPGTRRYTLAEKRETVILKCNVKDSSMLISLTYDVILVILCTVYAFKTRKCPENFNEAKFIGFTMYTTCIIWLAFLPIFYVTSSDYRVQTTTMCISVSLSGFVVLGCLFAPKVHIILFQPQKNVVTHRLHLNRFSVSGTGTTYSQSSASTYVPTVCNGREVLDSTTSSLT

SEQ ID NO: 38 - protein, 880 aa, Human
MKMLTRLQVLTLALFSKGFLLSLGDHNFLRREIKIEGDLVLGGLFPINEKGTGTEECGRINEDRGIQRLEAMLFAIDEINKDDYLLPGVKLGVHILDTCSRDTYALEQSLEFVRASLTKVDEAEYMCPDGSYAIQENIPLLIAGVIGGSYSSVSIQVANLLRLFQIPQISYASTSAKLSDKSRYDYFARTVPPDFYQAKAMAEILRFFNWTYVSTVASEGDYGETGIEAFEQEARLRNICIATAEKVGRSNIRKSYDSVIRELLQKPNARVVVLFMRSDDSRELIAAASRANASFTWVASDGWGAQESIIKGSEHVAYGDITLELASQPVRQFGRYFQSLNPYNNHRNPWFRDFWEQKFQCSLQNKRNHRRVCEKHLAIDSSNYEQESKIMFVVNAVYAMAHALHKMQRTLCPNTTKLCDAMKILDGKKLYKDYLLKINFTAPFNPNKDADSIVKFDTFGDGMGRYNVFNFQNVGGKYSYLKVGHWAETLSLDVNSIHWSRNSVPTSQCSDPCAPNEMKNMQPGDVCCWICIPCEPYEYLADEFTCMDCGSGQWPTADLTGCYDLPEDYIRWEDAWAIGPVTIACLGFMCTCMVVTVFIKHNNTPLVKASGRELCYILLFGVGLSYCMTFFFIAKPSPVICALRRLGLGSSFAICYSALLTKTNCIARIFDGVKNGAQRPKFISPSSQVFICLGLISVQIVMVSVWLILEAPGTRRYTLAEKRETVILKCNVKDSSMLISLTYNVILVILCTVYAFKTRKCPENFNEAKFIGFTMYTTCIIWLAFLPIFYVTSSDYRVQTTTMCISVSLSGFVVLGCLFAPKVHIILFQPQKNVVTHRLHLNRFSVSGTGTTYSQSSASTYVPTVCNGREVLDSTTSSLT

SEQ ID NO: 39 - protein, 880 aa, Human
MKMLTRLQVLTLALFSKGFLLSLGDHNFLRREIKIEGDLVLGGLFPINEKGTGTEECGRINEDRGIQRLEAMLFAIDEINKDDYLLPGVKLGVHILDTCSRDTYALEQSLEFVRASLTKVDEAEYMCPDGSYAIQENIPLLIAGVIGGSYSSVSIQVANLLRLFQIPQISYASTSAKLSDKSRYDYFARTVPPDFYQAKAMAEILRFFNWTYVSTVASEGDYGETGIEAFEQEARLRNICIATAEKVGRSNIRKSYDSVIRELLQKPNARVVVLFMRSDDSRELIAAASRANASFTWVASDGWGAQESIIKGSEHVAYGDITLELASQPVRQFGRYFQSLNPYNNHRNPWFRDFWEQKFQCSLQNKRNHRRVCEKHLAIDSSNYEQESKIMFVVNAVYAMAHALHKMQRTLCPNTTKLCDAMKILDGKKLYKDYLLKINFTAPFNPNKDADSIVKFDTFGDGMGRYNVFNFQNVGGKYSYLKVGHWAETLSLDVNSIHWSRNSVPTSQCSDPCAPNEMKNMQPGDVCCWICIPCEPYEYLADEFTCMDCGSGQWPTADLTGCYDLPEDYIRWEDAWAIGPVTIACLGFMCTCMVVTVFIKHNNTPLVKASGRELCYILLFGVGLSYCMTFFFIAKPSPVICALRRLGLGSSFAICYSALLTKTNCIARIFDGVKNGAQRPKFISPSSQVFICLGLILGQIVMVSVWLILEAPGTRRYTLAEKRETVILKCNVKDSSMLISLTYDVILVILCTVYAFKTRKCPENFNEAKFIGFTMYTTCIIWLAFLPIFYVTSSDYRVQTTTMCISVSLSGFVVLGCLFAPKVHIILFQPQKNVVTHRLHLNRFSVSGTGTTYSQSSASTYVPTVCNGREVLDSTTSSLT

SEQ ID NO: 40 - protein, 880 aa, Human
MKMLTRLQVLTLALFSKGFLLSLGDHNFLRREIKIEGDLVLGGLFPINEKGTGTEECGRINEDRGIQRLEAMLFAIDEINKDDYLLPGVKLGVHILDTCSRDTYALEQSLEFVRASLTKVDEAEYMCPDGSYAIQENIPLLIAGVIGGSYSSVSIQVANLLRLFQIPQISYASTSAKLSDKSRYDYFARTVPPDFYQAKAMAEILRFFNWTYVSTVASEGDYGETGIEAFEQEARLRNICIATAEKVGRSNIRKSYDSVIRELLQKPNARVVVLFMRSDDSRELIAAASRANASFTWVASDGWGAQESIIKGSEHVAYGDITLELASQPVRQFGRYFQSLNPYNNHRNPWFRDFWEQKFQCSLQNKRNHRRVCEKHLAIDSSNYEQESKIMFVVNAVYAMAHALHKMQRTLCPNTTKLCDAMKILDGKKLYKDYLLKINFTAPFNPNKDADSIVKFDTFGDGMGRYNVFNFQNVGGKYSYLKVGHWAETLSLDVNSIHWSRNSVPTSQCSDPCAPNEMKNMQPGDVCCWICIPCEPYEYLADEFTCMDCGSGQWPTADLTGCYDLPEDYIRWEDAWAIGPVTIACLGFMCTCMVVTVFIKHNNTPLVKASGRELCYILLFGVGLSYCMTFFFIAKPSPVICALRRLGLGSSFAICYSALLTKTNCIARIFDGVKNGAQRPKFISPSSQVFICLGLISGQIVMVSVWLILEAPGTRRYTLAEKRETVILKCNVKDSSMLISLAYNVILVILCTVYAFKTRKCPENFNEAKFIGFTMYTTCIIWLAFLPIFYVTSSDYRVQTTTMCISVSLSGFVVLGCLFAPKVHIILFQPQKNVVTHRLHLNRFSVSGTGTTYSQSSASTYVPTVCNGREVLDSTTSSLT

SEQ ID NO: 41 - DNA, 2640 bp, Human
atgaagatgttgacaagactgcaagttcttaccttagctttgttttcaaagggatttttactctctttaggggaccataactttctaaggagagagattaaaatagaaggtgaccttgttttagggggcctgtttcctattaacgaaaaaggcactggaactgaagaatgtgggcgaatcaatgaagaccgagggattcaacgcctggaagccatgttgtttgctattgatgaaatcaacaaagatgattacttgctaccaggagtgaagttgggtgttcacattttggatacatgttcaagggatacctatgcattggagcaatcactggagtttgtcagggcatctttgacaaaagtggatgaagctgagtatatgtgtcctgatggatcctatgccattcaagaaaacatcccacttctcattgcaggggtcattggtggctcttatagcagtgtttccatacaggtggcaaacctgctgcggctcttccagatccctcagatcagctacgcatccaccagcgccaaactcagtgataagtcgcgctatgattactttgccaggaccgtgccccccgacttctaccaggccaaagccatggctgagatcttgcgcttcttcaactggacctacgtgtccacagtagcctccgagggtgattacggggagacagggatcgaggccttcgagcaggaagcccgcctgcgcaacatctgcatcgctacggcggagaaggtgggccgctccaacatccgcaagtcctacgacagcgtgatccgagaactgttgcagaagcccaacgcgcgcgtcgtggtcctcttcatgcgcagcgacgactcgcgggagctcattgcagccgccagccgcgccaatgcctccttcacctgggtggccagcgacggttggggcgcgcaggagagcatcatcaagggcagcgagcatgtggcctacggcgacatcaccctggagctggcctcccagcctgtccgccagttcggccgctacttccagagcctcaacccctacaacaaccaccgcaacccctggttccgggacttctgggagcaaaagtttcagtgcagcctccagaacaaacgcaaccacaggcgcgtctgcgaaaagcacctggccatcgacagcagcaactacgagcaagagtccaagatcatgtttgtggtgaacgcggtgtatgccatggcccacgctttgcacaaaatgcagcgcaccctctgtcccaacactaccaagctttgtgatgctatgaagatcctggatgggaagaagttgtacaaggattacttgctgaaaatcaacttcacggctccattcaacccaaataaagatgcagatagcatagtcaagtttgacacttttggagatggaatggggcgatacaacgtgttcaatttccaaaatgtaggtgggaagtattcctacttgaaagttggtcactgggcagaaaccttatcgctagatgtcaactctatccactggtcccggaactcagtccccacttcccagtgcagcgacccctgtgcccccaatgaaatgaagaatatgcaaccaggggatgtctgctgctggatttgcatcccctgtgaaccctacgaatacctggctgatgagtttacctgtatggattgtgggtctggacagtggcccactgcagacctaactggatgctatgaccttcctgaggactacatcaggtgggaagacgcctgggccattggcccagtcaccattgcctgtctgggttttatgtgtacatgcatggttgtaactgtttttatcaagcacaacaacacacccttggtcaaagcatcgggccgagaactctgctacatcttattgtttggggttggcctgtcatactgcatgacattcttcttcattgccaagccatcaccagtcatctgtgcattgcgccgactcgggctggggagttccttcgctatctgttactcagccctgctgaccaagacaaactgcattgcccgcatcttcgatggggtcaagaatggcgctcagaggccaaaattcatcagccccagttctcaggttttcatctgcctgggtctgatcctggtgcaaattgtgatggtgtctgtgtggctcatcctggaggccccaggcaccaggaggtatacccttgcagagaagcgggaaacagtcatcctaaaatgcaatgtcaaagattccagcatgttgatctctcttacctacaatgtgatcctggtgatcttatgcactgtgtacgccttcaaaacgcggaagtgcccagaaaatttcaacgaagctaagttcataggttttaccatgtacaccacgtgcatcatctggttggccttcctccctatattttatgtgacatcaagtgactacagagtgcagacgacaaccatgtgcatctctgtcagcctgagtggctttgtggtcttgggctgtttgtttgcacccaaggttcacatcatcctgtttcaaccccagaagaatgttgtcacacacagactgcacctcaacaggttcagtgtcagtggaactgggaccacatactctcagtcctctgcaagcacgtatgtgccaacggtgtgcaatgggcgggaagtcctcgactccaccacctcatctctgtga

SEQ ID NO: 42 - DNA, 2640 bp, Human
atgaagatgttgacaagactgcaagttcttaccttagctttgttttcaaagggatttttactctctttaggggaccataactttctaaggagagagattaaaatagaaggtgaccttgttttagggggcctgtttcctattaacgaaaaaggcactggaactgaagaatgtgggcgaatcaatgaagaccgagggattcaacgcctggaagccatgttgtttgctattgatgaaatcaacaaagatgattacttgctaccaggagtgaagttgggtgttcacattttggatacatgttcaagggatacctatgcattggagcaatcactggagtttgtcagggcatctttgacaaaagtggatgaagctgagtatatgtgtcctgatggatcctatgccattcaagaaaacatcccacttctcattgcaggggtcattggtggctcttatagcagtgtttccatacaggtggcaaacctgctgcggctcttccagatccctcagatcagctacgcatccaccagcgccaaactcagtgataagtcgcgctatgattactttgccaggaccgtgccccccgacttctaccaggccaaagccatggctgagatcttgcgcttcttcaactggacctacgtgtccacagtagcctccgagggtgattacggggagacagggatcgaggccttcgagcaggaagcccgcctgcgcaacatctgcatcgctacggcggagaaggtgggccgctccaacatccgcaagtcctacgacagcgtgatccgagaactgttgcagaagcccaacgcgcgcgtcgtggtcctcttcatgcgcagcgacgactcgcgggagctcattgcagccgccagccgcgccaatgcctccttcacctgggtggccagcgacggttggggcgcgcaggagagcatcatcaagggcagcgagcatgtggcctacggcgacatcaccctggagctggcctcccagcctgtccgccagttcggccgctacttccagagcctcaacccctacaacaaccaccgcaacccctggttccgggacttctgggagcaaaagtttcagtgcagcctccagaacaaacgcaaccacaggcgcgtctgcgaaaagcacctggccatcgacagcagcaactacgagcaagagtccaagatcatgtttgtggtgaacgcggtgtatgccatggcccacgctttgcacaaaatgcagcgcaccctctgtcccaacactaccaagctttgtgatgctatgaagatcctggatgggaagaagttgtacaaggattacttgctgaaaatcaacttcacggctccattcaacccaaataaagatgcagatagcatagtcaagtttgacacttttggagatggaatggggcgatacaacgtgttcaatttccaaaatgtaggtgggaagtattcctacttgaaagttggtcactgggcagaaaccttatcgctagatgtcaactctatccactggtcccggaactcagtccccacttcccagtgcagcgacccctgtgcccccaatgaaatgaagaatatgcaaccaggggatgtctgctgctggatttgcatcccctgtgaaccctacgaatacctggctgatgagtttacctgtatggattgtgggtctggacagtggcccactgcagacctaactggatgctatgaccttcctgaggactacatcaggtgggaagacgcctgggccattggcccagtcaccattgcctgtctgggttttatgtgtacatgcatggttgtaactgtttttatcaagcacaacaacacacccttggtcaaagcatcgggccgagaactctgctacatcttattgtttggggttggcctgtcatactgcatgacattcttcttcattgccaagccatcaccagtcatctgtgcattgcgccgactcgggctggggagttccttcgctatctgttactcagccctgctgaccaagacaaactgcattgcccgcatcttcgatggggtcaagaatggcgctcagaggccaaaattcatcagccccagttctcaggttttcatctgcctgggtctgatcctggggcaaattgtgatggtgtctgtgtggctcatcctggaggccccaggcaccaggaggtatacccttgcagagaagcgggaaacagtcatcctaaaatgcaatgtcaaagattccagcatgttgatctctcttacctacaatgtgatcctggtgatcttatgcactgtgtacgccttcaaaacgcggaagtgcccagaaaatttcaacgaagctaagttcataggttttaccatgtacaccacgtgcatcatctggttggccttcctccctatattttatgtgacatcaagtgactacagagtgcagacgacaaccatgtgcatctctgtcagcctgagtggctttgtggtcttgggctgtttgtttgcacccaaggttcacatcatcctgtttcaaccccagaagaatgttgtcacacacagactgcacctcaacaggttcagtgtcagtggaactgggaccacatactctcagtcctctgcaagcacgtatgtgccaacggtgtgcaatgggcgggaagtcctcgactccaccacctcatctctgtga

SEQ ID NO: 43 - DNA, 2640 bp, Human
atgaagatgttgacaagactgcaagttcttaccttagctttgttttcaaagggatttttactctctttaggggaccataactttctaaggagagagattaaaatagaaggtgaccttgttttagggggcctgtttcctattaacgaaaaaggcactggaactgaagaatgtgggcgaatcaatgaagaccgagggattcaacgcctggaagccatgttgtttgctattgatgaaatcaacaaagatgattacttgctaccaggagtgaagttgggtgttcacattttggatacatgttcaagggatacctatgcattggagcaatcactggagtttgtcagggcatctttgacaaaagtggatgaagctgagtatatgtgtcctgatggatcctatgccattcaagaaaacatcccacttctcattgcaggggtcattggtggctcttatagcagtgtttccatacaggtggcaaacctgctgcggctcttccagatccctcagatcagctacgcatccaccagcgccaaactcagtgataagtcgcgctatgattactttgccaggaccgtgccccccgacttctaccaggccaaagccatggctgagatcttgcgcttcttcaactggacctacgtgtccacagtagcctccgagggtgattacggggagacagggatcgaggccttcgagcaggaagcccgcctgcgcaacatctgcatcgctacggcggagaaggtgggccgctccaacatccgcaagtcctacgacagcgtgatccgagaactgttgcagaagcccaacgcgcgcgtcgtggtcctcttcatgcgcagcgacgactcgcgggagctcattgcagccgccagccgcgccaatgcctccttcacctgggtggccagcgacggttggggcgcgcaggagagcatcatcaagggcagcgagcatgtggcctacggcgacatcaccctggagctggcctcccagcctgtccgccagttcggccgctacttccagagcctcaacccctacaacaaccaccgcaacccctggttccgggacttctgggagcaaaagtttcagtgcagcctccagaacaaacgcaaccacaggcgcgtctgcgaaaagcacctggccatcgacagcagcaactacgagcaagagtccaagatcatgtttgtggtgaacgcggtgtatgccatggcccacgctttgcacaaaatgcagcgcaccctctgtcccaacactaccaagctttgtgatgctatgaagatcctggatgggaagaagttgtacaaggattacttgctgaaaatcaacttcacggctccattcaacccaaataaagatgcagatagcatagtcaagtttgacacttttggagatggaatggggcgatacaacgtgttcaatttccaaaatgtaggtgggaagtattcctacttgaaagttggtcactgggcagaaaccttatcgctagatgtcaactctatccactggtcccggaactcagtccccacttcccagtgcagcgacccctgtgcccccaatgaaatgaagaatatgcaaccaggggatgtctgctgctggatttgcatcccctgtgaaccctacgaatacctggctgatgagtttacctgtatggattgtgggtctggacagtggcccactgcagacctaactggatgctatgaccttcctgaggactacatcaggtgggaagacgcctgggccattggcccagtcaccattgcctgtctgggttttatgtgtacatgcatggttgtaactgtttttatcaagcacaacaacacacccttggtcaaagcatcgggccgagaactctgctacatcttattgtttggggttggcctgtcatactgcatgacattcttcttcattgccaagccatcaccagtcatctgtgcattgcgccgactcgggctggggagttccttcgctatctgttactcagccctgctgaccaagacaaactgcattgcccgcatcttcgatggggtcaagaatggcgctcagaggccaaaattcatcagccccagttctcaggttttcatctgcctgggtctgatctcggggcaaattgtgatggtgtctgtgtggctcatcctggaggccccaggcaccaggaggtatacccttgcagagaagcgggaaacagtcatcctaaaatgcaatgtcaaagattccagcatgttgatctctcttacctacaatgtgatcctggtgatcttatgcactgtgtacgccttcaaaacgcggaagtgcccagaaaatttcaacgaagctaagttcataggttttaccatgtacaccacgtgcatcatctggttggccttcctccctatattttatgtgacatcaagtgactacagagtgcagacgacaaccatgtgcatctctgtcagcctgagtggctttgtggtcttgggctgtttgtttgcacccaaggttcacatcatcctgtttcaaccccagaagaatgttgtcacacacagactgcacctcaacaggttcagtgtcagtggaactgggaccacatactctcagtcctctgcaagcacgtatgtgccaacggtgtgcaatgggcgggaagtcctcgactccaccacctcatctctgtga

SEQ ID NO: 44 - DNA, 2640 bp, Human
atgaagatgttgacaagactgcaagttcttaccttagctttgttttcaaagggatttttactctctttaggggaccataactttctaaggagagagattaaaatagaaggtgaccttgttttagggggcctgtttcctattaacgaaaaaggcactggaactgaagaatgtgggcgaatcaatgaagaccgagggattcaacgcctggaagccatgttgtttgctattgatgaaatcaacaaagatgattacttgctaccaggagtgaagttgggtgttcacattttggatacatgttcaagggatacctatgcattggagcaatcactggagtttgtcagggcatctttgacaaaagtggatgaagctgagtatatgtgtcctgatggatcctatgccattcaagaaaacatcccacttctcattgcaggggtcattggtggctcttatagcagtgtttccatacaggtggcaaacctgctgcggctcttccagatccctcagatcagctacgcatccaccagcgccaaactcagtgataagtcgcgctatgattactttgccaggaccgtgccccccgacttctaccaggccaaagccatggctgagatcttgcgcttcttcaactggacctacgtgtccacagtagcctccgagggtgattacggggagacagggatcgaggccttcgagcaggaagcccgcctgcgcaacatctgcatcgctacggcggagaaggtgggccgctccaacatccgcaagtcctacgacagcgtgatccgagaactgttgcagaagcccaacgcgcgcgtcgtggtcctcttcatgcgcagcgacgactcgcgggagctcattgcagccgccagccgcgccaatgcctccttcacctgggtggccagcgacggttggggcgcgcaggagagcatcatcaagggcagcgagcatgtggcctacggcgacatcaccctggagctggcctcccagcctgtccgccagttcggccgctacttccagagcctcaacccctacaacaaccaccgcaacccctggttccgggacttctgggagcaaaagtttcagtgcagcctccagaacaaacgcaaccacaggcgcgtctgcgaaaagcacctggccatcgacagcagcaactacgagcaagagtccaagatcatgtttgtggtgaacgcggtgtatgccatggcccacgctttgcacaaaatgcagcgcaccctctgtcccaacactaccaagctttgtgatgctatgaagatcctggatgggaagaagttgtacaaggattacttgctgaaaatcaacttcacggctccattcaacccaaataaagatgcagatagcatagtcaagtttgacacttttggagatggaatggggcgatacaacgtgttcaatttccaaaatgtaggtgggaagtattcctacttgaaagttggtcactgggcagaaaccttatcgctagatgtcaactctatccactggtcccggaactcagtccccacttcccagtgcagcgacccctgtgcccccaatgaaatgaagaatatgcaaccaggggatgtctgctgctggatttgcatcccctgtgaaccctacgaatacctggctgatgagtttacctgtatggattgtgggtctggacagtggcccactgcagacctaactggatgctatgaccttcctgaggactacatcaggtgggaagacgcctgggccattggcccagtcaccattgcctgtctgggttttatgtgtacatgcatggttgtaactgtttttatcaagcacaacaacacacccttggtcaaagcatcgggccgagaactctgctacatcttattgtttggggttggcctgtcatactgcatgacattcttcttcattgccaagccatcaccagtcatctgtgcattgcgccgactcgggctggggagttccttcgctatctgttactcagccctgctgaccaagacaaactgcattgcccgcatcttcgatggggtcaagaatggcgctcagaggccaaaattcatcagccccagttctcaggttttcatctgcctgggtctgatctcggtgcaaattgtgatggtgtctgtgtggctcatcctggaggccccaggcaccaggaggtatacccttgcagagaagcgggaaacagtcatcctaaaatgcaatgtcaaagattccagcatgttgatctctcttacctacgatgtgatcctggtgatcttatgcactgtgtacgccttcaaaacgcggaagtgcccagaaaatttcaacgaagctaagttcataggttttaccatgtacaccacgtgcatcatctggttggccttcctccctatattttatgtgacatcaagtgactacagagtgcagacgacaaccatgtgcatctctgtcagcctgagtggctttgtggtcttgggctgtttgtttgcacccaaggttcacatcatcctgtttcaaccccagaagaatgttgtcacacacagactgcacctcaacaggttcagtgtcagtggaactgggaccacatactctcagtcctctgcaagcacgtatgtgccaacggtgtgcaatgggcgggaagtcctcgactccaccacctcatctctgtga

SEQ ID NO: 45 - DNA, 2640 bp, Human
atgaagatgttgacaagactgcaagttcttaccttagctttgttttcaaagggatttttactctctttaggggaccataactttctaaggagagagattaaaatagaaggtgaccttgttttagggggcctgtttcctattaacgaaaaaggcactggaactgaagaatgtgggcgaatcaatgaagaccgagggattcaacgcctggaagccatgttgtttgctattgatgaaatcaacaaagatgattacttgctaccaggagtgaagttgggtgttcacattttggatacatgttcaagggatacctatgcattggagcaatcactggagtttgtcagggcatctttgacaaaagtggatgaagctgagtatatgtgtcctgatggatcctatgccattcaagaaaacatcccacttctcattgcaggggtcattggtggctcttatagcagtgtttccatacaggtggcaaacctgctgcggctcttccagatccctcagatcagctacgcatccaccagcgccaaactcagtgataagtcgcgctatgattactttgccaggaccgtgccccccgacttctaccaggccaaagccatggctgagatcttgcgcttcttcaactggacctacgtgtccacagtagcctccgagggtgattacggggagacagggatcgaggccttcgagcaggaagcccgcctgcgcaacatctgcatcgctacggcggagaaggtgggccgctccaacatccgcaagtcctacgacagcgtgatccgagaactgttgcagaagcccaacgcgcgcgtcgtggtcctcttcatgcgcagcgacgactcgcgggagctcattgcagccgccagccgcgccaatgcctccttcacctgggtggccagcgacggttggggcgcgcaggagagcatcatcaagggcagcgagcatgtggcctacggcgacatcaccctggagctggcctcccagcctgtccgccagttcggccgctacttccagagcctcaacccctacaacaaccaccgcaacccctggttccgggacttctgggagcaaaagtttcagtgcagcctccagaacaaacgcaaccacaggcgcgtctgcgaaaagcacctggccatcgacagcagcaactacgagcaagagtccaagatcatgtttgtggtgaacgcggtgtatgccatggcccacgctttgcacaaaatgcagcgcaccctctgtcccaacactaccaagctttgtgatgctatgaagatcctggatgggaagaagttgtacaaggattacttgctgaaaatcaacttcacggctccattcaacccaaataaagatgcagatagcatagtcaagtttgacacttttggagatggaatggggcgatacaacgtgttcaatttccaaaatgtaggtgggaagtattcctacttgaaagttggtcactgggcagaaaccttatcgctagatgtcaactctatccactggtcccggaactcagtccccacttcccagtgcagcgacccctgtgcccccaatgaaatgaagaatatgcaaccaggggatgtctgctgctggatttgcatcccctgtgaaccctacgaatacctggctgatgagtttacctgtatggattgtgggtctggacagtggcccactgcagacctaactggatgctatgaccttcctgaggactacatcaggtgggaagacgcctgggccattggcccagtcaccattgcctgtctgggttttatgtgtacatgcatggttgtaactgtttttatcaagcacaacaacacacccttggtcaaagcatcgggccgagaactctgctacatcttattgtttggggttggcctgtcatactgcatgacattcttcttcattgccaagccatcaccagtcatctgtgcattgcgccgactcgggctggggagttccttcgctatctgttactcagccctgctgaccaagacaaactgcattgcccgcatcttcgatggggtcaagaatggcgctcagaggccaaaattcatcagccccagttctcaggttttcatctgcctgggtctgatctcggggcaaattgtgatggtgtctgtgtggctcatcctggaggccccaggcaccaggaggtatacccttgcagagaagcgggaaacagtcatcctaaaatgcaatgtcaaagattccagcatgttgatctctcttacctacgatgtgatcctggtgatcttatgcactgtgtacgccttcaaaacgcggaagtgcccagaaaatttcaacgaagctaagttcataggttttaccatgtacaccacgtgcatcatctggttggccttcctccctatattttatgtgacatcaagtgactacagagtgcagacgacaaccatgtgcatctctgtcagcctgagtggctttgtggtcttgggctgtttgtttgcacccaaggttcacatcatcctgtttcaaccccagaagaatgttgtcacacacagactgcacctcaacaggttcagtgtcagtggaactgggaccacatactctcagtcctctgcaagcacgtatgtgccaacggtgtgcaatgggcgggaagtcctcgactccaccacctcatctctgtga

SEQ ID NO: 46 - DNA, 2640 bp, Human
atgaagatgttgacaagactgcaagttcttaccttagctttgttttcaaagggatttttactctctttaggggaccataactttctaaggagagagattaaaatagaaggtgaccttgttttagggggcctgtttcctattaacgaaaaaggcactggaactgaagaatgtgggcgaatcaatgaagaccgagggattcaacgcctggaagccatgttgtttgctattgatgaaatcaacaaagatgattacttgctaccaggagtgaagttgggtgttcacattttggatacatgttcaagggatacctatgcattggagcaatcactggagtttgtcagggcatctttgacaaaagtggatgaagctgagtatatgtgtcctgatggatcctatgccattcaagaaaacatcccacttctcattgcaggggtcattggtggctcttatagcagtgtttccatacaggtggcaaacctgctgcggctcttccagatccctcagatcagctacgcatccaccagcgccaaactcagtgataagtcgcgctatgattactttgccaggaccgtgccccccgacttctaccaggccaaagccatggctgagatcttgcgcttcttcaactggacctacgtgtccacagtagcctccgagggtgattacggggagacagggatcgaggccttcgagcaggaagcccgcctgcgcaacatctgcatcgctacggcggagaaggtgggccgctccaacatccgcaagtcctacgacagcgtgatccgagaactgttgcagaagcccaacgcgcgcgtcgtggtcctcttcatgcgcagcgacgactcgcgggagctcattgcagccgccagccgcgccaatgcctccttcacctgggtggccagcgacggttggggcgcgcaggagagcatcatcaagggcagcgagcatgtggcctacggcgacatcaccctggagctggcctcccagcctgtccgccagttcggccgctacttccagagcctcaacccctacaacaaccaccgcaacccctggttccgggacttctgggagcaaaagtttcagtgcagcctccagaacaaacgcaaccacaggcgcgtctgcgaaaagcacctggccatcgacagcagcaactacgagcaagagtccaagatcatgtttgtggtgaacgcggtgtatgccatggcccacgctttgcacaaaatgcagcgcaccctctgtcccaacactaccaagctttgtgatgctatgaagatcctggatgggaagaagttgtacaaggattacttgctgaaaatcaacttcacggctccattcaacccaaataaagatgcagatagcatagtcaagtttgacacttttggagatggaatggggcgatacaacgtgttcaatttccaaaatgtaggtgggaagtattcctacttgaaagttggtcactgggcagaaaccttatcgctagatgtcaactctatccactggtcccggaactcagtccccacttcccagtgcagcgacccctgtgcccccaatgaaatgaagaatatgcaaccaggggatgtctgctgctggatttgcatcccctgtgaaccctacgaatacctggctgatgagtttacctgtatggattgtgggtctggacagtggcccactgcagacctaactggatgctatgaccttcctgaggactacatcaggtgggaagacgcctgggccattggcccagtcaccattgcctgtctgggttttatgtgtacatgcatggttgtaactgtttttatcaagcacaacaacacacccttggtcaaagcatcgggccgagaactctgctacatcttattgtttggggttggcctgtcatactgcatgacattcttcttcattgccaagccatcaccagtcatctgtgcattgcgccgactcgggctggggagttccttcgctatctgttactcagccctgctgaccaagacaaactgcattgcccgcatcttcgatggggtcaagaatggcgctcagaggccaaaattcatcagccccagttctcaggttttcatctgcctgggtctgatctcggtgcaaattgtgatggtgtctgtgtggctcatcctggaggccccaggcaccaggaggtatacccttgcagagaagcgggaaacagtcatcctaaaatgcaatgtcaaagattccagcatgttgatctctcttacctacaatgtgatcctggtgatcttatgcactgtgtacgccttcaaaacgcggaagtgcccagaaaatttcaacgaagctaagttcataggttttaccatgtacaccacgtgcatcatctggttggccttcctccctatattttatgtgacatcaagtgactacagagtgcagacgacaaccatgtgcatctctgtcagcctgagtggctttgtggtcttgggctgtttgtttgcacccaaggttcacatcatcctgtttcaaccccagaagaatgttgtcacacacagactgcacctcaacaggttcagtgtcagtggaactgggaccacatactctcagtcctctgcaagcacgtatgtgccaacggtgtgcaatgggcgggaagtcctcgactccaccacctcatctctgtga

SEQ ID NO: 47 - DNA, 2640 bp, Human
atgaagatgttgacaagactgcaagttcttaccttagctttgttttcaaagggatttttactctctttaggggaccataactttctaaggagagagattaaaatagaaggtgaccttgttttagggggcctgtttcctattaacgaaaaaggcactggaactgaagaatgtgggcgaatcaatgaagaccgagggattcaacgcctggaagccatgttgtttgctattgatgaaatcaacaaagatgattacttgctaccaggagtgaagttgggtgttcacattttggatacatgttcaagggatacctatgcattggagcaatcactggagtttgtcagggcatctttgacaaaagtggatgaagctgagtatatgtgtcctgatggatcctatgccattcaagaaaacatcccacttctcattgcaggggtcattggtggctcttatagcagtgtttccatacaggtggcaaacctgctgcggctcttccagatccctcagatcagctacgcatccaccagcgccaaactcagtgataagtcgcgctatgattactttgccaggaccgtgccccccgacttctaccaggccaaagccatggctgagatcttgcgcttcttcaactggacctacgtgtccacagtagcctccgagggtgattacggggagacagggatcgaggccttcgagcaggaagcccgcctgcgcaacatctgcatcgctacggcggagaaggtgggccgctccaacatccgcaagtcctacgacagcgtgatccgagaactgttgcagaagcccaacgcgcgcgtcgtggtcctcttcatgcgcagcgacgactcgcgggagctcattgcagccgccagccgcgccaatgcctccttcacctgggtggccagcgacggttggggcgcgcaggagagcatcatcaagggcagcgagcatgtggcctacggcgacatcaccctggagctggcctcccagcctgtccgccagttcggccgctacttccagagcctcaacccctacaacaaccaccgcaacccctggttccgggacttctgggagcaaaagtttcagtgcagcctccagaacaaacgcaaccacaggcgcgtctgcgaaaagcacctggccatcgacagcagcaactacgagcaagagtccaagatcatgtttgtggtgaacgcggtgtatgccatggcccacgctttgcacaaaatgcagcgcaccctctgtcccaacactaccaagctttgtgatgctatgaagatcctggatgggaagaagttgtacaaggattacttgctgaaaatcaacttcacggctccattcaacccaaataaagatgcagatagcatagtcaagtttgacacttttggagatggaatggggcgatacaacgtgttcaatttccaaaatgtaggtgggaagtattcctacttgaaagttggtcactgggcagaaaccttatcgctagatgtcaactctatccactggtcccggaactcagtccccacttcccagtgcagcgacccctgtgcccccaatgaaatgaagaatatgcaaccaggggatgtctgctgctggatttgcatcccctgtgaaccctacgaatacctggctgatgagtttacctgtatggattgtgggtctggacagtggcccactgcagacctaactggatgctatgaccttcctgaggactacatcaggtgggaagacgcctgggccattggcccagtcaccattgcctgtctgggttttatgtgtacatgcatggttgtaactgtttttatcaagcacaacaacacacccttggtcaaagcatcgggccgagaactctgctacatcttattgtttggggttggcctgtcatactgcatgacattcttcttcattgccaagccatcaccagtcatctgtgcattgcgccgactcgggctggggagttccttcgctatctgttactcagccctgctgaccaagacaaactgcattgcccgcatcttcgatggggtcaagaatggcgctcagaggccaaaattcatcagccccagttctcaggttttcatctgcctgggtctgatcctggggcaaattgtgatggtgtctgtgtggctcatcctggaggccccaggcaccaggaggtatacccttgcagagaagcgggaaacagtcatcctaaaatgcaatgtcaaagattccagcatgttgatctctcttacctacgatgtgatcctggtgatcttatgcactgtgtacgccttcaaaacgcggaagtgcccagaaaatttcaacgaagctaagttcataggttttaccatgtacaccacgtgcatcatctggttggccttcctccctatattttatgtgacatcaagtgactacagagtgcagacgacaaccatgtgcatctctgtcagcctgagtggctttgtggtcttgggctgtttgtttgcacccaaggttcacatcatcctgtttcaaccccagaagaatgttgtcacacacagactgcacctcaacaggttcagtgtcagtggaactgggaccacatactctcagtcctctgcaagcacgtatgtgccaacggtgtgcaatgggcgggaagtcctcgactccaccacctcatctctgtga

SEQ ID NO: 48 - DNA, 2640 bp, Human
atgaagatgttgacaagactgcaagttcttaccttagctttgttttcaaagggatttttactctctttaggggaccataactttctaaggagagagattaaaatagaaggtgaccttgttttagggggcctgtttcctattaacgaaaaaggcactggaactgaagaatgtgggcgaatcaatgaagaccgagggattcaacgcctggaagccatgttgtttgctattgatgaaatcaacaaagatgattacttgctaccaggagtgaagttgggtgttcacattttggatacatgttcaagggatacctatgcattggagcaatcactggagtttgtcagggcatctttgacaaaagtggatgaagctgagtatatgtgtcctgatggatcctatgccattcaagaaaacatcccacttctcattgcaggggtcattggtggctcttatagcagtgtttccatacaggtggcaaacctgctgcggctcttccagatccctcagatcagctacgcatccaccagcgccaaactcagtgataagtcgcgctatgattactttgccaggaccgtgccccccgacttctaccaggccaaagccatggctgagatcttgcgcttcttcaactggacctacgtgtccacagtagcctccgagggtgattacggggagacagggatcgaggccttcgagcaggaagcccgcctgcgcaacatctgcatcgctacggcggagaaggtgggccgctccaacatccgcaagtcctacgacagcgtgatccgagaactgttgcagaagcccaacgcgcgcgtcgtggtcctcttcatgcgcagcgacgactcgcgggagctcattgcagccgccagccgcgccaatgcctccttcacctgggtggccagcgacggttggggcgcgcaggagagcatcatcaagggcagcgagcatgtggcctacggcgacatcaccctggagctggcctcccagcctgtccgccagttcggccgctacttccagagcctcaacccctacaacaaccaccgcaacccctggttccgggacttctgggagcaaaagtttcagtgcagcctccagaacaaacgcaaccacaggcgcgtctgcgaaaagcacctggccatcgacagcagcaactacgagcaagagtccaagatcatgtttgtggtgaacgcggtgtatgccatggcccacgctttgcacaaaatgcagcgcaccctctgtcccaacactaccaagctttgtgatgctatgaagatcctggatgggaagaagttgtacaaggattacttgctgaaaatcaacttcacggctccattcaacccaaataaagatgcagatagcatagtcaagtttgacacttttggagatggaatggggcgatacaacgtgttcaatttccaaaatgtaggtgggaagtattcctacttgaaagttggtcactgggcagaaaccttatcgctagatgtcaactctatccactggtcccggaactcagtccccacttcccagtgcagcgacccctgtgcccccaatgaaatgaagaatatgcaaccaggggatgtctgctgctggatttgcatcccctgtgaaccctacgaatacctggctgatgagtttacctgtatggattgtgggtctggacagtggcccactgcagacctaactggatgctatgaccttcctgaggactacatcaggtgggaagacgcctgggccattggcccagtcaccattgcctgtctgggttttatgtgtacatgcatggttgtaactgtttttatcaagcacaacaacacacccttggtcaaagcatcgggccgagaactctgctacatcttattgtttggggttggcctgtcatactgcatgacattcttcttcattgccaagccatcaccagtcatctgtgcattgcgccgactcgggctggggagttccttcgctatctgttactcagccctgctgaccaagacaaactgcattgcccgcatcttcgatggggtcaagaatggcgctcagaggccaaaattcatcagccccagttctcaggttttcatctgcctgggtctgatctcggggcaaattgtgatggtgtctgtgtggctcatcctggaggccccaggcaccaggaggtatacccttgcagagaagcgggaaacagtcatcctaaaatgcaatgtcaaagattccagcatgttgatctctcttgcctacaatgtgatcctggtgatcttatgcactgtgtacgccttcaaaacgcggaagtgcccagaaaatttcaacgaagctaagttcataggttttaccatgtacaccacgtgcatcatctggttggccttcctccctatattttatgtgacatcaagtgactacagagtgcagacgacaaccatgtgcatctctgtcagcctgagtggctttgtggtcttgggctgtttgtttgcacccaaggttcacatcatcctgtttcaaccccagaagaatgttgtcacacacagactgcacctcaacaggttcagtgtcagtggaactgggaccacatactctcagtcctctgcaagcacgtatgtgccaacggtgtgcaatgggcgggaagtcctcgactccaccacctcatctctgtga

(19) United States
(12) Patent Application Publication
(10) Pub. No.: US 2006/0147923 A1
(43) Pub. Date: Jul. 6, 2006

(54) Nucleic acid sequences encoding novel point mutations on mglur2 and mglur3

(76) Inventors: Lorrie Patricia Daggett, San Diego, CA (US); Sylvia Morales, San Diego, CA (US); Herve Schaffhauser, San Diego, CA (US)

Correspondence Address: MERCK AND CO., INC, P O BOX 2000, RAHWAY, NJ, 07065-0907, US

(21) Appl. No.: 10/526,978

(22) PCT Filed: Sep. 11, 2003
(86) PCT No.: PCT/US03/28470

§ 371 Date: Mar. 7, 2005

Related U.S. Application Data
(60) Provisional application No. 60/409,910, filed on Sep. 11, 2002.

(51) Int. Cl. C12Q 1/68; C07H 21/04; C12P 21/06; C07K 14/705

(52) U.S. Cl. 435/6; 435/69.1; 435/320.1; 435/325; 530/350; 536/23.5

(57) ABSTRACT

The present invention is directed to mutant forms of mGluR2 and mGluR3 that are shown to affect the binding of modulators of said receptors. The invention discloses polypeptides comprising the mutations, and nucleic acid sequences that encodes said polypeptides, and methods of using said polypeptides and nucleic acids sequences to identify, predict and evaluate specific, selective modulators whose association to mGlu2 or mGlur3 is effected by said mutations.

### CROSS-REFERENCE TO RELATED APPLICATIONS

This application claims the benefit of U.S. Provisional Application No. 60/60/409,910, filed Sep. 9, 2002, the contents of which are incorporated herein by reference in their entirety.

### STATEMENT REGARDING FEDERALLY-SPONSORED R&D

Non applicable.

### REFERENCE TO MICROFICHE APPENDIX

Not applicable.

### BACKGROUND OF THE INVENTION

In the mammalian central nervous system (CNS), the transmission of nerve impulses is controlled by the interaction between a neurotransmitter, released by a sending neuron, and a surface receptor on a receiving neuron. This interaction causes excitation of the receiving neuron. L-Glutamate, which is the most abundant neurotransmitter in the CNS, mediates the major excitatory pathway in mammals, and is referred to as an excitatory amino acid (EAA). The receptors that respond to glutamate are called excitatory amino acid receptors (EAA receptors). See Watkins & Evans, Annual Reviews in Pharmacology and Toxicology, 21:165 (1981); Monaghan, Bridges, and Cotman, Annual Reviews in Pharmacology and Toxicology, 29:365 (1989); Watkins, Krogsgaard-Larsen, and Honore, Transactions in Pharmaceutical Science, 11:25 (1990). The excitatory amino acids are of great physiological importance, playing a role in a variety of physiological processes, such as long-term potentiation (learning and memory), the development of synaptic plasticity, motor control, respiration, cardiovascular regulation, and sensory perception.

Excitatory amino acid receptors are classified into two general families—ionotropic and metabotropic. Ionotropic receptors are directly coupled to the opening of cation channels in the cell membrane of the neurons. This type of receptor has been subdivided into at least four subtypes, which are defined by the depolarizing actions of the selective agonists γ-aminobutyric acid (GABA), N-methyl-D-aspartate (NMDA), α-amino-3-hydroxy-5-methylisoxazole-4-propionic acid (AMPA), and nicotinic acetylcholine (ACh). Kandel et al., Principles of Neural Science, 3d ed., Elsevier Science Press, 1991.

Metabotropic, the second general EAA receptor family, includes the G-protein (guanosine nucleotide-binding protein) or second messenger-linked “metabotropic” excitatory amino acid receptor (also known as a “G Protein-Coupled Receptor”, or a “GPCR”). This second type is coupled to multiple second messenger systems that lead to enhanced phosphoinositide hydrolysis, activation of phospholipase D, increases or decreases in cAMP formation, or changes in ion channel function. Schoepp and Conn, Trends in Pharmacological Science, 14:13 (1993). Both types of receptors appear not only to mediate normal synaptic transmission along excitatory pathways, but also participate in the modification of synaptic connections during development and throughout life. Schoepp, Bockaert, and Sladeczek, Trends in Pharmacological Science, 11:508 (1990); McDonald and Johnson, Brain Research Reviews, 15:41 (1990).

The excessive or inappropriate stimulation of excitatory amino acid receptors leads to neuronal cell damage or loss by way of a mechanism known as excitotoxicity. This process has been suggested to mediate neuronal degeneration in a variety of conditions. The medical consequences of such neuronal degeneration makes the abatement of these degenerative neurological processes an important therapeutic goal. In that one effect of a positive mGluR2 modulator (e.g., an mGluR2 potentiator) is to decrease activity at a synapse near mGluR2 receptors (by stimulating mGluR2 activity), the development of specific positive modulators for mGluR2 appears to be an appropriate approach to control neuronal pathways and, in doing so, reducing neural cell damage. A key to the specificity, and expected greater effectiveness, of such modulators, whether for mGluR2, mGluR3, or other GPCRS, is to be able to evaluate and identify modulators that, at a pharmacologically acceptable and effective dose, have a positive effect on one such GPCR, while not having the same positive effect on other GPCRs which have opposite or undesired effects on the cell.

In this regard, it is noted that the metabotropic glutamate receptors (“mGluRx,” where x is an integer) are a highly heterogeneous family of glutamate receptors. All are linked to multiple second-messenger pathways. Presently at least eight identified subtypes of such receptors that fall into three classes based on second-messenger association, sequence homology, and agonist selectivity. See BOND, A. et al., “Neuroprotective Effects of LY379268, a Selective mGlu2/3 Receptor Agonist: Investigations into Possible Mechanism of Action In Vivo”, JPET, 2000, 800-809: 294, USA. Also see “G Protein-Coupled Receptor Allosterism and Complexing” Pharmacological Reviews, 54:2, 323-374. These receptors function to modulate the presynaptic release of glutamate, and the postsynaptic sensitivity of the neuronal cell to glutamate excitation. It is generally recognized that agonists and antagonists of these receptors may be useful for the treatment of acute and chronic neurodegenerative conditions, and as antipsychotic, anticonvulsant, analgesic, anxiolytic, antidepressant, and anti-emetic agents. The present invention discloses at least one allosteric binding site which would be useful for the identification and development of mGluR2- and mGluR3-specific modulators that associate with such site.

In recent years, the role of allosteric modulators for the metabotropic glutamate receptors has been a subject of increasing interest and research. An advantage of developing a specific allosteric modulator for one type of metabotropic glutamate receptor is that the use of such modulator may provide a desirable pharmacological and behavioral result at lower doses, with less toxicity and side effects due to its specificity.

The present invention advances the art by identifying a site of allosteric modulation in mGluR2 and mGluR3, and by characterizing critical components of said site, through point mutations, where such mutations have a dramatic effect on potentiation of the glutamate cell receptor.

### SUMMARY OF THE INVENTION

The present invention provides novel forms of mGluR2 and mGluR3 comprising at least one allosteric binding site which has been modified by one, two, or three single amino acid point mutations. The loss of potentiation of receptors bearing such mutations in mGluR2 is clear and dramatic. The present invention provides nucleic acid sequences, expressible in isolated cells, useful for the identification of novel modulators and for the rational development of specific modulators of metabotropic glutamate receptors. The present invention also provides for polypeptides encoded by the herein disclosed isolated nucleic acid molecules that will find use in the identification of novel modulators and for the rational development of specific modulators of metabotropic glutamate receptors. The identification of the herein described nucleic acid and their respective mutant gene products will aid in the characterization and treatment of physiological disorders is hereby furthered.

In particular, the present invention advances the art by identifying at lease one site of allosteric modulation in mGluR2 and mGluR3, by characterizing the said site through such mutations. These mutations result in a dramatic effect on potentiation by modulators at said site. In particular, certain modulators that have a dramatic effect on wild type of mGluR2 are shown to have far less effect on variant mGluR2 receptors in which one or more such point mutations are introduced. Critically, such effect is not observed by the other specific subtype, mGluR3.

The present invention also provides for assays to identify and determine the efficacy and reaction profile of modulators acting at such newly identified allosteric binding sites, which proves useful in the treatment or prevention of disorders associated with an excess or deficiency in the amount of glutamate present. Modulators developed with a high specificity to a single type of metabotropic G-Protein Receptor is expected to have greater utility and efficicacy because: 1) as a modulator, it can function at lower concentrations than an agonist or antagonist; and 2) with great specificity, a modulator can precisely target the desired metabotropic G-Protein Receptor without affecting the activity of related receptors having different, potentially undesired cellular effects. This is perceived to provide for development of modulators whose use in a patient in need thereof results in less overall toxicity to said patient.

### BRIEF DESCRIPTION OF THE DRAWINGS

FIG. 1 provides a summary chart of the normalized response to glutamate in HEK-293 cells transfected with wild-type mGluR2 receptors, with some treatments exposed to two mGluR2 modulators, A and B.

FIG. 2 provides a summary chart of the normalized response to glutamate in HEK-293 cells transfected with a mutant mGluR2 receptor of the present invention, with some treatments exposed to two mGluR2 modulators, A and B.

FIG. 3 provides a summary chart of the normalized response to glutamate in HEK-293 cells transfected with another mutant mGluR2 receptor of the present invention, with some treatments exposed to two mGluR2 modulators, A and B.

FIG. 4 provides a summary chart of the normalized response to glutamate in HEK-293 cells transfected with another mutant mGluR2 receptor of the present invention, with some treatments exposed to two mGluR2 modulators, A and B.

FIG. 5 provides a summary chart of the normalized response to glutamate in HEK-293 cells transfected with another mutant mGluR2 receptor of the present invention, with some treatments exposed to two mGluR2 modulators, A and B.

FIG. 6 provides a summary chart of the normalized response to glutamate in HEK-293 cells transfected with another mutant mGluR2 receptor of the present invention, with some treatments exposed to two mGluR2 modulators, A and B.

### DETAILED DESCRIPTION OF THE INVENTION

Definitions and Terms of Art

The terms and abbreviations used in this document have their normal meanings unless otherwise designated. For example “° C.” refers to degrees Celsius; “N” refers to normal or normality; “mmol” refers to millimole or millimoles; “g” refers to gram or grams; “ml” means milliliter or milliliters; “M” refers to molar or molarity; “μg” and “ug” refer to microgram or micrograms; “μM” and “uM” refer to microMole or micromoles; and “μl” and “ul” refer to microliter or microliters. It is noted that some definitions are located outside this section.

All nucleic acid sequences, unless otherwise designated, are written in the direction from the 5′ end to the 3′ end, frequently referred to as “5′ to 3”′.

All amino acid or protein sequences, unless otherwise designated, are written commencing with the amino terminus (“N-terminus”) and concluding with the carboxy terminus (“C-terminus”).

“Base pair” or “bp” as used herein refers to DNA or RNA. The abbreviations A,C,G, and T correspond to the 5′-monophosphate forms of the deoxyribonucleosides (deoxy)adenosine, (deoxy)cytidine, (deoxy)guanosine, and (deoxy)thymidine, respectively, when they occur in DNA molecules. The abbreviations U,C,G, and A correspond to the 5′-monophosphate forms of the ribonucleosides urodine, cytidine, guanosine, and adenosine, respectively when they occur in RNA molecules. In double stranded DNA, base pair may refer to a pairing of A with T or C with G. In a DNA/RNA, heteroduplex base pair may refer to a pairing of A with U or C with G. (See the definition of “complementary”, infra.)

The terms “cleavage” or “restriction digestion” of DNA refers to the catalytic cleavage of the DNA with a restriction enzyme that acts only at certain sequences in the DNA (“sequence-specific endonucleases”). The various restriction enzymes used herein are commercially available and their reaction conditions, cofactors, and other requirements were used as would be known to one of ordinary skill in the art. Appropriate buffers and substrate amounts for particular restriction enzymes are specified by the manufacturer or can be readily found in the literature.

“Ligation” refers to the process of forming phosphodiester bonds between two nucleic acid fragments (T. Maniatis, et al., supra., p. 146). Unless otherwise provided, ligation may be accomplished using known buffers and conditions with a DNA ligase, such as T4 DNA ligase.

The term “plasmid” refers to an extrachromosomal (usually) self-replicating genetic element. Plasmids are generally designated by a lower case “p” followed by letters and/or numbers. The starting plasmids herein are either commercially available, publicly available on an unrestricted basis, or can be constructed from available plasmids in accordance with published procedures. In addition, equivalent plasmids to those described are known in the art and will be apparent to the ordinarily skilled artisan.

The term “reading frame” means the nucleotide sequence from which translation occurs “read” in triplets by the translational apparatus of transfer RNA (tRNA) and ribosomes and associated factors, each triplet corresponding to a particular amino acid. A frameshift mutation occurs when a base pair is inserted or deleted from a DNA segment. When this occurs, the result is a different protein from that coded for by the DNA segment prior to the frameshift mutation. To insure against this, the triplet codons corresponding to the desired polypeptide must be aligned in multiples of three from the initiation codon, i.e. the correct “reading frame” being maintained.

“Recombinant DNA cloning vector” as used herein refers to any autonomously replicating agent, including, but not limited to, plasmids and phages, comprising a DNA molecule to which one or more additional DNA segments can or have been added.

The term “recombinant DNA expression vector” as used herein refers to any recombinant DNA cloning vector in which a promoter and other regulatory elements to control transcription of the inserted DNA.

The term “expression vector system” as used herein refers to a recombinant DNA expression vector in combination with one or more trans-acting factors that specifically influence transcription, stability, or replication of the recombinant DNA expression vector. The transacting factor may be expressed from a co-transfected plasmid, virus, or other extrachromosomal element, or may be expressed from a gene integrated within the chromosome.

“Transcription” as used herein refers to the process whereby information contained in a nucleotide sequence of DNA is transferred to a complementary RNA sequence.

The term “transfection” as used herein refers to the taking up of an expression vector by a host cell whether or not any coding sequences are in fact expressed. Numerous methods of transfection are known to the ordinarily skilled artisan, for example, lipofectamine, calcium phosphate co-precipitation, and electroporation. Successful transfection is generally recognized when any indication of the operation of this vector occurs within the host cell.

The term “transformation” as used herein means the introduction of DNA into an organism so that the DNA is replicable, either as an extrachromosomal element or by chromosomal integration. Methods of transforming bacterial and eukaryotic hosts are well known in the art, many of which methods are summarized in J. Sambrook, et al., “Molecular Cloning: A Laboratory Manual” (1989).

The term “translation” as used herein refers to the process whereby the genetic information of messenger RNA is used to specify and direct the synthesis of a polypeptide chain.

The term “vector” as used herein refers to a nucleic acid compound used for the transformation of cells with polynucleotide sequences corresponding to appropriate protein molecules which when combined with appropriate control sequences confer specific properties on the host cell to be transformed. Plasmids, viruses, and bacteriophage are suitable vectors. Artificial vectors are constructed by joining DNA molecules from different sources. The term “vector” as used herein includes Recombinant DNA cloning vectors and Recombinant DNA expression vectors.

The terms “complementary” or “complementarity” as used herein refers to the pairing of bases, purines and pyrimidines, that associate through hydrogen bonding in double stranded nucleic acid. The following base pairs are complementary: guanine and cytosine; adenine and thymine; and adenine and uracil.

“Isolated amino acid sequence” refers to any amino acid sequence, however constructed or synthesized, which is locationally distinct from the naturally occurring sequence.

“Isolated DNA compound” refers to any DNA sequence, however constructed or synthesized, which is locationally distinct from its natural location in genomic DNA.

“Isolated nucleic acid compound” refers to any RNA or DNA sequence, however constructed or synthesized, which is locationally distinct from its natural location.

A “primer” is a nucleic acid fragment which functions as an initiating substrate for enzymatic or synthetic elongation.

The term “promoter” refers to a DNA sequence which directs transcription of DNA to RNA.

As used herein, a nucleic acid “probe” is single-stranded DNA or RNA, or analog thereof, that has a sequence of nucleotides that includes at least 14, preferably at least 20, more preferably at least 50, contiguous bases that are the same as or the complement of any 14 or more contiguous bases set forth in SEQ.ID.NO.: 1. In addition, the entire cDNA-encoding region of the entire sequence corresponding to SEQ.ID.NO.: 1 may be used as a probe.

Presently preferred probe-based screening conditions comprise a temperature of about 37° C., a formamide concentration of about 20%, and a salt concentration of about 5× standard saline citrate (SSC; 20×SSC contains 3M sodium chloride, 0.3M sodium citrate, pH 7.0). Such conditions will allow the identification of sequences which have a substantial degree of similarity with the probe sequence, without requiring perfect homology.

“Hybridization” refers to the binding of complementary strands of nucleic acid (i.e., sense:antisense strands or probe:target-DNA) to each other through hydrogen bonds, similar to the bonds that naturally occur in chromosomal DNA. Stringency levels used to hybridize a given probe with target-DNA can be readily varied by those of skill in the art.

The phrase “stringent hybridization conditioned” is used herein to refer to conditions under which polynucleic acid hybrids are stable. As known to those of skill in the art, the stability of hybrids is reflected in the melting temperature (Tₘ) of the hybrids. Tₘ can be approximated by the formula:
81.5° C.−16.6(log₁₀[Na⁺])+0.41(% G+C)−600/1,
where 1 is the length of the hybrids in nucleotides. Tₘ decreases approximately 1°-1.5° C. with every 1% decrease in sequence homology. In general, the stability of a hybrid is a function of sodium ion concentration and temperature. Typically, the hybridization reaction is performed under conditions of lower stringency, followed by washes of varying, but higher, stringency. Reference to hybridization stringency relates to such washing conditions.

As used herein, the phrase “moderately stringent hybridization” refers to conditions that permit target-DNA to bind a complementary nucleic acid that has about 60% identity, preferably about 75% identity, more preferably about 85% identity to the target DNA; with greater than about 90% identity to target-DNA being especially preferred. Preferably, moderately stringent conditions are conditions equivalent to hybridization in 50% formamide, 5× Denhart's solution, 5× SSPE, 0.2% SDS at 42° C., followed by washing in 0.2× SSPE, 0.2% SDS, at 65° C.

The phrase “high stringency hybridization” refers to conditions that permit hybridization of only those nucleic acid sequences that form stable hybrids in 0.01 8M NaCl at 65° C. (i.e., if a hybrid is not stable in 0.018M NaCl at 65° C., it will not be stable under high stringency conditions, as contemplated herein). High stringency conditions can be provided, for example, by hybridization in 50% formamide, 5× Denhart's solution, 5× SSPE, 0.2% SDS at 42° C., followed by washing in 0.1× SSPE, and 0.1% SDS at 65° C.

The phrase “low stringency hybridization” refers to conditions equivalent to hybridization in 10% formamide, 5× Denhart's solution, 6× SSPE, 0.2% SDS at 42° C., followed by washing in 1× SSPE, 0.2% SDS, at 50° C.

The term “antigenically distinct” as used herein refers to a situation in which antibodies raised against an epitope of the proteins of the present invention, or a fragment thereof, may be used to differentiate between the proteins of the present invention and other glutamate receptor subtypes. This term may also be employed in the sense that such antibodies may be used to differentiate between the mutant human mGluR2 and mGluR3 receptor proteins and analogous proteins derived from other species.

The term “PCR” as used herein refers to the widely known polymerase chain reaction employing a thermally-stable polymerase.

Further, as used herein, “modulator” may be any molecule, compound, or any other composition which is suspected of being capable of modulating the rate or other substantive characteristic of binding of glutamate to an mGluRx receptor in vivo or in vitro by acting at a site (an allosteric site) on the receptor that is not the agonist binding site. “Modulators” that are screened in the present invention can be any substances that are generally screened in the pharmaceutical industry during the drug development process. The substances may be macromolecules, such as biological polymers, including proteins, polysaccharides, nucleic acids, or the like. More usually, a substance will be a small molecule having a molecular weight below about 2 kD, more usually below 1.5 kD, frequently below 1 kD, and usually in the range from 100 to 1,000 D, and even more usually in the range from 200 D to 750 D. One or more substances may be pre-selected based on a variety of criteria. For example, suitable substances may be selected based upon SAR analysis based upon the calculated or predicted three-dimensional structures of the allosteric binding site discovered herein. Alternatively, the substances may be selected randomly and tested by the screening methods of the present invention. Substances which are able to up-modulate or down-modulate glutamate binding to an mGluRx receptor in vitro are considered as candidates for further screening of their ability to affect the activity of the tested mGluRx in cells and/or animals. Substances are often tested in the methods of the present invention as large collections of substances, e.g. libraries of low molecular weight organic compounds, peptides, or natural products.

In the scientific literature, the term “allosteric” has taken somewhat different meanings with different scopes (see “G Protein-Coupled Receptor Allosterism and Complexing” Pharmacological Reviews, 54:2, 323-374, at 326-327). As used herein, “allosteric” is taken to mean a site other than the ligand binding site where a non-ligand molecule (one that is neither an agonist nor an antagonist at the reactive binding site of the molecule) may bind, and wherein such binding affects the binding or the ligand at the ligand binding site (as expressed by rate, association constant, affinity, etc.). In contrast to some definitions in the field, as used herein the binding of a molecule at the ligand binding site need not affect the binding of a molecule at the allosteric site. An “allosteric modulator” is a modulator, as defined above, that binds to an allosteric site on a molecule of interest. It is noted that the same molecule may act as an allosteric modulator on one protein or receptor, and act as an agonist or antagonist with regard to a second protein or receptor.

With regard to the effect of a mutation of an mGluRx polypeptide as described herein, the terms “depotentiate(s)” and “depotentiation” are taken to mean that the effect of such mutation results in a measurable decrease in the effect of a modulator in combination with said mutant polypeptide in comparison to a non-mutated wild-type mGluRx of the same species and subtype. Preferably, the measurable decrease is substantial as that term is defined herein. Likewise, the terms “potentiate(s)” and “potentiation” are taken to mean that the effect of such mutation results in a measurable increase in the effect of a modulator in combination with said mutant polypeptide in comparison to a non-mutated wild-type mGluRx of the same species and subtype. Preferably, the measurable increase is substantial as that term is defined herein.

A “structure activity relationship” (SAR) refers to the relationship between a given chemical structure or series of chemical structures and the pharmacological activity that series of compounds has on the given target or action of the compound. Compounds can be classed together based on a number of characteristics including but not limited to such structural characteristics as shape, size, stereochemical arrangement, and distribution of functional groups. Other factors contributing to structure-activity relationships can include chemical reactivity, electronic effects, resonance, and inductive effects. The utilization of various assays that can differentiate between structural modifications that produce increased affinity or specificity of a given compound on its target compared to the activity seen on closely related targets or on targets that may be associated with off-target or unwanted activity of the compounds. The refinement of this SAR process can lead to pharmacologically active compounds that are more specific and less toxic than the initially identified compound or series of compounds.

A “conservative amino acid substitution” refers to the replacement of one amino acid residue by another, chemically similar, amino acid residue. Examples of such conservative substitutions are: substitution of one hydrophobic residue (isoleucine, leucine, valine, or methionine) for another; substitution of one polar residue for another polar residue of the same charge (e.g., arginine for lysine; glutamic acid for aspartic acid); substitution of one aromatic amino acid (tryptophan, tyrosine, or phenylalanine) for another.

A “conservative amino acid substitution” as defined above is but one type of variation of an amino acid sequence listing encompassed by the broader term, “conservatively modified variants thereof.” For instance, the latter is taken to have the meaning ascribed to the term in M.P.E.P. § 2422.03, Eighth Edition, 2001, which can include, without being limited to this example, deletions such as “at the C-terminus by 1, 2, 3, 4, or 5 residues.” Where appropriate within this specification, conservative amino acid substitutions that are known or reasonably predicted to not adversely alter the desired functionality of the novel sequences disclosed herein are disclosed Such disclosed conservative amino acid substitutions are considered to fall within the scope of the sequence listings that include the novel polypeptide sequences disclosed and claimed herein. The same principal of “conservatively modified variants” applies to nucleotide sequences as well, additionally taking into account the redundancy of codons for a particular amino acid, and the optimization of codons for expression in particular species.

For instance, but not meant to be limiting, an amino acid sequence or a nucleotide sequence is considered “identical” to a reference sequence if the two sequences are the same when aligned for maximum correspondence over a comparison window. Optimal alignment of nucleotide and amino acid sequences for aligning comparison window may be conducted by the local homology algorithm of Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Natl. Acad. Sci., U.S.A. 85:2444-2448, by computerized implementations of these algorithms (GAP, BESFIT, FASTA, and TFASTA in the Wisconsin Genetics Software Package Release 7.0, Genetics Computer Group, 575 Science Dr., Madison, Wis.), or by inspection. Such determination of identity can be considered to indicate a “conservatively modified variant” of a particular amino acid sequence or nucleotide sequence so long as the variant continues to function as a polypeptide or nucleic acid sequence according to the present invention.

“Consists essentially,” with respect to a mutant polypeptide, indicates that the reference sequence can be modified by N-terminal and/or C-terminal additions or deletions that do not cause a substantial decrease in the ability of the mutant polypeptide to function to affect the binding of an allosteric modulator at the site of the mutation compared to the reference sequence lacking such additions or deletions. An example of a deletion is the removal of an N-terminal methionine.

A “substantial change” in the ability of the mutant mGluR2 and mGluR3 proteins of the present invention to affect the modulation relative to a wild-type control, is defined to be a change of at least about 20%, more usually at least about 50%, preferably at least about 75%, and often at least about 90% or higher compared to the response of said wild-type control. This change may be a decrease or an increase relative to the wild-type control, e.g., a “substantial decrease” or a “substantial increase” per the definition of “substantial change” relative to such control.

The term “antibody” refers to a polypeptide substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes. Antibodies exist, e.g., as intact immunoglobulins or as a number of well-characterized fragments produced by digestion with various peptidases. These include, e.g., Fab′ and F(ab)′2 fragments. The term “antibody” also includes antibody fragments either produced by the modification of whole antibodies or those synthesized de novo using recombinant DNA methodologies, and further includes “humanized” antibodies made by conventional techniques.

The term “immunoassay” is an assay that utilizes an antibody to specifically bind an analyte. The immunoassay is characterized by the use of specific binding properties of a particular antibody to isolate, target, and/or quantify the analyte.

An antibody “specifically binds to” or “is specifically immunoreactive with” a protein, polypeptide, or peptide when the antibody functions in a binding reaction which is determinative of the presence of the protein, polypeptide, or peptide in the presence of a heterogeneous population of proteins and other biologics. Thus, under designated immunoassay conditions, the specified antibodies bind preferentially to a particular protein, polypeptide, or peptide and do not bind in a significant amount to other proteins, polypeptides, or peptides present in the sample. Specific binding to a protein, polypeptide, or peptide under such conditions requires an antibody that is selected for specificity for a particular protein, polypeptide, or peptide. As used herein, the term “recognize” as it regards an antibody's association to an a particular protein, polypeptide, or peptide, or an epitope therein, is taken to mean that said antibody “specifically binds to” or “is specifically immunoreactive with that protein, polypeptide, or peptide.”

A variety of immunoassay formats may be used to select antibodies specifically immunoreactive with a particular protein, polypeptide, or peptide. For example, solid-phase ELISA immunoassays are routinely used to select monoclonal antibodies specifically immunoreactive with a protein, polypeptide, or peptide. See Harlow & Lane, 1988, Antibodies, A Laboratory Manual, Cold Spring Harbor Publications, New York, for a description of immunoassay formats and conditions that can be used to determine specific immunoreactivity.

“Transfection” refers to any of the methods known in the art for introducing DNA into a cell, for example, but not limited to, the methods of lipofectamine, calcium phosphate or calcium chloride mediated transfection, electroporation, and infection with a retroviral vector.

Experiments Leading to Invention

The identification of the site of action of the mGluR2 potentiators was initiated based on the fact that compounds had been identified that positively modulated mGluR2 in the presence of EC₁₀ of glutamate but had no potentiator activity on mGluR3 (patent references, Lilly and Merck). The subtype specificity and selectivity of these compounds implied that a subset of the amino acid residues that differed between mGluR2 receptor and mGluR3 were most likely involved in the specific binding of the compound. Initial alignments of the amino acid sequences of human mGluR2 (hmGluR2) and human mGluR3 (hmGluR3) revealed an overall amino acid homology of 66.3%. In order to identify the site of action of the mGluR2 positive modulators, various regions that differed between the hmGluR2 and hmGluR3 receptors were identified and selected for chimeric substitution between sequences contained in hmGluR2 and hmGluR3.

The initial region selected for chimeric substitution was a region in the amino terminal domain of hmGluR2 that was 5′ of transmembrane one (Arginine-425 to Glutamic Acid-569). This domain contained a very cysteine rich region. It had recently been reported (Schweitzer et al. (2000)) that zinc selectively inhibited the agonist activity of glutamate on mGluR2 compared to mGluR3. This apparent subtype selectivity of the effect of zinc on mGluR2 compared to mGluR3 could be due to the presence of a zinc finger domain on mGluR2 that was not present on mGluR3. Schweitzer et al. (2000) had not identified the site of action of this zinc effect. We targeted the substitution of the cysteine rich region of mGluR2 for the homologous region in hmGluR3, since it has been widely reported that zinc binds to various cysteine and histidine motifs. Clearly the effect seen by the mGluR2 potentiators was the inverse effect seen by zinc however, it was thought that identification of the site of action of this zinc effect might lead to identification/understanding of the region involved in the potentiator effect.

After the construction of the cysteine-rich chimera was initiated, a series of subsequent chimeric molecules were made to various regions of the transmembrane domains of hmGluR2 and hmGluR3. The rational for these substitutions was based on the fact that many sites of action for agonists or antagonist have been localized on various subtypes of G-protein coupled receptors (GPCRs) to particular transmembrane domains of these receptors (insert references). However, to our knowledge, the site of action of a receptor specific potentiator had not been localized to a transmembrane domain. The site of action of a mGluR5-specific antagonist, MPEP, was recently localized to three specific residues contained in the metabotropic receptor subtype 5 cDNA, mGluR5a (insert reference) and since the metabotropic receptors represent a family of closely related GPCRs, it was thought that a transmembrane binding domain(s) could be involved in the binding of the mGluR2 potentiators. A series of chimeric constructs were subsequently generated that exchanged regions from TM1-3, TM3-5 or a single amino acid substitution in TM7 from hmGluR2 with homologous regions of hmGluR3.

### DETAILED DISCLOSURE OF INVENTION, PROTOCOLS, AND APPLICATIONS

First, it is noted that the disclosure herein, of mutant forms of mGluR2 and mGluR3, describes and specifies sequences for human forms of these mutant receptors. However, it is appreciated by those skilled in the art that the invention likewise applies to orthologues of such mutant forms in other species, including but not limited to murine species, in particular the rat and mouse, and also for monkey, ape, and other higher primates. Also, chimeric forms incorporating the novel sequences are prepared by combining genetic material from two or more species, for instance, combining a mutant form of human mGluR2 into a rat model genome.

In certain embodiments, the invention provides a mutant polypeptide, where the mutant polypeptide comprises substitution of at least one, two or three amino acids at positions 688, 689 and 735 relative to the wild-type human mGluR2 molecule, and specifically comprising any one, two in combination, or all three of the amino acid substitutions indicated below:

for mGluR2: leucine for serine at position 688; valine for glycine at position 689; and aspartic acid for asparagine at position 735.

for mGluR3: serine for leucine at position 688; glycine for valine at position 689; and asparagine for aspartic acid at position 735.

That is, based on the research described in the section above, it was learned that the substitutions of the amino acids in wild-type mGluR3 at homologous positions 688, 689 and 735, into mGluR2, substantially affected the potentiation of the resultant mutant mGluR2 molecules when said molecules were evaluated in the presence of an mGluR2 modulator that appears to bind to an allosteric binding site involving these amino acid positions. It is noted that said positions 688 and 689 are recognized to reside in transmembrane region 4 (TM4) of mGluR2, and said position 735 is recognized to reside in transmembrane region 5 (TM5) of mGluR2. While not being bound to a particular theory, it is hypothesized that, for mGluR2, the substitution of larger and/or more acidic amino acids at such positions results in a configurational change in an allosteric binding site situated in or near TM4 and TM5. It is further hypothesized that such configurational change makes binding by certain allosteric modulators at such site more difficult, or less effective, resulting in a depotentiation compared to a wild-type mGluR2.

Likewise, while not being bound to a particular theory, it is postulated that the “reverse” substitutions, of the wild-type mGluR2 amino acids at these positions in mGluR3, to form mGluR3 mutants, results in potentiation by allosteric modulators at an analogous site on the mutant mGluR3. Further, although this disclosure may state that modulators bind to an allosteric site associated with the amino acids at the above three positions, it is emphasized that this is merely based on a theory hypothesized from the evidence at hand. Critically, the novelty and the utility of the invention disclosed herein, particularly the mGluR2 and mGluR3 mutants and related methods, are not bound to this particular theory. Instead, the invention relies on the merits of the discoveries and results presented herein, independently of the ultimate accuracy of any of the above hypotheses and theories.

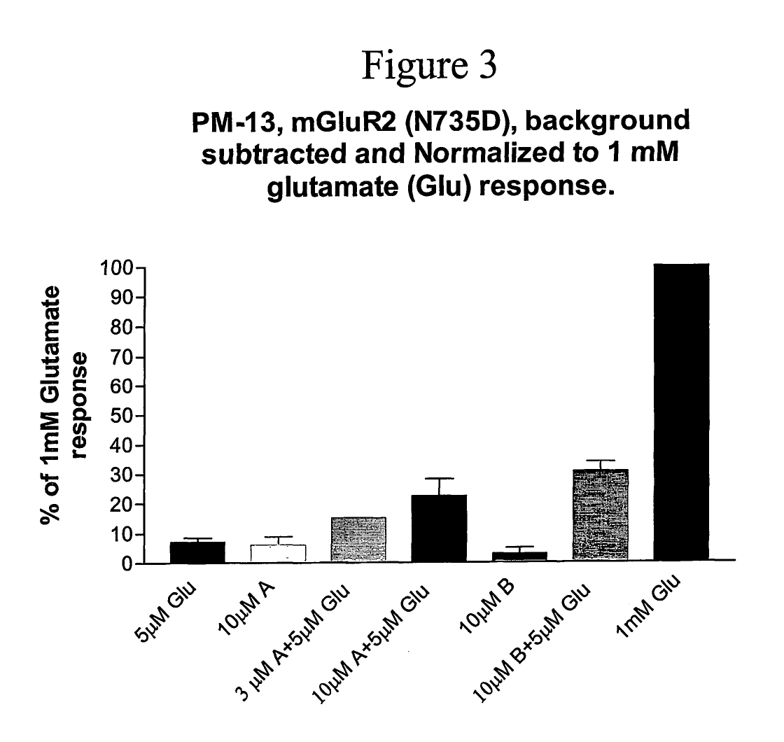

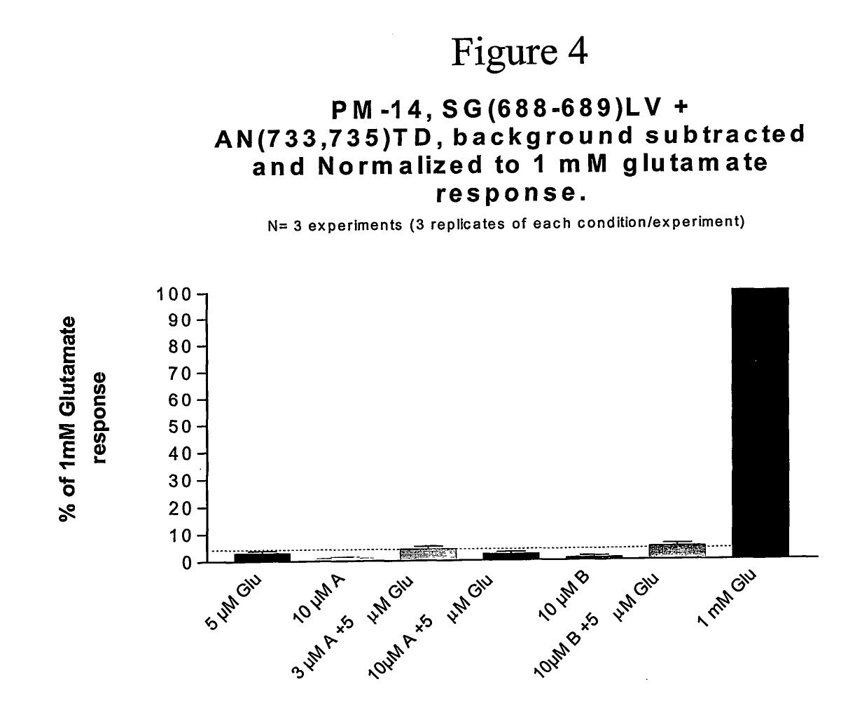

Particularly, this invention provides mutant mGluR2 polypeptide sequences selected from the group consisting of SEQ.ID.NOS.:1-8, and conservatively modified variants of such sequences. The data presented herein, particularly in FIG. 4 (see below), indicates that four single amino acid mutations together provide a substantial decrease in the effect of two allosteric modulators. However, it is believed that the three mutations noted above are the mutations with the greater effect relative to the fourth mutation, A733T. This belief is based on certain data (not shown) that appears to indicate that the A733T mutation (threonine replacing alanine at position 733) by itself provides an effect similar to that of a wild-type control. In contrast, the testing of the other single mutations by themselves (see FIG. 3 for N735D, other data not shown) indicates a greater difference from the wild-type control.

In other embodiments, the invention provides an isolated nucleic acid or a nucleic acid compound that comprises a nucleic acid sequence which encodes for a mutant polypeptide, where the mutant polypeptide comprises a substitution of any one, two in combination, or all three of the amino acid substitutions indicated below:

for mGluR2: leucine for serine at position 688; valine for glycine at position 689; and aspartic acid for asparagine at position 735.

for mGluR3: serine for leucine at position 688; glycine for valine at position 689; and asparagine for aspartic acid at position 735.

Particularly, this invention provides isolated nucleic acid sequences selected from the group consisting of SEQ.ID.NOS.:9-16, and conservatively modified variants of such sequences.

In the above description of the mutations of mGluR3, for convenience of this disclosure, the same numbering used for mGluR2 is used for the mGluR3. It is noted that there is an offset of nine amino acids in the numbering. For example, where a substitution at position 688 in mGluR3 is noted above, the actual position on the mGluR3 molecule is at position 697.

Skilled artisans will recognize that the polypeptide, peptides, and fusion proteins of the present invention can be synthesized by any number of different methods. The amino acid compounds of the invention can be made by chemical methods well known in the art, including solid phase peptide synthesis or recombinant methods. Both methods are described in U.S. Pat. No. 4,617,149, incorporated herein by reference.

The principles of solid phase chemical synthesis of polypeptides are well known in the art and may be found in general texts in the area. See. e.g., H. Dugas and C. Penney, Bioorganic Chemistry (1981) Springer-Verlag, New York, 54-92. For example, peptides may be synthesized by solid-phase methodology utilizing an Applied Biosystems 430A peptide synthesizer (commercially available from Applied Biosystems, Foster City Calif.) and synthesis cycles supplied by Applied Biosystems. Protected amino acids, such as t-butoxycarbonyl-protected amino acids, and other reagents are commercially available from many chemical supply houses.

Sequential t-butoxycarbonyl chemistry using double couple protocols are applied to the starting p-methyl benzhydryl amine resins for the production of C-terminal carboxamides. For the production of C-terminal acids, the corresponding pyridine-2-aldoxime methiodide resin is used. Asparagine, glutamine, and arginine are coupled using preformed hydroxy benzotriazole esters. The following side chain protection may be used:

Arg, Tosyl

Asp, cyclohexyl

Glu, cyclohexyl

Ser, Benzyl

Thr, Benzyl

Tyr, 4-bromo carbobenzoxy

Removal of the t-butoxycarbonyl moiety (deprotection) may be accomplished with trifluoroacetic acid (TFA) in methylene chloride. Following completion of the synthesis the peptides may be deprotected and cleaved from the resin with anhydrous hydrogen fluoride containing 10% meta-cresol. Cleavage of the side chain protecting group(s) and of the peptide from the resin is carried out at zero degrees Celcius or below, preferably −20° C. for thirty minutes followed by thirty minutes at 0° C.

After removal of the hydrogen fluoride, the peptide/resin is washed with ether, and the peptide extracted with glacial acetic acid and then lyophilized. Purification is accomplished by size-exclusion chromatography on a Sephadex G-10 (Pharmacia) column in 10% acetic acid.

The proteins of the present invention alternatively are produced by recombinant methods. Recombinant methods are preferred if a high yield is desired. A general method for the construction of any desired DNA sequence is provided in J. Brown, et al., Methods in Enzymology, 68:109 (1979). See also, J. Sambrook, et al., supra.

The basic steps in the recombinant production of desired proteins are:

a) construction of a natural, synthetic or semi-synthetic DNA encoding the protein of interest;

b) integrating said DNA into an expression vector in a manner suitable for the expression of the protein of interest, either alone or as a fusion protein;

c) transforming an appropriate eukaryotic or prokaryotic host cell with said expression vector,

d) culturing said transformed or transfected host cell in a manner to express the protein of interest; and

e) recovering and purfying the recombinantly produced protein of interest.

In general, prokaryotes are used for cloning of DNA sequences and constructing the vectors of the present invention. Prokaryotes may also be employed in the production of the protein of interest. For example, the Escherichia coli K12 strain 294 (ATCC No. 31446) is useful for the prokaryotic expression of foreign proteins. Other strains of E. coli (and their relevant genotypes) are well known and commonly used in the art.

Further, in addition strains of E. coli, bacilli such as Bacillus subtilis, other enterobacteriaceae such as Salmonella typhimurium or Serratia marcescans, and various Pseudomonas species may be used. In addition to these gram-negative bacteria, other bacteria, especially Streptomyces, spp., may be employed in the prokaryotic cloning and expression of the proteins of this invention.

Thus, suitable E. coli strains, as well as many other suitable prokaryote species and strains are known in the art, and are commercially available from suppliers such as: Bethesda Research Laboratories, Gaithersburg, Md. 20877 and Stratagene Cloning Systems, La Jolla, Calif. 92037; or are readily available to the public from sources such as the American Type Culture Collection, 12301 Parklawn Drive, Rockville, Md., 10852-1776. U.S. Pat. Nos. 6,017,697 and 6,387,655 disclose additional information regarding suitable species for use, and are incorporated by reference generally and for this specific purpose.

Except where otherwise noted or recognized in the art, the bacterial strains can be used interchangeably. Any particular type of bacterial host stated herein is not meant to limit the invention in any way. Genotype designations are in accordance with standard nomenclature. See, for example, J. Sambrook, et al., supra.

Promoters suitable for use with prokaryotic hosts include the β-lactamase [vector pGX2907 (ATCC 39344) contains the replicon and β-lactamase gene] and lactose promoter systems [Chang et al., Nature (London), 275:615 (1978); and Goeddel et al., Nature (London), 281:544 (1979)], alkaline phosphatase, the tryptophan (trp) promoter system [vector pATH1 (ATCC 37695) is designed to facilitate expression of an open reading frame as a trpE fusion protein under control of the trp promoter] and hybrid promoters such as the tac promoter (isolatable from plasmid pDR540 ATCC-37282). However, other functional bacterial promoters, whose nucleotide sequences are generally known, enable one of skill in the art to ligate them to DNA encoding the proteins of the instant invention using linkers or adapters to supply any required restriction sites. Promoters for use in bacterial systems also will contain a Shine-Dalgarno sequence operably linked to the DNA encoding the desired polypeptides. These examples are illustrative rather than limiting.

The proteins of this invention may be synthesized either by direct expression or as a fusion protein comprising the protein of interest as a translational fusion with another protein or peptide which may be removable by enzymatic or chemical cleavage. It is often observed in the production of certain peptides in recombinant systems that expression as a fusion protein prolongs the lifespan, increases the yield of the desired peptide, or provides a convenient means of purifying the protein of interest. A variety of peptidases (e.g. enterokinase and thrombin) which cleave a polypeptide at specific sites or digest the peptides from the amino or carboxy termini (e.g. diaminopeptidase) of the peptide chain are known. Furthermore, particular chemicals (e.g. cyanogen bromide) will cleave a polypeptide chain at specific sites. The skilled artisan will appreciate the modifications necessary to the amino acid sequence (and synthetic or semi-synthetic coding sequence if recombinant means are employed) to incorporate site-specific internal cleavage sites. See e.g., P. Carter, “Site Specific Proteolysis of Fusion Proteins”, Chapter 13 in Protein Purification: From Molecular Mechanisms to Large Scale Processes, American Chemical Society, Washington, D.C. (1990).

In addition to cloning and expressing the genes of interest in the prokaryotic systems discussed above, the proteins of the present invention may also be produced in eukaryotic systems. The present invention is not limited to use in a particular eukaryotic host cell. A variety of eukaryotic host cells are available from depositories such as the American Type Culture Collection (ATCC) and are suitable for use with the vectors of the present invention. The choice of a particular host cell depends to some extent on the particular expression vector used to drive expression of the human glutamate receptor-encoding nucleic acids of the present invention.

A preferred host cell line employed in this invention is the widely available cell line HEK293 (hereinafter referred to as “HEK293 ” or “293”). This cell line is available from the American Type Culture Collection under the accession number CRL-1573.

Cell lines, such as HEK293, produce glutamate endogenously. As a result, some amounts of glutamate are secreted into the culture medium, thereby making it somewhat difficult to express and study glutamate receptors in such cell lines due to the activation of the transfected receptor. However, measures can be taken to reduce the effects of this glutamate release on the functional characterization of the receptor. These include but are not limited to the use of neurotransport protein. For example, cell lines can be produced to express a plasmid in which the rat glutamate transporter gene (GLAST) is expressed. The glutamate levels in 24 hour medium of a cell line expressing GLAST will reduce the amount of glutamate in the medium to less than 3 micromolar, thus reducing the basal activation of the receptor and/or desensitation or the requirement for extensive washing to remove residual glutamate before assay procedures. See Storck, et al, Proc. Nat'l Acad. Sci. USA, 89:10955-59 (Nov. 1992) and Desai et al, Molecular Pharmacology, 48:648-657 (1995). Other measures also are helpful in dealing with the endogenous glutamate production. For instance, plating the cells on fibronectin coated plates and exchanging out the media 16 hours before the assay is performed are helpful in reducing and normalizing the effects of the release of glutamate to the media.

In addition to the lipid transfection method for incorporating the novel nucleic acid sequences (see Example 2 below), a wide variety of vectors, some of which are discussed below, exist for the transformation of mammalian host cells such as those described above. Thus, the present invention also relates to vectors which comprise a polynucleotide or polynucleotides of the present invention, and host cells which are genetically engineered with vectors of the invention and to the production of polypeptides of the invention by recombinant techniques. In another embodiment, the present invention relates to a recombinant DNA molecule comprising, 5′ to 3′, a promoter effective to initiate transcription in a host cell and the above-described invention nucleic acid molecule(s).

Cell-free translation systems can also be employed to produce such proteins using RNAs derived from the DNA constructs of the present invention.

Incorporation of cloned DNA into a suitable expression vector, transfection of eukaryotic cells with a plasmid vector or a combination of plasmid vectors, each encoding one or more distinct genes or with linear DNA, and selection of transfected cells are well known in the art (see, e.g., Sambrook et al. (1989) Molecular Cloning: A Laboratory Manual, Second Edition, Cold Spring Harbor Laboratory Press). Suitable means for introducing (transducing) expression vectors containing invention nucleic acid constructs into host cells to produce transduced recombinant cells (i.e., cells containing recombinant heterologous nucleic acid) are well known in the art (see, for review, Friedmann, 1989, Science, 244:1275-1281; Mulligan, 1993, Science, 260:926-932, each of which are incorporated herein by reference in their entirety).

As referred to above, exemplary methods of transduction include, e.g., infection employing viral vectors (see, e.g., U.S. Pat. Nos. 4,405,712 and 4,650,764), calcium phosphate transfection (U.S. Pat. Nos. 4,399,216 and 4,634,665), dextran sulfate transfection, electroporation, lipofection (see, e.g., U.S. Pat. Nos. 4,394,448 and 4,619,794), cytofection, particle bead bombardment, and the like. The heterologous nucleic acid can optionally include sequences which allow for its extrachromosomal (i.e., episomal) maintenance, or the heterologous nucleic acid can be donor nucleic acid that integrates into the genome of the host. Recombinant cells can then be cultured under conditions whereby the mutant mGluR2 and mGluR3 sequences encoded by the DNA is (are) expressed. Preferred cells include mammalian cells (e.g., HEK 293, CHO and Ltk-cells), yeast cells (e.g., methylotrophic yeast cells, such as Pichia pastoris), bacterial cells (e.g., Escherichia coli), and the like.

Expression vectors for use in carrying out the present invention will comprise a promoter capable of directing the transcription of a cloned DNA and a transcriptional terminator.

Also contained in the expression vectors is a polyadenylation signal located downstream of the coding sequence of interest. Polyadenylation signals include the early or late polyadenylation signals from SV40 (Kaufman and Sharp, ibid.), the polyadenylation signal from the Adenovirus 5 E1B region and the human growth hormone gene terminator (DeNoto et al., Nuc. Acid Res. 9: 3719-3730, 1981). The expression vectors may include a noncoding viral leader sequence, such as the Adenovirus 2 tripartite leader, located between the promoter and the RNA splice sites. Preferred vectors may also include enhancer sequences, such as the SV40 enhancer and the mouse μ enhancer (Gillies, Cell 33: 717-728, 1983). Expression vectors may also include sequences encoding the adenovirus VA RNAs.

Suitable expression vectors are well known in the art, and include vectors capable of expressing DNA operatively linked to a regulatory sequence, such as a promoter region that is capable of regulating expression of such DNA. Thus, an expression vector refers to a recombinant DNA or RNA construct, such as a plasmid, a phage, recombinant virus or other vector that, upon introduction into an appropriate host cell, results in expression of the inserted DNA. Appropriate expression vectors are well known to those of skill in the art and include those that are replicable in eukaryotic cells and/or prokaryotic cells and those that remain episomal or those which integrate into the host cell genome.

Exemplary expression vectors for transformation of E. coli prokaryotic cells include the pET expression vectors (Novagen, Madison, Wis., see U.S. Pat. No. 4,952,496), e.g., pET11a, which contains the T7 promoter, T7 terminator, the inducible E. coli lac operator, and the lac repressor gene; and pET 12a-c, which contains the 17 promoter, T7 terminator, and the E. coli ompT secretion signal. Another such vector is the pIN-IIIompA2 (see Duffaud et al., Meth. in Enzymology, 153:492-507, 1987), which contains the Ipp promoter, the lacUV5 promoter operator, the ompA secretion signal, and the lac repressor gene.

Exemplary eukaryotic expression vectors include eukaryotic cassettes, such as the pSV-2 gpt system (Mulligan et al., 1979, Nature, 277:108-114); the Okayama-Berg system (Mol. Cell Biol., 2:161-170), and the expression-cloning vector described by Genetics Institute (1985, Science, 228:810-815). Each of these plasmid vectors is capable of promoting expression of the invention chimeric protein of interest.

Also provided are nucleic acid molecule(s) comprising a transcriptional region functional in a cell, a sequence complimentary to an RNA sequence encoding an amino acid sequence corresponding to the herein-disclosed mutant mGluR2 and mGluR3 sequences, and a transcriptional termination region functional in a suitable host cell.

A wide variety of transcriptional and translational regulatory sequences may be employed, depending upon the nature of the host. The transcriptional and translational regulatory signals may be derived from viral sources, such as adenovirus, bovine papilloma virus, cytomegalovirus, simian virus, or the like, where the regulatory signals are associated with a particular gene sequence which has a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or are subject to chemical (such as metabolite) regulation. A favored promoter is the promoter from the Cytomegalovirus (CMV).

Thus, an embodiment provides are transformed host cells that recombinantly express the herein disclosed mutant mGluR2 and mGluR3 sequences of the invention.

As used herein, a cell is said to be “altered to express a desired peptide” when the cell, through genetic manipulation, is made to produce a protein which it normally does not produce or which the cell normally produces at lower levels. One skilled in the art can readily adapt procedures for introducing and expressing either genomic, cDNA, or synthetic sequences into either eukaryotic or prokaryotic cells.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene sequence expression. The precise nature of the regulatory regions needed for gene sequence expression may vary from organism to organism, but shall in general include a promoter region which, in prokaryotes, contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like.

If desired, the non-coding region 3′ to the sequence encoding an mGluR2 or mGluR3 gene may be obtained by the above-described methods. This region may be retained for its transcriptional termination regulatory sequences, such as termination and polyadenylation. Thus, by retaining the 3′-region naturally contiguous to the DNA sequence encoding a mutant mGluR2 and mGluR3 sequences, the transcriptional termination signals may be provided. Where the transcriptional termination signals are not satisfactorily functional in the expression host cell, then a 3′ region functional in the host cell may be substituted.

Two DNA sequencers (such as a promoter region sequence and an a mutant mGluR2 or mGluR3 encoding sequence) are said to be “operably linked” if the nature of the linkage between the two DNA sequences does not (1) result in the introduction of a frame-shift mutation, (2) interfere with the ability of the promoter region sequence to direct the transcription of the mutant mGluR2 or mGluR3 encoding gene sequence, or (3) interfere with the ability of the mutant mGluR2 or mGluR3 encoding sequence to be transcribed by the promoter region sequence. Thus, a promoter region would be operably linked to a DNA sequence if the promoter were capable of effecting transcription of that DNA sequence.

The selection of control sequences, expression vectors, transformation methods, and the like, are dependent on the type of host cell used to express the gene. As used herein, “cell”, “cell line”, and “cell culture” may be used interchangeably and all such designations include progeny.

The term “transformants” or “transformed cells” include the primary subject cell and cultures derived therefrom, without regard to the number of transfers. It is also understood that all progeny may not be precisely identical in DNA content, due to deliberate or inadvertent mutations. However, as defined, mutant progeny have the same functionality as that of the originally transformed cell.

To express the human mutant mGluR2 and mGluR3 sequences-encoding gene of the invention, transcriptional and translational signals recognized by an appropriate host are necessary. The present invention encompasses the expression of the mutant mGluR2 or mGluR3 sequences in either prokaryotic or eukaryotic cells.

Host cells which may be used in the expression systems of the present invention are not strictly limited, provided that they are suitable for use in the expression of the novel human mutant mGluR2 and mGluR3 sequences of the invention. Suitable hosts may often include eukaryotic cells, such as HEK-293 and other suitable mammalian cell lines.

Also, as noted above, representative examples of appropriate host cells for use in practicing the present invention include bacterial cells, such as streptococci, staphylococci, E. coli, Streptomyces and Bacillus subtilis cells. Also, fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells may be used.

Fungal cells, including species of yeast (e.g., Saccharomyces spp., particularly S. cerevisiae, Schizosaccharomyces spp.) or filamentous fungi (e.g., Aspergillus spp., Neurospora spp.) may be used as host cells within the present invention. Suitable yeast vectors for use in the present invention include YRp7 (Struhl et al., Proc. Natl. Acad. Sci. USA. 76: 1035-1039, 1978), YEp13 (Broach et al., Gene 8: 121-133, 1979), POT vectors (Kawasaki et al, U.S. Pat. No. 4,931,373, which is incorporated by reference herein), pJDB249 and pJDB219 (Beggs, Nature 275:104-108, 1978) and derivatives thereof Such vectors will generally include a selectable marker, which may be one of any number of genes that exhibit a dominant phenotype for which a phenotypic assay exists to enable transformants to be selected. Preferred selectable markers are those that complement host cell auxotrophy, provide antibiotic resistance or enable a cell to utilize specific carbon sources, and include LEU2 (Broach et al., ibid.), URA3 (Botstein et al., Gene 8: 17, 1979), HIS3 (Struhl et al., ibid.) or POT1 (Kawasaki et al., ibid.). Another suitable selectable marker is the CAT gene, which confers chloramphenicol resistance on yeast cells.

Any of a series of yeast gene sequence expression systems can be utilized which incorporate promoter and termination elements from the actively expressed gene sequences coding for glycolytic enzymes are produced in large quantities when yeast are grown in mediums rich in glucose. Known glycolytic gene sequences can also provide very efficient transcriptional control signals.

Yeast provides substantial advantages in that it can also carry out post-translational peptide modifications. A number of recombinant DNA strategies exist which utilize strong promoter sequences and high copy number of plasmids which can be utilized for production of the desired proteins in yeast. Yeast recognizes leader sequences on cloned mammalian gene sequence products and secretes peptides bearing leader sequences (i.e., pre-peptides). For a mammalian host, several possible vector systems are available for the expression of the mutant mGluR2 and mGluR3 sequences.

A variety of higher eukaryotic cells may serve as host cells for expression of the polypeptides of the invention, although not all cell lines will be capable of functional coupling of the receptor to the cell's second messenger systems. Cultured mammalian cells, such as BHK, CHO, Y1 (Shapiro et al., TIPS Suppl. 43-46 (1989)), NG108-15 (Dawson et al., Neuroscience Approached Through Cell Culture, Vol. 2, pages 89-114 (1989)), N1E-115 (Liles et al., J. Biol. Chem. 261:5307-5313 (1986)), PC 12 and COS-1 (ATCC CRL 1650) are preferred. Preferred BHK cell lines are the tk.sup.—ts13 BHK cell line (Waechter and Baserga, Proc. Natl. Acad. Sci. USA 79:1106-1110 (1982)) and the BHK 570 cell line (deposited with the American Type Culture Collection, 12301 Parklawn Dr., Rockville, Md. under accession number CRL 10314). A tk.sup.—BHK cell line is available from the ATCC under accession number CRL 1632.

Prokaryotic hosts are, generally, very efficient and convenient for the production of recombinant proteins and are, therefore, one type of preferred expression system for the expressing the mutant mGluR2 and mGluR3 sequences encoding gene.

Prokaryotes most frequently are represented by various strains of E. coli. However, other microbial strains may also be used, including other bacterial strains. In prokaryotic systems, plasmid vectors that contain replication sites and control sequences derived from a species compatible with the host may be used. Examples of suitable plasmid vectors may include pBR322, pUC-118, pUC 119 and the like; suitable phage or bacteriophage vectors may include .gamma.gt10, .gamma.gt11 and the like; and suitable virus vectors may include pMAM-neo, pKRC and the like. Preferably, the selected vector of the present invention has the capacity to replicate in the selected host cell.

Recognized prokaryotic hosts include bacteria such as E. coli, Bacillus, Streptomyces, Pseudomonas, Salmonella, Serratia, and the like. However, under such conditions, the peptide will not be glycosylated. The prokaryotic host must be compatible with the replicon and control sequences in the expression plasmid.

To express the mutant mGluR2 and mGluR3 sequences (or a functional derivative thereof) in a prokaryotic cell, it is necessary to operably link the mutant mGluR2 or mGluR3 encoding nucleotide sequence to a functional prokaryotic promoter. Such promoters may be either constitutive or, more preferably, regulatable (i.e., inducible or derepressible). Examples of constitutive promoters and inducible promoters of well known to a skilled artisan. Prokaryotic promoters are reviewed by Cenatiempo (Biochimie 68:505-516 (1986)); and Gottesman (Ann. Rev. Genet. 18:415-442 (1984) ). Proper expression in a prokaryotic cell also requires the presence of a ribosome-binding site upstream of the gene sequence-encoding sequence. Such ribosome binding sites are disclosed, for example, by Gold et at., Ann. Rev. Microbiol. 35:365-404 (1981).

As used herein, the term “promoter” refers to a polynucleotide sequence, preferably a DNA sequence, that regulates expression of a selected DNA sequence operably linked to the promoter, and which effects expression of the selected DNA sequence in cells. The term encompasses “tissue specific” promoters, i.e. promoters, which effect expression of the selected DNA sequence only in specific cells (e.g. cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e. expression levels can be controlled).

A mutant mGluR2 and mGluR3 sequences encoding nucleic acid molecule and an operably linked promoter may be introduced into a recipient prokaryotic or eukaryotic cell either as a nonreplicating DNA (or RNA) molecule, which may either be a linear molecule or, more preferably, a closed covalent circular molecule. Since such molecules are incapable of autonomous replication, the expression of the gene may occur through the transient expression of the introduced sequence; Alternatively, permanent expression may occur through the integration of the introduced DNA sequence into the host chromosome.

In one embodiment, a vector is employed which is capable of integrating the desired gene sequences into the host cell chromosome. Cells which have stably integrated the introduced DNA into their chromosomes can be selected by also introducing one or more markers which allow for selection of host cells which contain the expression vector. The marker may provide for prototrophy to an auxotrophic host, biocide resistance, e.g., antibiotics, or heavy metals, such as copper, or the like. The selectable marker gene sequence can either be directly linked to the DNA gene sequences to be expressed, or introduced into the same cell by co-transfection. Additional elements may also be needed for optimal synthesis of single chain binding protein mRNA. These elements may include splice signals, as well as transcription promoters, enhancers, and termination signals. cDNA expression vectors incorporating such elements include those described by Okayama, Molec. Cell. Biol. 3:280 (1983).

In a preferred embodiment, the introduced nucleic acid molecule will be incorporated into a plasmid or viral vector capable of autonomous replication in the recipient host. Any of a wide variety of vectors may be employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Preferred prokaryotic vectors include plasmids such as those capable of replication in E. coli (such as, for example, pBR322, ColE1, pSC101, pACYC 184, .pi.VX. Such plasmids are, for example, disclosed by Sambrook (cf. Molecular Cloning: A Laboratory Manual, second edition, edited by Sambrook, Fritsch, & Maniatis, Cold Spring Harbor Laboratory, (1989)). Bacillus plasmids include pC194, pC221, pT127, and the like. Such plasmids are disclosed by Gryczan (In: The Molecular Biology of the Bacitli, Academic Press, N.Y. (1982), pp. 307-329). Suitable Streptomyces plasmids include p1J101 (Kendall et al., J. Bacteriol. 169:4177-4183 (1987)), and streptomyces bacteriophages such as .phi.C31 (Chater et al., In: Sixth International Symposium on Actinomycetales Biology, Akademiai Kaido, Budapest, Hungary (1986), pp. 45-54). Pseudomonas plasmids are reviewed by John et al. (Rev. Infect. Dis. 8:693-704 (1986)), and Izaki (Jpn. J. Bacteriol. 33:729-742 (1978)).

As noted, supra, expression of the mutant mGluR2 and mGluR3 sequences in eukaryotic hosts requires the use of eukaryotic regulatory regions. Such regions will, in general, include a promoter region sufficient to direct the initiation of RNA synthesis. Preferred eukaryotic promoters include, for example, the Cytomegalovirus Promoter (CMV), the promoter of the mouse metallothionein I gene sequence (Hamer et al., J. Mol. Appl. Gen. 1:273-288 (1982)); the TK promoter of Herpes virus (McKnight, Cell 31:355-365 (1982)); the SV40 early promoter (Benoist et al., Nature (London) 290:304-310(1981)); the yeast gal4 gene sequence promoter (Johnston et al., Proc. Natl. Acad. Sci. (USA) 79:6971-7975 (1982); Silver et al., Proc. Natl. Acad. Sci. (USA) 81:5951-5955 (1984)).

As is widely known, translation of eukaryotic mRNA is initiated at the colon which encodes the first methionine. For this reason, it is preferable to ensure that the linkage between a eukaryotic promoter and a DNA sequence which encodes the mutant mGluR2 and mGluR3 sequences (or a finctional derivative thereof) does not contain any intervening codons which are capable of encoding a methionine (i.e., AUG). The presence of such codons results either in a formation of a fusion protein (if the AUG codon is in the same reading frame as the mutant mGluR2 or mGluR3 coding sequence) or a frame-shift mutation (if the AUG codon is not in the same reading frame as the mutant mGluR2 or mGluR3 coding sequence).

Preferred eukaryotic plasmids include, for example, BPV, vaccinia, SV40, 2-micron circle, and the like, or their derivatives. Such plasmids are well known in the art (Botstein et al., Miami Wntr. Symp. 19:265-274 (1982); Broach, In: The Molecular Biology of the Yeast Saccharomyces: Life Cycle and Inheritance, Cold Spring Harbor Laboratory, Cold Spring Harbor, N.Y., p. 445-470 (1981); Broach, Cell 28:203-204 (1982); Bollon et at., J. Clin. Hematol. Oncol. 10:39-48 (1980); Maniatis, In: Cell Biology: A Comprehensive Treatise, Vol. 3, Gene Sequence Expression, Academic Press, N.Y., pp. 563-608 (1980).

Once the vector or nucleic acid molecule containing the construct(s) has been prepared for expression, the DNA construct(s) may be introduced into an appropriate host cell by any of a variety of suitable means, i.e., transformation, transfection, conjugation, protoplast fusion, electroporation, particle gun technology, calcium phosphate-precipitation, direct microinjection, and the like. After the introduction of the vector, recipient cells are grown in a selective medium, which selects for the growth of vector-containing cells. Expression of the cloned gene molecule(s) results in the production of the herein-disclosed mutant mGluR2 and mGluR3 sequences or biologically active fragments thereof. This can take place in the transformed cells as such, or following the induction of these cells to differentiate (for example, by administration of bromodeoxyuracil to neuroblastoma cells or the like).

A variety of incubation conditions can be used to form the peptide of the present invention. Preferred conditions include those which mimic physiological conditions.

An example of the means for preparing the mutant mGluR2 and mGluR3 sequences of the invention is to express nucleic acids encoding the mutant mGluR2 and mGluR3 sequences in a suitable host cell, such as a bacterial cell, a yeast cell, an amphibian cell (i.e., oocyte), or a mammalian cell, using methods well known in the art, and recovering the expressed polypeptide, again using well-known methods.

Using methods such as northern blot or slot blot analysis, transfected cells that contain mutant mGluR2 and mGluR3 sequences encoding DNA or RNA can be selected. Transfected cells can also be analyzed to identify those that express the mutant mGluR2 and mGluR3 sequences. Analysis can be carried out, for example, by using any of well known screening assays attending a functional receptor, and comparing the values obtained to a control, untransfected host cells by electrophysiologically monitoring the currents through the cell membrane in response to mutant mGluR2 and mGluR3 sequences, and the like. Mutant mGluR2 and mGluR3 sequences(s) can be isolated directly from cells that have been transformed with expression vectors comprising nucleic acid encoding the mutant mGluR2 and mGluR3 sequences or fragments/portions thereof.

Nucleic acid molecules may be stably incorporated into cells or may be transiently introduced using methods known in the art. Stably transfected mammalian cells may be prepared by transfecting cells with an expression vector comprising a sequence of nucleotides that encodes the mutant mGluR2 and mGluR3 sequences in conjunction with a selectable marker gene (such as, for example, the gene for thymidine kinase, dihydrofolate reductase, neomycin resistance, and the like), and growing the transfected cells under conditions selective for cells expressing the marker gene. To prepare transient transfectants, mammalian cells are transfected with a reporter gene (such as the E. coli β-galactosidase gene) to monitor transfection efficiency. The precise amounts and ratios of DNA encoding the mutant mGluR2 and mGluR3 sequences may be empirically determined and optimized for a particular cells and assay conditions. Selectable marker genes are typically not included in the transient transfections because the transfectants are typically not grown under selective conditions, and are usually analyzed within a few days after transfection.

In order to identify cells that have integrated the cloned DNA, a selectable marker is generally introduced into the cells along with the gene or cDNA of interest. Preferred selectable markers for use in cultured mammalian cells include genes that confer resistance to drugs, such as neomycin, hygromycin, and methotrexate. The selectable marker may be an amplifiable selectable marker. Preferred ampliflable selectable markers are the DHFR gene and the neomycin resistance gene. Selectable markers are reviewed by Thilly (Manunalian Cell Technology, Butterworth Publishers, Stoneham, Mass., which is incorporated herein by reference). The choice of selectable markers is well within the level of ordinary skill in the art.

Selectable markers may be introduced into the cell on a separate plasmid at the same time as the gene of interest, or they may be introduced on the same plasmid. If on the same plasmid, the selectable marker and the gene of interest may be under the control of different promoters or the same promoter, the latter arrangement producing a dicistronic message. Constructs of this type are known in the art (for example, Levinson and Simonsen, U.S. Pat. No. 4,713,339). It may also be advantageous to add additional DNA, known as “carrier DNA” to the mixture which is introduced into the cells.

In particularly preferred aspects, eukaryotic cells which contain heterologous DNAs express such DNA and form recombinant mutant mGluR2 and mGluR3 sequences. In more preferred aspects, recombinant mutant mGluR2 and mGluR3 sequences activity is readily detectable because it is a type that is absent from the untransfected host cell.

Heterologous DNA may be maintained in the cell as an episomal element or may be integrated into chromosomal DNA of the cell. The resulting recombinant cells may then be cultured or subcultured (or passaged, in the case of mammalian cells) from such a culture or a subculture thereof. Methods for transfection, injection and culturing recombinant cells are known to the skilled artisan. Similarly, the mutant mGluR2 and mGluR3 sequences(s) may be purified using protein purification methods known to those of skill in the art. For example, antibodies or other ligands that specifically bind to the mutant mGluR2 and mGluR3 sequences may be used for affinity purification of the mutant mGluR2 and mGluR3 sequences.

As used herein, “heterologous or foreign DNA and/or RNA” are used interchangeably and refer to DNA or RNA that does not occur naturally as part of the genome of the cell in which it is present or to DNA or RNA which is found in a location or locations in the genome that differ from that in which it occurs in nature. Typically, heterologous or foreign DNA and RNA refer to DNA or RNA that is not endogenous to the host cell and has been artificially introduced into the cell. Examples of heterologous DNA include DNA disclosed herein.

In other embodiments, mRNA may be produced by in vitro transcription of DNA encoding the mutant mGluR2 and mGluR3 sequences. This mRNA can then be injected into Xenopus oocytes where the RNA directs the synthesis of the mutant mGluR2 and mGluR3 sequences. Alternatively, the invention-encoding DNA can be directly injected into oocytes for expression of a functional mutant mGluR2 and mGluR3 sequences. The transfected mammalian cells or injected oocytes may then be used in the methods of drug screening provided herein.

Alternatively, the invention DNA sequences can be transcribed into RNA, which can then be transfected into amphibian cells for translation into protein. Suitable amphibian cells include Xenopus oocytes.

Practitioners of this invention realize that, in addition to the above-mentioned expression systems, the cloned cDNA may also be employed in the production of transgenic animals in which a test mammal, usually a mouse, in which the effects of the expression of the proteins of the present invention can be assessed, particularly with regard to the interaction of such proteins and modulators or portions of modulators. For instance, without being limited by such example, the nucleic acids of the present invention may be employed in the construction of “knockin” animals in which the expression of a mutant mGluR2 or mGluR3 is expressed. Once such test animals are prepared, evaluations are conducted that evaluate the effects of modulator compounds. For instance, a modulator development program, based on rational drug design using SAR, may have a putative highly selective modulator design. Structural variations of this design, and the hypothetical optimal design, each are evaluated in a number of different types of knock-in mice, where each knock-in mouse type carries a different mutation combination of the S688L, G689V and N735D mutants. This data is used to verify, refine the design of, and evaluate the putative highly selective modulator in a whole organism model. An important feature of such evaluations is the ability to evaluate and assess “off-target” effects of the modulators, i.e., effects at other organs, or unexpected behavioral effects.

Skilled artisans also recognize that some alterations of SEQ.ID.NOS.:1-8 will fail to change the function of the respective amino acid compound. For instance, some hydrophobic amino acids may be exchanged for other hydrophobic amino acids. Those altered amino acid compounds which confer substantially the same function in substantially the same manner as the exemplified amino acid compound are also encompassed within the present invention. Typically such conservative substitutions attempt to preserve the: (a) secondary or tertiary structure of the polypeptide backbone; (b) the charge or hydrophobicity of the residue; or (c) the bulk of the side chain. Some examples of such conservative substitutions of amino acids, resulting in the production of proteins which may be functional equivalents of the protein of SEQ.ID.NOS.:1-8 are shown in TABLE II, infra.

TABLE II

| Original Residue | Exemplary Substitutions |
|---|---|
| Ala | Ser, Gly |
| Arg | Lys |
| Asn | Gln, His |
| Asp | Glu |
| Cys | Ser |
| Gln | Asn |
| Glu | Asp |
| Gly | Pro, Ala |
| His | Asn, Gln |
| lle | Leu, Val |
| Leu | Ile, Val |
| Lys | Arg, Gln, Glu |
| Mel | Leu, Ile |
| Phe | Met, Leu, Gyr |
| Ser | Thr |
| Thr | Ser |
| Trp | Tyr |
| Tyr | Trp, Phe |
| Val | Ile, Leu |

These substitutions may be introduced into the protein in a variety of ways, such as during the chemical synthesis or by chemical modification of an amino acid side chain after the protein has been prepared.

Alterations of the protein having a sequence which corresponds to the sequence of SEQ.ID.NOS.:1-8 also may be induced by alterations of the nucleic acid compounds which encode these proteins, i.e., SEQ.ID.NOS.:9-16. These mutations of the nucleic acid compound may be generated by either random mutagenesis techniques, such as those techniques employing chemical mutagens, or by site-specific mutagenesis employing oligonucleotides. Those nucleic acid compounds which confer substantially the same function in substantially the same manner as the exemplified nucleic acid compounds are also encompassed within the present invention.

Other embodiments of the present invention are nucleic acid compounds which comprise isolated nucleic acid sequences which encode any of the polypeptide sequences of SEQ.ID.NOS.:1-8. As skilled artisans will recognize, the amino acid compounds of the invention can be encoded by a multitude of different nucleic acid sequences because most of the amino acids are encoded by more than one nucleic acid triplet due to the degeneracy of the amino acid code. Because these alternative nucleic acid sequences would encode the same amino acid sequences, the present invention further comprises these alternate nucleic acid sequences.

The nucleic acid sequences encoding the mutant mGluRx receptor molecules of the present invention may be produced using synthetic methodology. This synthesis of nucleic acids is well known in the art. See. e.g., E. L. Brown, R. Belagaje, M. J. Ryan, and H. G. Khorana, Methods in Enzymology, 68:109-151 (1979). The DNA segments corresponding to the receptor gene are generated using conventional DNA synthesizing apparatus such as the Applied Biosystems Model 380A or 380B DNA synthesizers (commercially available from Applied Biosystems, Inc., 850 Lincoln Center Drive, Foster City, Calif. 94404) which employ phosphoramidite chemistry. In the alternative, the more traditional phosphotriester chemistry may be employed to synthesize the nucleic acids of this invention. [(See. e.g., M. J. Gait, ed., Oligonucleotide Synthesis, A Practical Approach, (1984).]

Any of such synthetic human mGluRx nucleic acid sequences (e.g., synthetic genes) may be designed to possess restriction endonuclease cleavage sites at either end of the transcript to facilitate isolation from and integration into expression and amplification plasmids. The choice of restriction sites are chosen so as to properly orient the coding sequence of the receptor with control sequences to achieve proper in-frame reading and expression of the human mGluRx receptor molecule. A variety of other such cleavage sites may be incorporated depending on the particular plasmid constructs employed and may be generated by techniques well known in the art.

In an alternative methodology, the desired DNA sequences can be generated using the polymerase chain reaction as described in U.S. Pat. No. 4,889,818, which is incorporated herein by reference.

In addition to the deoxyribonucleic acid of SEQ.ID.NOS.:9-16, this invention also provides ribonucleic acids (RNA) that likewise express the mGluR2 mutants designated as SEQ.ID.NOS.:1-8, and conservatively modified variants thereof. As is known to those of skill in the art, the DNA sequences provided herein are convertible to corresponding RNA sequences via pyrimidine base substitution of uracil (U) for thymine (T).

The ribonucleic acids of the present invention may be prepared using the polynucleotide synthetic methods discussed supra or they may be prepared enzymatically using RNA polymerases to transcribe a DNA template.

One system for preparing the ribonucleic acids of the present invention employs the RNA polymerase from the bacteriophage T7 or the bacteriophage SP6. Both of these RNA polymerases are highly specific and require the insertion of bacteriophage-specific sequences at the 5′ end of the message to be read. See, J. Sambrook, et al., supra, at 18.82-18.84.

This invention also provides nucleic acids, RNA or DNA, which are complementary to SEQ.ID.NOS.:9-16.

The present invention also provides probes and primers useful for molecular biology techniques. A compound which encodes for SEQ.ID.NOS.:9-16, or a complementary sequence of SEQ.ID.NOS.: 9-16, or a fragment thereof, and which is at least 20-50 base pairs in length, and which will selectively hybridize to human genomic DNA or messenger RNA encoding a human glutamate receptor, is provided. Preferably, the 25 or more base pair compound is DNA or a length sufficient to hybridize.

The term “selectively hybridize” as used herein may refer to either of two situations. In the first such embodiment of this invention, the nucleic acid compounds described supra hybridize to a human glutamate receptor under more stringent hybridization conditions than these same nucleic acid compounds would hybridize to an analogous glutamate receptor of another species, e.g. rodent. In the second such embodiment of this invention, these probes hybridize to the mutant mGluR2 or mGluR3 receptor molecules under more stringent hybridization conditions than other related compounds, including nucleic acid sequences encoding other glutamate receptors of the same species. A more detailed explanation of hybridization and varying stringency of hybridization, which pertain to the two types of hybridization described here, is provided in the “Definitions and Terms of Art” section supra.

These probes and primers can be prepared enzymatically as described supra. These probes and primers also are synthesized using chemical means as described supra. Alternately, these probes and primers of defined structure may also be purchased commercially.

Another aspect of the present invention is recombinant DNA cloning vectors and expression vectors comprising the nucleic acid sequences of the present invention. Many of the vectors encompassed within this invention are described above. The preferred nucleic acid vectors are those which are DNA. A preferred recombinant DNA vector comprises the isolated DNA sequence SEQ.ID.NO.:14, bearing the codons for all three single amino acid substitutions on the respective mutant mGluR2 sequence. This sequence has been shown to provide the strongest depotentiation. However, it is fully appreciated that when using the compositions of the present invention to help identify desired structures and sequences for effective modulators of mGluR2, the isolated nucleic acid sequences encoding a mutant mGluR2 with a single amino acid substitution, e.g., SEQ.ID.NO.:8-10, and the sequences encoding a mutant mGluR2 with two amino acid substitutions, e.g., SEQ.ID.NO.:11-13, are valuable when used in concert. Any of these are suitable for transfection into cells by use of vectors, as described herein, and by other means of transfection, such as by lipid transfection as in Example 2 below.

The skilled artisan understands that the type of cloning vector or expression vector employed depends upon the availability of appropriate restriction sites, the type of host cell in which the vector is to be transfected or transformed, the purpose of the transfection or transformation (e.g., transient expression in an oocyte system, stable transformation as an extrachromosomal element, or integration into the host chromosome), the presence or absence of readily assayable markers (e.g., antibiotic resistance markers, metabolic markers, or the like), and the number of copies of the gene to be present in the cell.

The type of vector employed to carry the nucleic acids of the present invention may be RNA viruses, DNA viruses, lytic bacteriophages, lysogenic bacteriophages, stable bacteriophages, plasmids, viroids, and the like. The most preferred vectors of the present invention are those derived from plasmids.

When preparing an expression vector the skilled artisan understands that there are many variables to be considered. One such example is the use of a constitutive promoter, i.e. a promoter which is functional at all times, instead of a regulatable promoter which may be activated or inactivated by the artisan using heat, addition or removal of a nutrient, addition of an antibiotic, and the like. The practitioner also understands that the amount of nucleic acid or protein to be produced dictates, in part, the selection of the expression system. For experiments examining the amount of the protein expressed on the cell membrane or for experiments examining the biological function of an expressed membrane protein, for example, it may be unwise to employ an expression system which produces too much of the protein. The addition or subtraction of certain sequences, such as a signal sequence preceding the coding sequence, may be employed by the practitioner to influence localization of the resulting polypeptide. Such sequences added to or removed from the nucleic acid compounds of the present invention are encompassed within this invention.

The plasmid pcDNA3.1 can be readily modified to construct expression vectors that produce mutant mGluR2 and mGluR3 receptors of the present invention in a variety of cells, including, for example, HEK-293.

One of the most widely employed techniques for altering a nucleic acid sequence is by way of oligonucleotide-directed site-specific mutagenesis. B. Comack, “Current Protocols in Molecular Biology”, 8.01-8.5.9, (F. Ausubel, et al., eds. 1991). In this technique an oligonucleotide, whose sequence contains the mutation of interest, is synthesized as described supra. This oligonucleotide is then hybridized to a template containing the wild-type sequence. In a most preferred embodiment of this technique, the template is a single-stranded template. Particularly preferred are plasmids which contain regions such as the f1 intergenic region. This region allows the generation of single-stranded templates when a helper phage is added to the culture harboring the “phagemid”.

After the annealing of the oligonucleotide to the template, a DNA-dependent DNA polymerase is then used to synthesize the second strand from the oliognucleotide, complementary to the template DNA. The resulting product is a heteroduplex molecule containing a mismatch due to the mutation in the oligonucleotide. After DNA replication by the host cell a mixture of two types of plasmid are present, the wild-type and the newly constructed mutant. This technique permits the introduction of convenient restriction sites such that the coding sequence may be placed immediately adjacent to whichever transcriptional or translational regulatory elements are employed by the practitioner.

The construction protocols utilized for E. coli can be followed to construct analogous vectors for other organisms, merely by substituting, if necessary, the appropriate regulatory elements using techniques well known to skilled artisans. Also, construction protocols for eukaryotic cells are widely known and employed by those skilled in the art.

Host cells which harbor the nucleic acids provided by the present invention are also provided. A preferred host cell is an Xenopus sp. oocyte which has been injected with RNA or DNA compounds of the present invention. Most preferred oocytes of the present invention are those which harbor a sense mRNA of the present invention. Other preferred host cells include HEK-293 cells which have been transfected and/or transformed with a vector which comprises a nucleic acid of the present invention.

The present invention also provides a method for constructing a recombinant host cell capable of expressing any of SEQ.ID.NOS.:9-16, said method comprising transforming a host cell with a recombinant DNA vector that comprises an isolated DNA sequence which encodes any of SEQ.ID.NOS.:9-16. The preferred host cell is HEK-293. A preferred vector for expression is one which comprises SEQ.ID.NO.:16. A preferred host cell for this method is HEK-293. An especially preferred expression vector in HEK-293 is one which comprises SEQ.ID.NO.:16.

Transformed host cells may be cultured under conditions well known to skilled artisans such that any of SEQ.ID.NOS.:9-16 is expressed, thereby producing a mutant of mGluR2 in the recombinant host cell, where such mutant alters the potentiation compared to wild type mGluR2 with respect to modulators that normally bind to at least one allosteric binding site associated with the three amino acid substitutions believed to provide the most substantial alteration in effect.

Likewise, transformed host cells may be cultured under conditions well known to skilled artisans such that any of SEQ.ID.NOS.:41-48 is expressed, thereby producing a mutant of mGluR3 in the recombinant host cell, where such mutant alters the potentiation compared to wild type mGluR3 with respect to modulators that normally bind to at least one allosteric binding site associated with the three amino acid substitutions believed to provide the most substantial alteration in effect. Also with regard to mGluR3, mutant peptides of SEQ.ID.NOS.:33-40 are produced and used in manners disclosed herein for mutant mGluR2 peptides. With regard to the use of such mGluR3 mutant peptides and the nucleic acid sequences encoding them, it is believed that the polypeptide configuration of at least some of such mutants interacts with an allosteric modulator having its site in the general TM4/TM5 region in such a way as to increase the effect of such modulator.

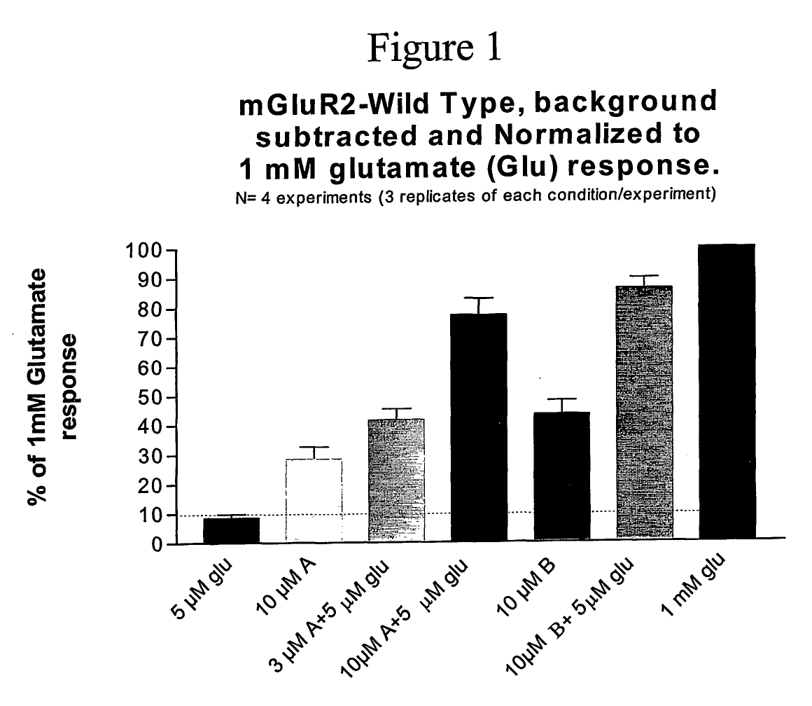

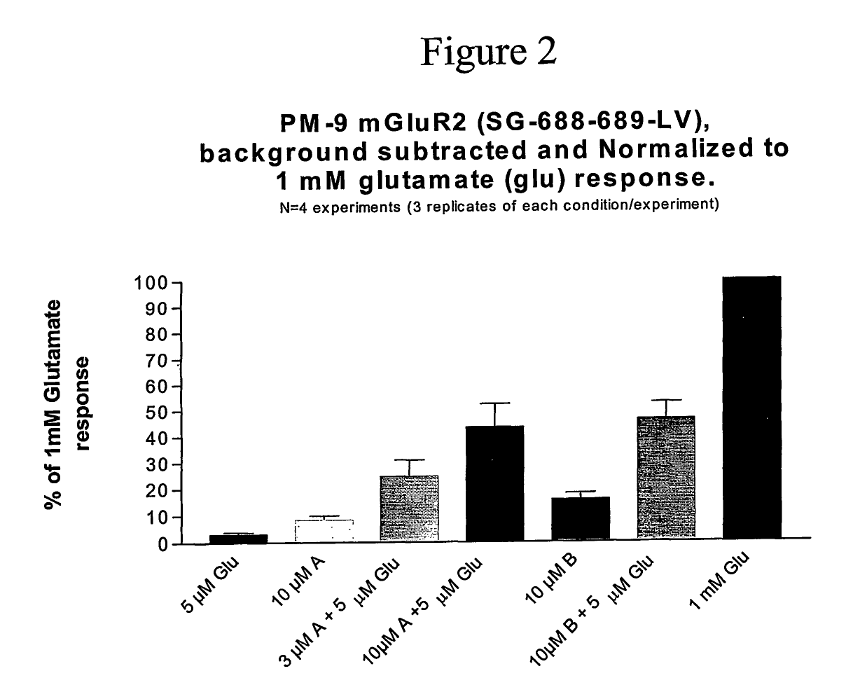

The ability of glutamate to bind to an mGluRx receptor is essential in the development of a multitude of indications. In identifying and developing substances that act as allosteric modulators of a particular mGluRx receptor, such as mGluR2, it would be desirable, therefore, to determine those substances that bind to the allosteric binding site described herein. Generally, such an assay includes a method for determining whether a substance non-competitively affects the activity of the designated mGluRx receptor, and more specifically such an assay compares the effect of said substance on glutamate reception in assay systems using wild type mGluRx, and at least one of the mutant mGluRxs of the present invention. Typically a control of wild-type mGluRx with a known amount of glutamate, i.e., 1 mM, serves as a positive control against which other responses are normalized. For instance, a purported up-modulator of mGluR2 that acts at the allosteric binding site discovered herein is expected to raise the response with a small glutamate quantity (e.g., 3 or 10 μM) in wild-type mGluR2, but is expected to have a diminished up-modulation effect in a treatment having a mutant mGluR2 where the mutant has at least one of any of the following amino acid substitutions: S688L; G689V; and N735D. This difference in modulation can be observed by comparing the effect of modulator Compounds A and B in FIG. 1 with the lower effect of such modulators in FIGS. 2-6.

The instant invention provides such a screening system useful for discovering agents which allosterically modulate glutamate binding to the mGluR2 (or, alternately, the mGluR3) receptor, said screening system comprising the steps of:

a) preparing a human mutant mGluR2 mutant receptor, as with any of the sequences SEQ.ID.NOS.:9-16, and obtaining or preparing a wild-type human mGluR2 receptor;

b) transforming cells to express, separately, the mutant and wild-type mGluR2 receptors prepared in “a)”;

c) introducing glutamate at desired levels in each treatment and control;

d) exposing said human mGluR2 mutant and wild-type receptors (expressed per the transformations in “b)” above) to a potential allosteric modulator of the mGluR2 receptor complex (where said potential allosteric modulator may bind to the site(s) affected by the mutation in the mutant mGluR2); and,

e) quantifying the relative degree of responses among the treatments such as compared to a positive control provided a higher level of glutamate.

This allows one to rapidly screen for allosteric modulators of the glutamate/HmGluR2 receptor complex. This screening approach also is applied to mGluR3. Utilization of the screening system described above provides a sensitive and rapid means to determine compounds which modulate mGluR2 but not mGluR3 to the same extent, or vice versa. For example, a preferred modulator identified with the present invention modulates mGluR2 at a pharmacologically effective dose, but its impact on modulation of mGluR3 is small, for instance, an up-modulation of less than 20 percent, whereas the up-modulation for mGluR2 by said modulator is over 100 percent change under the same test conditions. This increase in specificity afforded by the present invention is seen to result in decrease toxicity by increasing targeted effectiveness at lower doses and eliminating undesired stimulation of non-targeted mGluRx receptors.

Another use of such mutant forms of mGluR2 and mGluR3 is to use as “negative controls” in studies that evaluate whether or not a modulator binds to the corresponding wild-type mGluR2 or mGluR3. In such studies, strong binding to mGluR2 wild-type receptors and substantially less or no binding to mGluR2 mutants at the 688, 689, and/or 735 sites indicates that said modulator has good specificity to an allosteric modulator site on wild-type mGluR2 (where the mutations disrupt the association that provides for the modulator's effect). It further suggests that there would not be cross-over effect to modulate mGluR3 at a corresponding site on mGluR3.

The following table provides a summary of desired levels of changes in the targeted mGluRx compared to the mGluRx that is most affected by the modulator but which is not the targeted subtype:

| | | |
|---|---|---|
| Levels of Modulator | | |
| Specificity (arbitrary | Targeted | Non-targeted mGluRx most |
| scale) | mGluRx | affected by modulator |

For a “positive” modulator - approx.
percent change compared to no modulator

| | | |
|---|---|---|
| 1 | +25% | +15% |
| 2 | +25% | +10% |
| 3 | +50% | +15% |
| 4 | +50% | +10% |
| 5 | +100% | +10% |
| 6 | +200% | +15% |
| 7 | +500% | +25% |
| 8 | >500% | <50% |

For a “negative” modulator - approx.
percent change compared to no modulator

| | | |
|---|---|---|
| 1 | −25% | −15% |
| 2 | −25% | −10% |
| 3 | −50% | −15% |
| 4 | −50% | −10% |
| 5 | −100% | −10% |
| 6 | −200% | −15% |
| 7 | −500% | −25% |
| 8 | <500% | >50% |

For example, considering a negative modulator targeted for mGluR2, where mGluR3 is the non-targeted mGluRx with the highest response of all non-targeted, identified mGluRxs, a level 4 response (“good”) is when the modulator provides about a 50% decrease in glutamate response for mGluR2, and only about a 10% decrease in glutamate response for mGluR3. A better profile for a modulator, in that it is more specific, is a level 5 response, where the modulator provides about a 100% decrease in glutamate response for mGluR2, and about the same 10% decrease in glutamate response for mGluR3.

Screening systems, such as based on the above method, may also be adapted to automated procedures such as a PANDEX.RTM. (Baxter-Dade Diagnostics) system allowing for efficient high-volume screening of potential allosteric modulator therapeutic agents.

It is also noted that an oocyte transient expression system can be constructed according to the procedure described in S. Lubbert, et al, Proceedings of the National Academy of Sciences (USA), 84:4332 (1987).

In an especially preferred embodiment of this invention an assay measuring the production of phosphoinositides was performed. The production of phosphoinositides is known to positively correlated with the addition of glutamate to cells containing certain types of metabotropic receptors that are coupled to Gq or to Gs or Gi linked metabotropic GPCRs that are co-expressed with various promisceous g-proteins that now couple these Gs or Gi linked receptors to Gq-linked second messenger systems.

In an especially preferred embodiment of this invention an assay measuring the inhibition of forskolin-stimulated cAMP synthesis was performed. The inhibition of cAMP synthesis is known to positively correlated with the addition of glutamate to cells containing certain types of metabotropic receptors.

In another embodiment this invention provides a method for identifying, in a test sample, DNA homologous to a probe of the present invention, wherein the test nucleic acid is contacted with the probe under hybridizing conditions and identified as being homologous to the probe. Hybridization techniques are well known in the art. See, e.g., J. Sambrook, et al., supra, at Chapter 11.

The nucleic acid compounds of the present invention may also be used to hybridize to genomic DNA which has been digested with one or more restriction enzymes and run on an electrophoretic gel. The hybridization of radiolabeled probes onto such restricted DNA, usually fixed to a membrane after electrophoresis, is well known in the art. See, e.g., J. Sambrook, supra. Such procedures may be employed in searching for persons with mutations in these receptors by the well-known techniques of restriction fragment length polymorphisms (RFLP), the procedures of which are described in U.S. Pat. No. 4,666,828, issued May 19, 1987, the entire contents of which is incorporated herein by reference.

The determination of the DNA base sequence of the human genome is considered to have a major impact on biomedical science in the next century. It is believed that such determinations will enhance a range of applications from genetic mapping of disease-associated genes to diagnostic tests for disease susceptibility and drug response. Briefly, the determination of base composition at specific, variable DNA sites known as single nucleotide polymorphisms (SNPs) is especially important. SNPs have a number of uses in mapping, disease gene identification, and diagnostic assays. All of these applications involve the determination of bases composition at the SNP site.

Conventional techniques to determine base composition at a single site include minisequencing (See, e.g., “Minisequencing: A Specific Tool For DNA Analysis And Diagnostics On Oligonucleotide Arrays,” by Tomi Pastinen et al., Genome Research 7, 606 (1997)), and oligo-ligation (See, e.g., “Single-Well Genotyping Of Diallelic Sequence Variations By A Two-Color ELISA-Based Oligonucleotide Ligation Assay,” by Vincent O. Tobe et al., Nuclear Acids Res. 24, 3728 (1996)). In minisequencing, a primer is designed to interrogate a specific site on a sample template, and polymerase is used to extend the primer with a labeled dideoxynucleotide. In oligo-ligation, a similar primer is designed, and ligase is used to covalently attach a downstream oligo that is variable at the site of interest. In each case, the preference of an enzyme for correctly base-paired substrates is used to discriminate the base identity that is revealed by the covalent attachment of a label to the primer. In most applications these assays are configured with the primer immobilized on a solid substrate, including microplates, magnetic beads and recently, oligonucleotides microarrayed on microscope slides. Detection strategies include direct labeling with fluorescence detection or indirect labeling using biotin and a labeled streptavidin with fluorescent, chemiluminescent, or absorbance detection.

Oligonucleotide microarrays or “DNA chips” have also generated much attention for their potential for massively parallel analysis. The prospect of sequencing tens of thousands of bases of a small sample in just a few minutes is exciting. At present, this technology has limited availability in that arrays to sequence only a handful of genes are currently available, with substantial hardware and consumable costs. In addition, the general approach of sequencing by hybridization is not particularly robust, with the requirement of significant sequence-dependent optimization of hybridization conditions. Nonetheless, the parallelism of an “array” technology is very powerful and multiplexed sequence determination is an important element of the new flow cytometry method.

Other methods for SNP analysis include those in U.S. Pat. No. 6,287,766.

Briefly, a DNA sequence variation can be analyzed using the techniques outlined in the above referenced patent. In general, differences exist in similar DNA regions isolated from two individuals of a species. Such differences in DNA sequence are known as genetic sequence variation. Genetic sequence variation may result in phenotypic differences in the two individuals or may have no phenotypic effect whatsoever. Similarly, the genetic sequence variation may have a profound effect on the host of the different genetic sequence or it may have no effect whatsoever.

Comparisons between two DNA samples can lead to useful genetic information. For example, with the various genome projects, reference or control sequences are available to use for comparison purposes. New DNA samples isolated from similar or dissimilar organisms can be compared to the known sequences using the techniques of the invention. Similarly, two different unknown samples can be compared.

In general, a first DNA sample is attached to a solid support such as flow cytometry beads. The first DNA sample may be a known sample or an unknown DNA sample. Next, a test sample of DNA is isolated. The test sample may be a PCR product. The test sample is then incubated with the DNA attached to the solid support under conditions suitable to permit DanA hybridization between the two DNA samples. DNA sequence differences are detected as DNA mismatches or other “mutations” in the hybrid DNA.

SNP Analysis

Single Nucleotide Polymorphisms (SNP) can be detected using the techniques disclosed in the U.S. Pat. No. 6,287,766 patent. Importantly, the SNPs can be known or unknown.

1. Known SNPs

Native or wild type DNA is attached to a solid support such as flow cytometry beads. Next, the test DNA sample is isolated. The DNA may be amplified by PCR using oligos that flank the SNP. The DNA sample is incubated with the native or wild type DNA attached to the solid support. Finally, the mixture is incubated with a labeled DNA mutation binding protein. A detected mismatch is indicative of a SNP. The test sample may be isolated from a patient.

In an alternative embodiment, two solid supports are utilized. Native or wild type DNA is coupled to one support. DNA containing the known mutation is coupled to the other support. A DNA sample is incubated with both solid supports under conditions that allows DNA hybridization. Finally, the mixture is incubated with a labeled DNA mutation binding protein. If the sample DNA has a SNP, the native DNA-support will show a mismatch and the mutant DNA-support will show no signal due to the match. If the sample DNA does not have the mutation, the native support will show a match and the mutant support will show a mismatch.

The most efficient solid support for known SNP analysis is flow cytometry. A library of DNA molecules of interest can be coupled to the beads. Such DNA can be produced by PCR. For example, if one is attempting to identify a SNP in a particular gene such as a BRCA breast cancer gene, one would PCR a blood sample from a candidate to amplify the DNA using oligos that flank the region of interest. The PCR sample is then annealed to the beads containing the BRCA native DNA in sections of 100-200 bases with overlap to ensure SNP detection in the entire gene. A DNA mutation binding protein such as thermophilic MutS protein which recognizes all DNA mismatches is labeled with a detectable label such as biotin and is then incubated with the beads. Streptavidin Phycoerythrin can be used as a reporter. If the DNA mutation binding protein (MutS) protein detects a DNA mutation, it binds to the DNA which is bound to the bead forming a DNA mutation binding protein-DNA-bead complex. The biotin attached to the DNA mutation-binding protein (MutS) is detected by the reporter using flow cytometry.

The ability to assay 100 beads (each with a different form of DNA attached) per sample using flow cytometry in a matter of seconds makes the genomic approach to SNP detection feasible. This approach makes it possible to narrow down the SNP to approximately 100 bases which can then be sequenced to identify the exact nature of the SNP or change in the sequence. The DNA sequencing could be done from the initial PCR reaction used to anneal to the beads thus simplifying the SNP detection greatly.

2. Unknown SNPs

For genome wide search for SNPs, flow cytometry is the preferred form of detection. This method proposes attaching wild type DNA (in 200-500 base fragments where each fragment overlaps with the next by about 30 bases to insure that all DNA is read) to a library of beads. Preferably, smaller fragments such 200 bases are used since it is estimated that a SNP occurs every 1000 base pairs and it is desirable to narrow down the SNP to limit the amount of sequencing needed. As above, the patient sample is amplified and annealed to the beads containing the wild type DNA sequence.

One hundred (100) beads (which can read in mutliplex fashion—at the same time due to the ability to detect each bead by its fluorescent signature) can be read in one test well. Therefore in 96 well format, 9600 sections of 200 bases can be read in less than an hour which correlates with 1,920,000 bases checked for SNPs on each 96 well plate. Mismatches can be detected in minutes thereby reflecting a SNP in the respective 200 base fragment on that bead. If that fragment corresponds to a DNA sequence of interest, further sequencing (from the original PCR sample used to anneal to the beads) will identify the exact SNP sequence.

The identity of the herein disclosed nucleic acid molecules will also find use in the identification of individuals harboring identical or substantially similar sequences in their genomes. This, finding, in turn, will aid the practicing physician in identifying those individuals at risk of not responding to medicaments aimed at normalizing endogenous glutamate levels especially those seeking treatment of mGluR2 or mGluR3 related disorders characterized with low or non-existent levels of endogenous glutamate.

Thus, a diagnostic method for screening an individual at risk for non-compliance with an mGluR2/3 related therapeutic regimen comprises:

(a) providing a biological sample of human cell/tissue; and

(b) determining, in the sample, levels of expression of a gene product expressed from a nucleotide sequence which hybridizes under stringent conditions with a nucleotide sequence corresponding to one or more of SEQ.ID.NOS.:9-16, or SEQ.ID.NOS.:41-48, or a complement thereof, wherein said step comprises identifying a complex formed between said gene product and an antibody having affinity for said gene product.

An alternative method proposes assaying a biological sample from a subject undergoing a therapeutic regiment to treat an mGluR2/3 related disorder characterized by abnormal binding of glutamate to its respective receptor, for expression of mRNA of a gene comprising a sequence of nucleotides as set forth in SEQ.ID.NOS.:9-16, 25-32 or 41-48 or substantially identical thereto or encoding the polypeptide of SEQ.ID.NOS.:1-8, 17-24 or 33-40, wherein expression thereof is indicative of the presence of a mutant form of mGluR2 or 3 as disclosed herein. This finding, in turn, will aid in the identifications of individuals at risk of not responding to a therapeutic regiment aimed at correcting/normalizing levels of endogenous glutamate. In further thereto, it is believed that individuals having nucleotides sequences identical to or substantially similar to SEQ.ID.NOS.:9-16, 25-32 or 41-48 will have lower binding of a selective allosteric potentiator which would result in less potentiation by the endogenous glutamate which would eventually result in less inhibition of the presynaptic mGluR2 receptor resulting in increased synaptic activity of the presynaptic terminal.

The proteins of this invention as well as fragments of these proteins may be used as antigens for the synthesis of antibodies. The term “antibody” as used herein describes antibodies, fragments of antibodies (such as, but not limited, to Fab, Fab′, Fab.sub.2′, and Fv fragments), and chimeric, humanized, veneered, resurfaced, or CDR-grafted antibodies capable of binding antigens of a similar nature as the parent antibody molecule from which they are derived. The instant invention also encompasses single chain polypeptide binding molecules.

The term “antibody” as used herein is not limited by the manner in which the antibodies are produced, whether such production is in situ or not. The term “antibody” as used in this specification encompasses those antibodies produced by recombinant DNA technology means including, but not limited, to expression in bacteria, yeast, insect cell lines, or mammalian cell lines.

The production of antibodies, both monoclonal and polyclonal, in animals, especially mice, is well known in the art. See. e.g., C. Milstein, Handbook of Experimental Immunology, (Blackwell Scientific Pub., 1986); J. Goding, Monoclonal Antibodies: Principles and Practice, (Academic Press, 1983). For the production of monoclonal antibodies the basic process begins with injecting a mouse, or other suitable animal, with an immunogen. The mouse is subsequently sacrificed and cells taken from its spleen are fused with myeloma cells, resulting in a hybridoma that reproduces in vitro. The population of hybridomas is screened to isolate individual clones, each of which secretes a single antibody species, specific for the immunogen. The individual antibody species obtained in this way is each the product of a single B cell from the immune animal generated in response to a specific antigenic site, or epitope, recognized on the immunogenic substance.

Chimeric antibodies are described in U.S. Pat. No. 4,816,567, which issued Mar. 28, 1989 to S. Cabilly, et al. This reference discloses methods and vectors for the preparation of chimeric antibodies. The entire contents of U.S. Pat. No. 4,816,567 are incorporated herein by reference. An alternative approach to production of genetically engineered antibodies is provided in U.S. Pat. No. 4,816,397, which also issued Mar. 28, 1989 to M. Boss, et al., the entire contents of which are incorporated herein by reference. The Boss patent teaches the simultaneous co-expression of the heavy and light chains of the antibody in the same host cell.

The approach of U.S. Pat. No. 4,816,397 has been further refined as taught in European Patent Publication No. 0 239 400, which published Sep. 30, 1987. The teachings of this European patent publication (Winter) are a preferred format for the genetic engineering of the reactive monoclonal antibodies of this invention. The Winter technology involves the replacement of complementarity determining regions (CDRS) of a human antibody with the CDRs of a murine monoclonal antibody thereby converting the specificity of the human antibody to the specificity of the murine antibody which was the source of the CDR regions. This “CDR grafting” technology affords a molecule containing minimal murine sequence and thus is less immunogenic.

Single chain antibody technology is yet another variety of genetically engineered antibody which is now well known in the art. See. e.g. R. E. Bird, et al., Science 242:423-426 (1988); PCT Publication No. WO 88/01649, which was published Mar. 10, 1988. The single chain antibody technology involves joining the binding regions of heavy and light chains with a polypeptide sequence to generate a single polypeptide having the binding specificity of the antibody from which it was derived.

The aforementioned genetic engineering approaches provide the skilled artisan with numerous means to generate molecules which retain the binding characteristics of the parental antibody while affording a less immunogenic format.

These antibodies are used in diagnostics, therapeutics or in diagnostic/therapeutic combinations. By “diagnostics” as used herein is meant testing that is related to either the in vitro or in vivo diagnosis of disease states or biological status in mammals, preferably in humans. By “therapeutics” and “therapeutic/diagnostic combinations” as used herein is respectively meant the treatment or the diagnosis and treatment of disease states or biological status by the in vivo administration to mammals, preferably humans, of the antibodies of the present invention. The antibodies of the present invention are used in the screening methods and in rational drug development, as well as in other applications where the immunological binding of an antibody to one of the novel polypeptides, peptides or fused proteins is desired.

Numerous other assay systems are also readily adaptable to detect agents which bind the novel polypeptides, peptides or fused proteins of the present invention. Examples of these aforementioned assay systems are discussed in Methods in Enzymology, (J. Langone. and H. Vunakis, eds. 1981), Vol. 73, Part B, the contents of which are incorporated herein by reference. Skilled artisans are directed to Section II of Methods in Enzymology, Vol.73, Part B, supra, which discusses labeling of antibodies and antigens, and Section IV, which discusses immunoassay methods. Also, regarding the development of other antibodies, which are specific for the hypervariable regions of the mGluRx receptor antibodies, wherein some such anti-idiotypic antibodies would resemble the original epitope, and, therefore, would be useful in evaluating the effectiveness of compounds which are potential antagonists, agonists, or partial agonists of the mGluRx receptor itself (as opposed to the allosteric site), see. e.g., Cleveland, et al., Nature (London), 305:56 (1983); Wasserman, et al., Proceedings of the National Academy of Sciences (USA), 79:4810 (1982).

Several methods for preparing and using the compounds, and applying the methods, of this invention are illustrated in the following Examples. Starting materials are made according to procedures known in the art or as illustrated herein.

### EXAMPLE 1

Site Directed Mutagenesis Using the QuickChange Protocol of Stratagene™:

The following summarizes the procedure that is employed to generate mutant forms of mGluR2 and mGluR3 at the specific amino acid locations indicated above. This is based on the protocol developed by Stratagene™.

1. Resuspend all primers in sterile water with a final concentration of 1 ug/ul. Make working stocks of each primer by diluting to 125 ng/ul.

2. Prepare test reaction and positive control reaction according to table below, using components provided within the kit:

| Reactants: | Test reaction | | Control | |
|---|---|---|---|---|
| 10× Buffer | 5 | ul | 5 | ul |
| Template 20 ng/ul | 1 | ul hmGluR2 | 1 | ul pWhitescript |
| Primer 1 125 ng/ul | 1 | ul hmG2 Primer 1 | 1.25 | ul primer 1 |
| Primer 2 125 ng/ul | 1 | ul hmG2 Primer 2 | 1.25 | ul primer 2 |
| dNTP | 1 | ul | 1 | ul |
| Water | 40 | ul | 38.5 | ul |
| PfuTurbo | 1 | ul | 1 | ul |

1. Place samples in PCR thermo cycler and set the following PCR Cycling Conditions:
1 Cycle: 95° C.-30 seconds
18 Cycles: 95° C.-30 seconds
   55° C.-60 seconds
   68° C.-12 minutes
Hold: 4° C.

2. Add 1 ul DpnI enzyme to each reaction and incubate for 1 hour at 37° C.

3. Prepare LB/AMP plates.

4. Add 10 ul 0.1M IPTG and 50 ul 40 mg/ml XGal per plate and spread evenly.

5. Let plates sit in 37° C.-incubator 1 hour before using.

6. Thaw competent cells on ice.

7. Transfer 50 uL of competent cells to pre-chilled falcon tubes, 1 tube per reaction.

8. Add 1 uL of DpnI treated reactions to the separate tubes and incubate on ice for 30 minutes.

9. Heat transformation in 42° C.-water bath for 45 seconds, then cool on ice for 2 minutes.

10. Add 500 uL NZY+ broth (42° C.). Incubate at 37° C. for 1 hour at 225 rpm.

11. Plate 250 ul of transformations per plate. Incubate overnight at 37° C.

12. Remove plates from 37° C. and store at 4° C.

13. Pick colonies from each clone and inoculated them into 4 ml LB/AMP. Incubate overnight at 37° C., 300 rpm.

Then DNA is isolated from overnight cultures using a mini-prep kit (Qiagen™ or equivalent) following the kit instructions. Samples of DNA are sequenced by standard sequencing protocols to determine if a desired, correct mutation has been incorporated into the sequence and no further mutations have been mis-incorporated.

### EXAMPLE 2

Lipid Transfection to Introduce Mutant DNA into Cells Using MAGEC™ Multi-Well Plate-Based Protocol:

For mGluR2 and mGluR3 mutants, the mGluR2/Gα16 nucleic acids are mixed with a lipid-based transfection reagent and are deposited into a well of a multi-well plate coated with fibronectin. The resulting DNA/lipid complexes are subsequently dried in vacuo then seeded with HEK293 cells. This is based on a method designated as MAGECTM, and disclosed in U.S. Provisional Application No. 60/372,476, filed Apr. 15, 2002, and titled, “Matrix Analyses of Gene Expression in Cells.”

The following summarizes the steps of the method:

1. In a 15 ml tube, add 0.35 μg DNA of mGluR2 and Gα16 DNAs to 45 μl of DNA-condensation buffer (Buffer EC from Effectene Kit, Qiagen, Inc.) in which sucrose has been dissolved to a concentration of 0.3 M.

2. Add 4.5 μl of Enhancer solution, mix the contents by pipetting up vortex gently then incubate the mixture at room temperature for 5 minutes.

3. Add 11 μl Effectene transfection reagent; mix the solution with gentle vortexing.

4. Incubate at room temperature for 10 minutes.

5. Add 90 μl 0.25% glycogen, remix the solution and pipette 150 μl into a well of multi-well fibronectin coated plate.

6. Spread solution to cover the whole bottom of the well and let sit overnight at 4° C.

7. Dry plate in a SpeedVac or vacuum chamber. Dried plates can be used immediately or stored at 4° C. for an extended period.

8. One day prior to transfection, cells are passaged to maintain growth in log-phase.

9. On the day of transfection, cells are trypsinized and 600,000 cells/well are subsequently seeded into the previously coated and dried multi-well plate(s) in DMEM media containing glutamine and placed at 37° C. with 6% CO₂.

10. Approximately 26-30 hours after plating, the media is removed from the cells and 1 ml of DMEM media minus glutamine is pipetted over the cells and the plates are placed back at 37° C. with 6% CO₂.

11. Approximately forty hours after plating, the cells are analyzed in the phosphoinositide hydrolysis assay.

### EXAMPLE 3

### Measurement of Phosphoinositide Hydrolysis

Phosphoinositide hydrolysis was determined by measuring the accumulation of tritiated inositol monophosphate ([³H]-IP₁) in the presence of LiCl. The method is taken from Berridge: Biochem J (1983) 212: 849-58. This determination provides for comparative data on the level of potentiation and loss of potentiation when testing a purported or known allosteric modulator in the presence of glutamate and either a wild-type mGluR2 or mGluR3 and at least one mutant of the respective mGluR2 or mGluR3.

1. HEK293 cells that were previously transiently transfected with hmGluR2 and Gα16 were labeled overnight with 1 μCi/well myo-[2-³H]-inositol in a glutamine-free DMEM medium.

2. The following day, the medium was removed and the cells were washed two times with HBS buffer containing 125 mM NaCl. 5 mM KCl, 0.62 mM MgSO₄, 1.8 mM CaCl₂, 6 mM glucose and 20 mM HEPES pH 7.4 for 45 min at 37° C.

3. Following the wash, cells were then incubated with HBS-buffer containing 10 mM LiCl.

4. After 20 min incubation in the LiCl containing buffer, varying concentrations of glutamate alone (5 uM (EC₁₀) or 1 mM (EC₁₀₀)), or glutamate (5 uM (EC₁₀) in combination with varying concentrations of a mGluR2 potentiator were added to the wells and incubated for an additional hour at 37° C.

5. The reactions were terminated by aspiration of the media from the wells and the accumulated [³H]-inositol monophosphates were extracted by adding 1 ml of cold chloroform-methanol-HCl (4N) (200:100:2).

6. The mixtures were then transferred to glass tubes containing 300 μl of chloroform and 400 μl of H₂O and vortexing briefly.

7. The aqueous phase was separated from the organic phase, by centrifugation at 4000 rpm for 3 min or the samples can be allowed to settle for 15 min.

8. A 0.5 ml aliquot of the aqueous phase from each sample was added to an anion exchange column containing Dowex-1-X8 (200-300 mesh in the formate form).

9. After the application of the sample to the column, the columns were washed with 10 ml of a solution consisting of 60 mM ammonium formate and 5 mM borax.

10. [³H]-inositol monophosphates were eluted from the columns by the addition of 4 ml of a solution containing 800 mM ammonium formate and 0.1 M formic acid.

11. The column eluates were transferred to a 20 ml glass scintillation vial and 16 ml of scintillation cocktail (Ecolume, ICN) was added.

12. The sample was quantified in a scintillation counter after a two-hour waiting period.

### EXAMPLE 4

The responses to novel nucleic acid sequences of the present invention were evaluated through comparisons with two known mGluR2 modulators. One of these modulators is designated herein as Compund A, has the chemical formula N-(3-(2-methoxyphenoxy)-phenyl-N-(2,2,2-trifluoroethylsulfonyl)pyrid-3-ylmethylamine, and is described in the PCT Patent Application No.: WO 01/56990. The second modulator is designated as Compound B.

The interactions between the novel mutant forms of mGluR2 and these modulators indicate the value of the novel nucleic acid sequences and polypeptides of the present invention for purposes including, but not limited to:

1. screening of potential mGluR2 modulators that are not active, or less active, for mGluR3;

2. screening of potential mGluR3 modulators that are not active, or less active, for mGluR2;

3. rational drug design using SAR to design specific modulators for mGluR2 that are not active, or less active, for mGluR3; and

4. rational drug design using SAR to design specific modulators for mGluR3 that are not active, or less active, for mGluR2.

FIG. 1 shows the percent response of several treatments compared to a 1 mM glutamate positive control with wild type mGluR2. This control is normalized and set to “100 percent on the y-axis. This figure shows the effect of the two modulators, Compound A and Compound B, in up-regulating the response of mGluR2 when provided a relatively low level, 5 uM of glutamate (“glu”), compared to the 1 mM level of the positive control (far right bar).

FIG. 2 shows the effect of the SG-688-689-LV mutant of mGluR2, (SEQ.ID.NO.:5), when the same modulators are applied. A substantial decrease of glutamate response is evident in comparison to the wild-type mGluR2 results in FIG. 1. This indicates that the modulators which were effective at up-regulating wild-type mGluR2 in the presence of 5 uM quantities of glutamate (relative to the 1 mM control) are both less effective at such up-modulation when acting on the SG-688-689-LV mutant of mGluR2.

FIG. 3 shows the effect of the N735D mutant of mGluR2, (SEQ.ID.NO.:1), when the same modulators are applied. A substantial decrease of glutamate response is evident in comparison to the wild-type mGluR2 results in FIG. 1. This indicates that the modulators which were effective at up-regulating wild-type mGluR2 in the presence of 5 uM quantities of glutamate (relative to the 1 mM control) are both less effective at such up-modulation when acting on the N735D mutant of mGluR2. It also is noted that the loss of effectiveness of these modulators is somewhat greater for this mutant than for the mutant of FIG. 2.

FIG. 4 shows the effect of four combined point mutations—the SG-688-689-LV and AN(733,735)TD mutant of mGluR2, (SEQ.ID.NO.:8) when the same modulators are applied. A more substantial decrease of glutamate response is evident in comparison to the wild-type mGluR2 results in FIG. 1. This indicates that the modulators which were effective at up-regulating wild-type mGluR2 in the presence of 5 uM quantities of glutamate (relative to the 1 mM control) are both less effective at such up-modulation when acting on the SG-688-689-LV and AN(733,735)TD mutant of mGluR2. It also is noted that the loss of effectiveness of these modulators is greater for this mutant than for the mutants of FIGS. 2 and 3. This suggests that this combination of mutations is more effective at disrupting an allosteric binding site for these modulators.

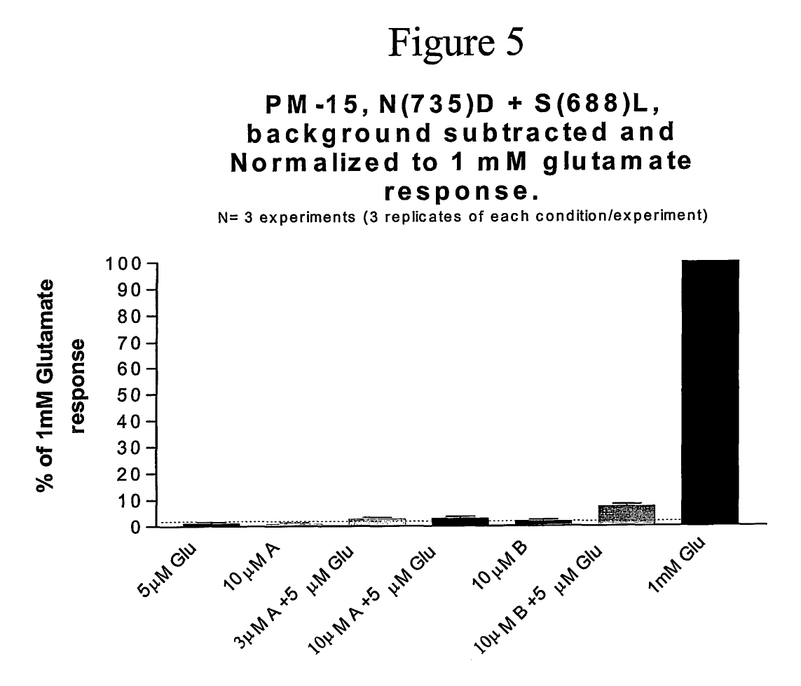

FIG. 5 shows the effect of two combined point mutations—the S-688-L and N735D mutant of mGluR2, (SEQ.ID.NO.:6), when the same modulators are applied. A more substantial decrease of glutamate response is evident in comparison to the wild-type mGluR2 results in FIG. 1. This indicates that the modulators which were effective at up-regulating wild-type mGluR2 in the presence of 5 uM quantities of glutamate (relative to the 1 mM control) are both less effective at such up-modulation when acting on the S-688-L and N735D mutant of mGluR2. It also is noted that the loss of effectiveness of these modulators is greater for this mutant than for the mutants of FIGS. 2 and 3, and comparable to the results of FIG. 4. This suggests that this combination of mutations is more effective than the single point mutations, and as effective as the four-mutation combination depicted in FIG. 4.

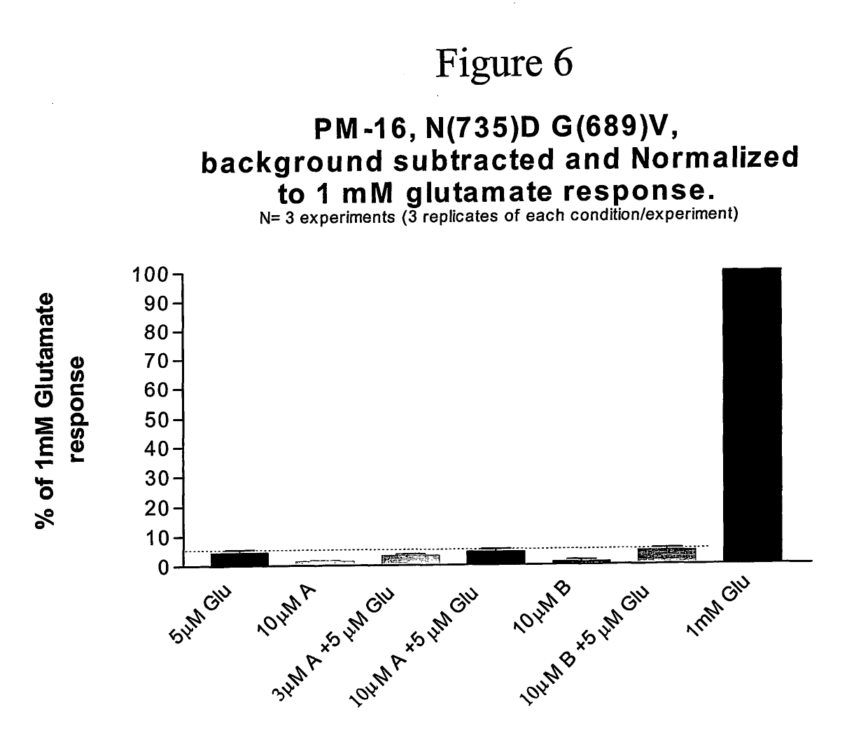

FIG. 6 shows the effect of two combined point mutations—the G-689-V and N735D mutant of mGluR2, (SEQ.ID.NO.:2), when the same modulators are applied. A more substantial decrease of glutamate response is evident in comparison to the wild-type mGluR2 results in FIG. 1. This indicates that the modulators which were effective at up-regulating wild-type mGluR2 in the presence of 5 uM quantities of glutamate (relative to the 1 mM control) are both less effective at such up-modulation when acting on the G-689-V and N735D mutant of mGluR2. It also is noted that the loss of effectiveness of these modulators is greater for this mutant than for the mutants of FIGS. 2 and 3, and comparable to the results in FIG. 4. This suggests that this combination of mutations is more effective than the single point mutations, and as effective as the four-mutation combination depicted in FIG. 4.

Without being bound to a specific theory, the above sets of results suggest that the amino acids at positions 688, 689, and 735 of mGluR2 are important in establishing an allosteric binding site for modulators that include the Compound A and Compound B modulators. The data indicate that substitution of amino acids in these positions dramatically affect the ability of such modulators to act on such mutated mGluR2 receptor molecules. Further, FIGS. 5 and 6 suggest that two mutations one at 735 and one at either 688 or 689, is essentially as effective as a mutant having four mutations. This adds to the knowledge of what structure is critical for the binding and activity of Compounds A and B, and similar modulators. This opens opportunities to exploit the discovery of these positions as critical to effectiveness of modulators, and to develop specific modulators.

Accordingly, the teachings of the present invention find application in a number of areas, some of which are described in this and the following paragraphs. For example, without being limiting of the scope of the present invention, the mutant forms of mGluR2 and mGluR3 disclosed herein are incorporated into chimeric nucleic acid sequences of human, rat, and mouse mGluR2 and/or mGluR3. The resultant chimeric mutant nucleic acids sequences, and the resultant polypeptides, are utilized in screening for modulators, testing of identified modulators for specificity, toxicity testing, and rational drug design and development. With regard to screening of substances for modulator properties, these mutant chimeric molecules, and the resultant polypeptides, are used in high-throughput functional drug screening assays in order to identify subtype-specific modulators, particularly positive modulators, of mGluR2, and of mGluR3.

Further regarding identification of the chimeric mutant nucleic acid sequences and polypeptides encoded by said sequences, novel polypeptides, peptides, and fusion proteins are prepared wherein such polypeptides, peptides, and fusion proteins identify the polypeptides encoded by said sequences. Also, novel nucleic acid sequences are prepared that encode these novel polypeptides, peptides, and fusion proteins. Also, expression vectors expressing these novel nucleic acid sequences are prepared.

In certain embodiments, these novel polypeptides, peptides, and fusion proteins are designed and constructed to specifically identify a unique or rare sequence of a specific mutant chimeric polypeptide of the present invention. Alternately, in other embodiments, novel polyclonal or monoclonal antibodies are prepared and purified to immunologically bind to said chimeric polypeptide. The specificity of such antibodies is selected to meet the purpose of the antibody/peptide binding requirements for a specific assay or other application. In more stringent specificity applications, the antibody is prepared to immunologically bind to the site of interaction of the modulator, e.g., at the site modified by the mutant forms of mGluR2 or mGluR3 of the present invention.

In other embodiments, the corresponding sequences in other species are utilized for assays, comparison and evaluations with in vitro, in vivo cellular, in vivo whole organism (e.g., chimeric), and in vivo site-specific use of mutants of the present invention. In any of such embodiments, the following sequences are of utility: mutant rat mGluR2 peptides, SEQ.ID.NOs.:17-24; mutant rat mGluR2 DNA nucleic acid sequences, SEQ.ID.NOS.:25-32; mutant human mGluR3 peptides, SEQ.ID.NOS.:33-40; and mutant human mGluR3 nucleic acid sequences, SEQ.ID.NOS.:41-48.

In addition, the three sites of mutations in mGlu2 or mGluR3, and optionally nearby relevant amino acids that also are shown to play a role in the allosteric binding of a modulator in the TM4 and TM5 regions associated with these three sites of mutations, are candidates for Single Nucleotide Polymorphism (SNP) analyses. Such comparative pharmacogenetics are conducted either in the general population (to obtain baseline data) and/or in sub-populations of subjects in need of modulators for prevention or treatment of certain neurological conditions, such as schizophrenia and general anxiety disorder.

The above additional application of the present invention are developed through techniques known to those skilled in the art using methods described in the present application and supported by the information incorporating by reference from the patents, patent applications, publications, texts and references discussed or cited herein.

More generally, all patents, patent applications, publications, texts and references discussed or cited herein are incorporated by reference to the same extent as if each individual publication or patent application was specifically and individually set forth in its entirety, and any such incorporation is not limited in any way to the particular aspect or method for which a patent, patent application, publication, text or reference was cited herein. Nothing herein is to be construed as an admission that the invention is not entitled to antedate such disclosures by virtue of prior invention. In addition, all terms not specifically defined are first taken to have the meaning given through usage in this disclosure, and if no such meaning is inferable, their normal meaning. Where a limitation is described but not given a specific term, a term corresponding to such limitation may be taken from any references, patents, applications, and other documents cited herein, or, for an application claiming priority to this application, additionally from an Invention Disclosure Statement, Examiner's Summary of Cited References, or a paper otherwise entered into the file history of this application.

Additional references useful in understanding aspects of the present disclosure and invention includes: D. D. Schoepp, “Glutamate receptors”, Handbook of Receptors and Channels, Chapter 13 (S. J. Peroutka, ed., CRC Press, 1984); ESCRIBANO, A. et al., “(2S,4S)-2-Amino-4(2,2-Diphenylethyl) Pentanedioic Acid Selective Group 2 Metabotropic Glutamate Receptor Antagonist”, Bioorg. Med. Chem. Lett., 1998, 765-770:8; HELTON, DAVID R., et al., “Anxiolytic and Side-Effect Profile of LY354740: A Potent, Highly Selective, Orally Active Agonist for Group II Metabotropic Glutamate Receptors”, JPET, 1998, 651-760: 284, USA;

KINGSTON, A. E., et al., “LY341495 is a Nanomolar Potent and Selective Antagonist of Group II Metabotropic Glutamate Receptors”, Neuropharmacology, 1998, 1-12: 37; MUKHOPADHYAYA, J. K., et al., “Synthesis of N¹-Substituted Analogues of (2R, 4R)-4-Amino-pyrrolidine-2,4-dicarboxylic Acid as Agonists, Partial Agonists, and Antagonists of Group II Metabotropic Glutamate Receptors”, Bioorg. Med. Chem. Lett., 2001, 1919-1924: 11; NAKAZATO, A. et al., “Synthesis, SARS, and Pharmacological Characterization of 2-Amino-3 or 6-fluorobicyclo[3.1.0] hexane-2,6-dicarboxylic Acid Derivatives as Potent, Selective, and Orally Active Group II Metabotropic Glutamate Receptor Agonists”, J. Med. Chem., 2000, 4893-4909: 43, 25; NAPLES, M. A. & HAMPSON D. R., “Pharmacological Profiles of the Metabotropic Glutamate Receptor Ligands [³H]L-AP4 and [³H] CPPG”, Neuropharmacology, 2001, 170-177: 40; ORNSTEIN, P. L. et al., “2-Substituted (2SR)-2-Amino-2-((1SR,2 SR)-carboxycycloprop-1-yl) glycines as Potent and Selective Antagonists of Group II Metabotropic Glutamate Receptors. 1. Effects of Alkyl, Arylalkyl, and Diarylalkyl Substitution”, J. Med. Chem., 1998, 346-357: 41.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims. Thus, for the above variations and in other regards, it should be understood that the examples and embodiments described herein are for illustrative purposes only and that various modifications or changes in light thereof will be suggested to persons skilled in the art and are to be included within the spirit and purview of this application and the scope of the appended claims.

Also, although only a few exemplary embodiments of this invention have been described in detail above, those skilled in the art will readily appreciate that many modifications are possible without undue experimentation based on the methods and citations provided, and based on the exemplary embodiments herein, without materially departing from the novel teachings and advantages of this invention. Accordingly, all such modifications are intended to be included in the scope of this invention as defined in the following claims. In the claims, means-plus-function clauses and step-plus-function clauses are intended to cover the structures described herein as effectuating or performing the recited function and to cover not only structural equivalents, but also to cover equivalent structures as one of ordinary skill in the art would understand equivalence with regard to a any means or any step that will achieve a stated function in an equivalent manner. For instance, a “means to detect a modulator of mGluR2” should be taken to include methods now or later known to those of skill in the art regardless of differences in the exact steps and reagents required to achieve this function.

1. An isolated nucleic acid encoding a mutant subtype 2 metabotropic glutamate receptor which comprises an amino acid sequence selected from the group consisting of SEQ.ID.NOS.:1-7, and SEQ.ID.NO.:8, wherein said.

2. A composition comprising an isolated nucleic acid containing a sequence encoding a mutant subtype 2 metabotropic glutamate receptor as claimed in claim 1, wherein said isolated nucleic acid sequence is selected from the group consisting of:
a) SEQ.ID.NOS.:9-16;
b) a nucleic acid compound complementary to any sequence of (a); and
c) a fragment of (a) or (b) that is at least 144 base pairs in length which will selectively hybridize to human genomic DNA encoding a human metabotropic glutamate receptor, and which will encode for the section of the mutant mGluR2 comprising altered amino acids in at least one of positions 688, 689 and 735.

3. (canceled)

4. (canceled)

5. (canceled)

6. (canceled)

7. (canceled)

8. An expression vector a comprising the nucleic acid of claim 2 in combination with regulatory elements necessary for expression of the nucleic acid in a suitable host cell.

9. The expression vector of claim 8, wherein the host cell is of mammalian origin.

10. (canceled)

11. (canceled)

12. (canceled)

13. (canceled)

14. (canceled)

15. (canceled)

16. An isolated mutant mGluR2 receptor which comprises the amino acid sequence selected from the group consisting of SEQ ID NOS: 1-8, wherein said mutant mGluR2 is characterized as being capable of depotentiating glutamate receptor activity relative to wild-type mGluR2.

17. A method for producing a mutant mGluR2 protein comprising the steps of:
a) expressing a nucleic acid molecule comprising a sequence of nucleotides that encode a recombinant mutant protein comprising an amino acid sequence is selected from the group consisting of SEQ ID NOS: 1-8, in a suitable host cell under conditions favoring expression of said mutant protein; and
b) purifying said recombinant protein by any suitable method.

18. (canceled)

19. A substantially pure mutant metabotronic glutamate receptor protein designated mGluR2 comprising an amino acid substitution at a selected position within an amino acid sequence sufficient to enable said mutant to depotentiate glutamate receptor activity by altering an allosteric site associated with a transmembrane region of said mGluR2 relative to wild-type mGluR2.

20. The mutant mGluR2 of claim 19, wherein said amino acid substitution comprises a substitution of leucine for serine at amino acid position 688 as set forth in SEQ ID NO:4 and said mutant protein depotentiates glutamate receptor activity by altering an allosteric site associated with transmembrane region 4 of said mGluR2.

21. The mutant mGluR2 of claim 19, wherein said amino acid substitution comprises a substitution of valine for glycine at amino acid position 689 as set forth in SEQ ID NO:3 and said mutant protein depotentiates glutamate receptor activity by altering an allosteric site associated with transmembrane region 4 of said mGluR2.

22. The mutant mGluR2 of claim 19, wherein said amino acid substitution comprises a substitution of leucine for serine at amino acid position 688 and aspartic acid for asparagine at amino acid position 735 of SEQ ID NO:6 and said mutant protein depotentiates glutamate receptor activity by altering an allosteric site associated with transmembrane regions 4 and 5 of said mGluR2.

23. The mutant mGluR2 of claim 19, wherein said amino acid substitution comprises a substitution of valine for glycine at amino acid position 689 and aspartic acid for asparagine at amino acid position 735 of SEQ ID NO:2 and said mutant protein depotentiates glutamate receptor activity by altering an allosteric site associated with transmembrane regions 4 and 5 of said mGluR2.

24. The mutant mGluR2 of claim 19, wherein said amino acid substitution comprises a substitution of leucine for serine at amino acid position 688 valine for glycine at amino acid position 689 and aspartic acid for asparagine at amino acid position 735 of SEQ ID NO:3 and said mutant protein depotentiates glutamate receptor activity by altering an allosteric site associated with transmembrane regions 4 and 5 of said mGluR2.

25. The mutant mGluR2 of claim 19, wherein said amino acid substitution comprises a substitution of leucine for serine at amino acid position 688 and valine for glycine at amino acid position 735 of SEQ ID NO:5 and said mutant protein depotentiates glutamate receptor activity by altering an allosteric site associated with transmembrane region 4 of said mGluR2.

26. The mutant form mGluR2 of claim 19, wherein said amino acid substitution comprises a substitution of leucine for serine at amino acid position 688 valine for glycine at amino acid position 689 threonine for alanine at amino acid position 733 of said mGluR2, and aspartic acid for asparagine at amino acid position 735, of SEQ ID NO:8 and said mutant protein depotentiates glutamate receptor activity by altering an allosteric site associated with transmembrane regions 4 and 5 of said mGluR2.

27. The isolated nucleic acid molecule of claim 1 comprising a sequence of nucleotides selected from the group consisting of SEQ.ID.NOS.:9-16.

28. The isolated nucleic acid molecule of claim 27 further comprising a nucleotide sequence encoding a polypeptide fused to an amino or carboxy terminal of said isolated nucleic acid molecule.

29. (canceled)

30. (canceled)

31. (canceled)

32. The mutant mGluR2 of claim 19, wherein said amino acid substitution comprises substitution of aspartic acid for asparagine at amino acid position 744 of SEQ ID NO:33 and said mutant protein depotentiates glutamate receptor activity by altering an allosteric site associated with transmembrane region and 5 of said mGluR3.

33. A substantially pure mutant metabotropic glutamate receptor protein designated mGluR2 comprising an amino acid substitution at a selected position within an amino acid sequence sufficient to enable said mutant to depotentiate glutamate receptor activity by altering an allosteric site associated with a transmembrane region of said mGluR3 relative to wild-type mGluR3.

34. The mutant mGluR3of claim 33 herein said amino acid substitution comprises a substitution of valine for glycine at amino acid position 698 as set forth in SEQ ID NO:39 and said mutant protein depotentiates glutamate receptor activity by altering an allosteric site associated with transmembrane region 4 of said mGluR3.

35. The mutant mGluR3 of claim 33, wherein said amino acid substitution comprises a substitution of leucine for serine at amino acid position 688 and aspartic acid for asparagine at amino acid position 735 of SEQ ID NO: 38 and said mutant protein depotentiates glutamate receptor activity by altering an allosteric site associated with transmembrane regions 4 and 5 of said mGluR3.

36. The mutant mGluR3 of claim 33, wherein said amino acid substitution comprises substitution of valine for glysine at amino acid position 689 and aspartic acid for asparagine at amino acid position 735 of SEQ ID NO: 34 and said mutant protein depotentiates glutamate receptor activity by altering an allosteric site associated with transmembrane regions 4 and 5 of said mGluR3.

37. The mutant mGluR3 of claim 33, wherein said amino acid substitution comprises a substitution of leucine for serine at amino acid position 688, a substitution of valine for glysine at amino acid position 689, and a substitution of aspartic acid for asparagine at amino acid position 735 of SEQ ID NO: 35 and said mutant protein depotentiates glutamate receptor activity by altering an allosteric site associated with transmembrane regions 4 and 5 of said mGluR3.

38. The mutant mGluR3 of claim 33, wherein said amino acid substitution comprises a substitution of leucine for serine at amino acid position 688, and a substitution of valine for glysine at amino acid position 689 as set forth in SEQ ID NO: 37 and said mutant protein depotentiates glutamate receptor activity by altering an allosteric site associated with transmembrane region 4 of said mGluR3.

39. The mutant receptor mGluR3 of claim 33, wherein said amino acid substitution comprises a substitution of leucine for serine at amino acid position 688, a substitution of valine for glysine at amino acid position 689, a substitution of threonine for alanine at amino acid position 733, and a substitution of aspartic acid for asparagine at amino acid position 735 as set forth in SEQ ID NO: 40 and said mutant protein depotentiates glutamate receptor activity by altering an allosteric site associated with transmembrane regions 4 and 5 of said mGluR3.

40. (canceled)

41. (canceled)

42. (canceled)

43. The mutant mGluR2 of claim 19, wherein said amino acid substitution comprises substitution of aspartic acid for asparagine at amino acid position 735 as set forth in SEQ ID NO:1 and said mutant protein depotentiates glutamate receptor activity by altering an allosteric site associated with transmembrane region 5 of said mGluR2.

44. The mutant mGluR3of claim 33 herein said amino acid substitution comprises substitution of leucine for serine at amino acid position 697 as set forth in SEQ ID NO:36 said and said mutant protein depotentiates glutamate receptor activity by altering an allosteric site associated with transmembrane region 5 of said mGluR3.
